(19) United States
(12) Patent Application Publication
(10) Pub. No.: US 2026/0325651 A1
(43) Pub. Date: Oct. 22, 2026

(54) COMPOSITIONS AND METHODS FOR MODULATING MACROPHAGE LIPID METABOLISM AND EFFEROCYTOSIS

(71) Applicant: THE FEINSTEIN INSTITUTES FOR MEDICAL RESEARCH, INC., Manhasset, NY (US)

(72) Inventor: Myoungsun SON, Manhasset, NY (US)

(21) Appl. No.: 19/637,179

(22) Filed: Apr. 2, 2026

Related U.S. Application Data
(60) Provisional application No. 63/791,966, filed on Apr. 21, 2025.
(60) Provisional application No. 63/950,043, filed on Dec. 29, 2025.

(51) Int. Cl. A61K 47/54; A61K 31/444; A61K 31/4706; A61K 31/4709; A61K 45/06; A61K 47/64; A61P 37/06; G01N 33/50

(52) U.S. Cl. CPC A61K 47/542; A61K 31/444; A61K 31/4706; A61K 31/4709; A61K 45/06; A61K 47/643; A61P 37/06; G01N 33/5055

(57) ABSTRACT

The disclosure relates to lipase modulators, compositions comprising such modulators, and methods of using the same to regulate lipid metabolism, efferocytosis, and/or macrophage function. In certain aspects, the disclosure provides compositions and methods for modulating intracellular lipase activity, lipolysis, inflammatory signaling, membrane remodeling, and/or pro-resolving macrophage polarization, including for treating conditions associated with dysregulated lipid metabolism, impaired efferocytosis, and/or impaired macrophage function. The disclosure also provides in vitro methods for identifying candidate agents that increase macrophage efferocytosis, enhance lipase-associated signaling or activity, increase membrane fluidity, increase receptor clustering or receptor polarization, or promote a pro-resolving macrophage phenotype.

### CROSS-REFERENCE TO RELATED APPLICATIONS

This application claims the benefit of and priority under 35 U.S.C. § 119(e) to U.S. Provisional Patent Application No. 63/950,043, filed Dec. 29, 2025 and U.S. Provisional Patent Application No. 63/791,966, filed Apr. 21, 2025, each of which is incorporated herein by reference in their entirety for all purposes.

### STATEMENT OF RIGHTS TO INVENTIONS MADE UNDER FEDERALLY SPONSORED RESEARCH

This invention was made with government support under Grant Nos. AI135063 and AR084084 awarded by the National Institutes of Health (NIH). The government has certain rights in the invention.

### FIELD

The present disclosure is related to regulating lipid metabolism and maintaining lipid homeostasis.

### BACKGROUND

Macrophages have remarkable heterogeneity and play crucial roles as innate myeloid cells in health and disease. The polarization of macrophages into M1 (pro-inflammatory) and M2 (anti-inflammatory or tissue repairing) subtypes is a fundamental aspect of their role in host defense, inflammation, and tissue homeostasis. M1 macrophages, typically induced by cytokines such as IFN-γ and microbial products like lipopolysaccharide (LPS), produce inflammatory cytokines and reactive oxygen species that are essential for antimicrobial defense and tumor suppression. The balance between M1 and M2 macrophages is crucial for maintaining health, and dysregulation of this balance is implicated in various clinical conditions.

Lipid droplets (LDs) are intracellular organelles budding from the endoplasmic reticulum (ER) that function as energy reservoirs and substrates for membrane synthesis. In macrophages and other immune cells, LDs are dynamic structures involved in lipid metabolism, inflammatory responses, and metabolic adaptation. LDs primarily consist of triacylglycerols (TAG), diacylglycerols (DAG), and cholesterol esters (CE), which are hydrolyzed by specific lipases. Adipose triglyceride lipase (ATGL) initiates TAG breakdown, hormone-sensitive lipase (HSL) processes DAGs into monoacylglycerols (MAGs), and monoacylglycerol lipase (MAGL) completes the lipolysis pathway by releasing free fatty acids (FFA).

Efferocytosis, the process of clearing apoptotic cells, is essential for immune homeostasis, inflammation control, and tissue remodeling. This physiological function is vital for maintaining cellular and tissue integrity and preventing autoimmune responses by ensuring the controlled removal of dying cells before they release inflammatory mediators. Dysregulation of efferocytosis induces substantial inflammatory response and is linked with chronic inflammatory diseases, including autoimmune diseases.

However, significant challenges remain in defining macrophage polarization states and the role of LD in efferocytosis. There is a need to elucidate the precise mechanisms by which lipase modulation affect macrophage phenotype, inflammatory responses, and phagocytic capacity. Additionally, there is a need to identify and develop specific lipase modulators and utilizing into effective therapeutic strategies.

### SUMMARY

Various embodiments of the disclosure provide compositions and methods of using such compositions for regulating lipid metabolism, efferocytosis, macrophage function, and combinations thereof. Compositions and articles defined by the embodiments of the disclosure were isolated or otherwise manufactured in connection with the examples provided below. Other features and advantages of the disclosure will be apparent from the detailed description and from the claims.

It is an object to determine how lipid metabolism influences macrophage polarization and efferocytosis. It is another object to develop therapeutic strategies for autoimmune diseases such as Systemic Lupus Erythematosus (SLE) by examining how lipid droplets (LDs), lipases, and fatty acid species influence macrophage polarization and function. It is a further object of the disclosure to identify specific fatty acids enriched in anti-inflammatory macrophages (M2) or pro-resolving macrophages, or that drive monocyte differentiation towards M2, and fatty acids that enhance membrane fluidity, transcriptional programs, mitochondrial function, and resolution pathways. It is one object to determine LD characteristics between inflammatory and anti-inflammatory macrophages and to identify how lipase activity, including hormone-sensitive lipase (HSL) activity, regulates efferocytosis, macrophage function, and lipid metabolism. For example, LD characteristics between lipopolysaccharides (LPS)/interferon-gamma (IFN-γ)-polarized M1 macrophages and interleukin-4 (IL-4)-polarized M2 macrophages, such as the latter exhibiting more numerous and smaller LD. It is an object to inhibit lipase activity (e.g., HSL), in M2 macrophages and shift the macrophages toward an M1-like phenotype, thereby impairing efferocytosis, macrophage function, and lipid metabolism. It is another object to overexpress, activate, or otherwise increase lipase activity to promote a pro-resolving macrophage phenotype, including an M2-like macrophage phenotype, thereby improving efferocytosis, macrophage function, and lipid metabolism.

In some embodiments, the disclosure further provides pharmaceutical compositions that include a therapeutic agent selected from a lipase modulator, nervonic acid or a pharmaceutically acceptable salt thereof, C1q , a phosphodiesterase 3 inhibitor, a beta-adrenergic receptor agonist, or a combination thereof, together with a pharmaceutically acceptable excipient. In some embodiments, the lipase modulator modulates an intracellular lipase, such as adipose triglyceride lipase, hormone-sensitive lipase, monoglyceride lipase, lysosomal acid lipase, N-acylsphingosine amidohydrolase 1, or carboxyl ester lipase. In some embodiments, the therapeutic agent includes nervonic acid, optionally conjugated to serum albumin. In some embodiments, the therapeutic agent includes a phosphodiesterase 3 inhibitor, such as milrinone or cilostazol. In some embodiments, the composition is formulated for parenteral administration, including intraperitoneal administration.

In some embodiments, the disclosure further provides methods for treating dysregulation of lipid metabolism, impaired macrophage efferocytosis, impaired macrophage function, or a disease associated therewith in a subject by administering a therapeutically effective amount of a therapeutic agent selected from a lipase modulator, nervonic acid or a pharmaceutically acceptable salt thereof, C1q , a phosphodiesterase 3 inhibitor, a beta-adrenergic receptor agonist, or a combination thereof, thereby increasing macrophage efferocytosis in the subject. In some embodiments, the disease is an autoimmune disease, including systemic lupus erythematosus or lupus nephritis. In some embodiments, the therapeutic agent includes nervonic acid. In some embodiments, the therapeutic agent includes a phosphodiesterase 3 inhibitor. In some embodiments, administration of the therapeutic agent increases phosphorylation of hormone-sensitive lipase at Ser660 in macrophages of the subject.

In some embodiments, the disclosure further provides in vitro methods for identifying a candidate agent that promotes pro-resolving macrophage activity. Such methods can include contacting a macrophage with a candidate agent, determining whether the macrophage exhibits increased efferocytosis relative to a control macrophage not contacted with the candidate agent, and selecting the candidate agent when the macrophage exhibits increased efferocytosis relative to the control macrophage. In some embodiments, selection of the candidate agent further includes determining increased phosphorylation of hormone-sensitive lipase at Ser660, increased membrane fluidity, or increased receptor clustering or receptor polarization at an apoptotic cell contact site. In some embodiments, the receptor clustering or receptor polarization includes organization of one or more receptors selected from MERTK, TIM-4, CD36, CD206, receptor for advanced glycation end products, leukocyte-associated immunoglobulin-like receptor-1, or a combination thereof. In some embodiments, the macrophage is contacted with the candidate agent in the presence of high mobility group box 1. In some embodiments, the candidate agent is selected from a phosphodiesterase 3 inhibitor, a beta-adrenergic receptor agonist, C1q , nervonic acid, a lipase modulator, or a combination thereof.

### BRIEF DESCRIPTION OF FIGURES

FIGS. 1A-1G show the association of lipid droplet characteristics with enhanced efferocytosis in macrophages. FIG. 1A presents a schematic representation of CD14+ monocyte differentiation into M0 macrophages using macrophage colony-stimulating factor (M-CSF), stained with CytoTell™ Blue, followed by polarization into M1 (IFN-γ/LPS) or M2 (IL-4) macrophages over 24 hours. After polarization, carboxyfulorescein succinimidyl ester (CFSE)-labeled apoptotic Jurkat cells were co-cultured with these macrophages for 2 hours to evaluate the engulfment of apoptotic cells. FIG. 1B exhibits the quantitative assessment of CD36 and CD163 surface expressions in M1 and M2 macrophages, as determined by flow cytometer across all viable cells (n=4 per condition), expressed as geometric mean fluorescence intensity (gMFI). Statistical significance was assessed using a paired t-test. FIG. 1C illustrates a comparison of efferocytosis capacity between M1 and M2 macrophages, indicated by the ratio of CFSE+ macrophages, analyzed with a paired t-test (n=5 per condition). FIG. 1D shows representative cell images and histogram plots illustrating CFSE positivity in M1 and M2 macrophages. BF, Bright field. FIG. 1E presents a quantification of lipid droplet (LD) accumulation in macrophages utilizing HCS LipidTOX™ Green staining; greater LD accumulation was observed in M1 compared to M2 macrophages, analyzed with a paired t-test (n=4 per condition). FIG. 1F exhibits representative images of perilipin-2 (PLIN2) red neutral lipids and 4′,6-diamidino-2-phenylindole (DAPI) blue nuclei (bottom left area) immunostaining in M1 and M2 macrophages. Scale bar=10 μm. FIG. 1G illustrates metrics of LD number and LD volume per cell quantified using Imaris software (n=23 cells per condition, mean±SD), with statistical evaluation conducted using an unpaired t-test. Statistical significance was defined as *P<0.05, **P<0.01, and ***P<0.001. Data represent mean±SEM. All experiments were validated at least three times.

FIGS. 2A-2G shows the impact of adipose triglyceride lipase (ATGL) and hormone-sensitive lipase (HSL) inhibition on efferocytosis in M2-polarized macrophages. FIG. 2A presents differential lipase activity between M1 and M2 macrophages (paired t-test, n=4 per condition). FIG. 2B exhibits the effects of Orlistat, a pan-lipase inhibitor, on efferocytosis in M2-polarized macrophages (far left) that were assessed at concentrations of 50 μM (center) and 100 μM (far right); the proportion of CFSE+ macrophages was measured (one-way ANOVA, n=4 per condition). FIG. 2C illustrates a reduction in efferocytosis in M2 macrophages (far left) following the inhibition of ATGL (Atglistatin, 20 μM; second from left); HSL (HSL-IN-1, 15 μM; second from right); and MAG lipase (MAGL, 5 μM; far right), analyzed via one-way ANOVA (n=4-5 per condition). FIG. 2D shows HSL inhibition (HSL-IN-1, 5 μM; right) reduced efferocytosis in mouse bone marrow-derived M2 macrophages (left) (paired t-test, n=4 per condition). FIGS. 2E-2F presents no significant effect in M2 macrophage (M2 alone, left) efferocytosis with T863 (a DGAT1 inhibitor, DGATi, 50 μM; FIG. 2E, right) or Lalistat1 (a lysosomal lipase inhibitor, LALi, 5 μM; FIG. 2F, right) (paired t-test, n=4 per condition). FIG. 2G exhibits a schematic of lipid droplet (LD) synthesis and degradation pathways in macrophages, depicting key enzymes including glycerol (G), fatty acid (FA), diacylglyceride (DAG), triacylglyceride (TAG), monoacylglycerol (MAG), adipose triglyceride lipase (ATGL), hormone-sensitive lipase (HSL), MAG lipase (MAGL), cholesterol ester (CE), lysosomal lipase (LAL), and lipid droplets (LD). Statistical significance is indicated as *P<0.05, **P<0.01, ***P<0.001, ****P<0.0001, and ns (not significant). Data represent mean±SEM. All experiments were repeated at least three times.

FIGS. 3A-3E illustrates PKA-dependent phosphorylation and LD localization of HSL in M2 Macrophages. FIG. 3A shows high levels of phosphorylation of HSL at Ser660 in M2 macrophages. Immunoblot band intensities were normalized to HSL, and the phosphorylation levels between M1 and M2 differences were compared using densitometry with ImageJ software (n=4 per condition, paired t-test). FIG. 3B presents a visualization of HSL (green) colocalizing with LD, stained with LipidTox™ Deep Red in M1 and M2 macrophages. FIG. 3C exhibits a quantitative assessment of LD-localized HSL. Each dot represents a colocalization voxel per image (n=8 per condition, unpaired t-test). FIG. 3D illustrates PKA activity in M1 and M2 macrophages (n=7 per condition, paired t-test). FIG. 3E shows enhancement of efferocytosis activity in M1 macrophages (M1 alone, left) after treatment with Dibutyryl-cAMP (50 μM; right), a PKA activator, for 24 hours (n=5 per condition, paired t-test). Statistical significance is indicated as *P<0.05, **P<0.01, and ***P<0.001.

FIGS. 4A-4E shows an impact of HSL inhibition on macrophage phenotypic features. FIG. 4A presents transmission electron microscopy displaying lipid droplets (LD) in M1, M2, and M2 macrophages treated with the HSL inhibitor (M2+HSLi); upper row. Enlarged views within the white squares highlight LD (red arrows, bottom row). Magnifications and scale bars are set at 2000× and 2 μm (upper) and 5000× & 0.5 μm (lower). FIGS. 4B-4C exhibits quantitative analysis of LD size (FIG. 4B) and density (FIG. 4C) in M1, M2, and M2+HSLi macrophages performed using ImageJ software. Data represent mean±SD and analyzed via one-way ANOVA. FIG. 4D shows flow cytometer analysis comparing CD163 (left) and CD36 (right) surface expression between M2 and M2+HSLi macrophages (n=6 for CD163, n=4 for CD36 per condition, paired t-test). Data represent mean±SEM. FIG. 4E presents measured levels of TNFα (left) and IL-1β (right) in culture supernatants from M2 and M2+HSLi macrophages (n=5 per condition, paired t-test). Statistical significance was defined as *P <0.05, **P<0.01, ****P<0.0001, and ns (not significant).

FIGS. 5A-5C demonstrate that HSL overexpression enhances efferocytosis and M2 macrophage phenotypes. FIG. 5A shows M0 macrophages transfected with a plasmid encoding HSL (right), Control (left). After 24 hours, efferocytosis was assessed by CFSE positivity among CD14+mCherry+macrophages (n=5 per condition, paired t-test). FIGS. 5B-5C presents measurements of CD163 (FIG. 5B) surface expression and TNFα (FIG. 5C, left) and IL-1β (FIG. 5C, right) levels (n=6 per condition, paired t-test); Control (FIGS. 5B-5C, left). Statistical significance was defined as **P<0.01, ***P<0.001, and ns (not significant). Data represent mean±SEM. All experiments were repeated at least three times.

FIGS. 6A-6B exhibits distinct gene expression profiles between M1 and M2 macrophages and gating strategy for efferocytosis assay. FIG. 6A illustrates gene expression levels of IL-1β, HIF-1α, CD206, MERTK, and PPARγ compared between IFN-γ/LPS (M1) and IL-4 (M2) polarized macrophages using 2\hat{ }−ΔCt (relative expression (R.E.)) analysis. Statistical significance was determined by a paired t-test (*P<0.05 and **P<0.01). FIG. 6B shows the gating strategy for efferocytosis analysis involved gating CytoTell™ Blue-positive macrophages, and CFSE positivity was calculated using single-stained controls.

FIGS. 7A-7D presents the efferocytosis response in lipase-inhibited macrophages. FIG. 7A exhibits histogram plots displaying CFSE positivity in CytoTell™+macrophages treated with Orlistat, a pan-lipase inhibitor. FIG. 7B illustrates histogram plots showing CFSE positivity in CytoTell™+macrophages treated with specific lipase inhibitors: ATGL (Atglistatin, 20 μM), HSL (HSL-IN-1, 5 μM), and MAGL (MJN110, 5 μM). FIG. 7C demonstrates an analysis of efferocytosis activity in M1 macrophages following inhibition of ATGL (Atglistatin, 20 μM), HSL (HSL-IN-1, 15 μM), and MAGL (MJN110, 5 μM), evaluated using one-way ANOVA (n=6 per condition). FIG. 7D shows stable efferocytosis response in M1 macrophages after treatment with a DGAT inhibitor (n=4, analyzed using a paired t-test). Statistical significance was defined as ***P<0.001, and ns (not significant). Data represent mean±SEM. Experiments were repeated at least twice.

FIGS. 8A-8G illustrate restoration of pro-resolving macrophage function by nervonic acid (NA) (FA 24:1) in vitro and in vivo. FIG. 8A shows that exogenous FA 24:1 treatment of inflammatory M1 macrophages increases efferocytosis in human and mouse M1 macrophages of in vitro mouse models. FIG. 8B shows that FA 24:1 treatment increases peroxisome proliferator-activated receptor gamma (PPARγ) expression in M1 macrophages demonstrating that this functional rescue is associated with an increase in PPARγ expression. FIG. 8C shows that FA 24:1 treatment increases mitochondrial respiration (mitochondrial oxygen consumption rate (OCR)) in M1 macrophages, a key metabolic feature of pro-resolving cells, as determined by Seahorse analysis. FIG. 8D shows that peritoneal macrophages from SLETC mice (i.e., model of impaired efferocytosis) exhibit increased lipid accumulation (LipidTox staining) relative to wild-type (WT) controls. FIG. 8E shows (left panel) provides a schematic of the in vivo sterile peritonitis model used to assess efferocytosis. FIG. 8E (right panel) presents representative flow plots showing gating for phagocytosed (CFSE+) apoptotic cells. FIG. 8F shows reduced efferocytosis in peritoneal myeloid cells from HMGB1-treated wild-type mice and MerTK-deficient mice (i.e., MerTK knockout (KO) mice serve as a negative control for efferocytosis). FIG. 8G shows that systemic administration of FA 24:1 restores efferocytosis in HMGB1-treated wild-type mice and in SLETC mice. Data are shown as box-and-whisker plots or mean±SEM from at least three independent experiments. Statistical significance was determined by an unpaired Student's t-test (FIG. 8B, FIG. 8D) or a one-way ANOVA with Tukey's multiple comparisons test (FIG. 8A, FIG. 8F, FIG. 8G). *p<0.05, **p<0.01, ***p<0.001.

FIGS. 9A-9D shows that C1q and HMGB1 restore efferocytosis function through a PKA-HSL-dependent lipolytic pathway. Human monocyte-derived macrophages were polarized to an M1-like state with HMGB1 or an M2-like state with HMGB1+C1q. FIG. 9A provides representative immunofluorescence images show staining for the lipid droplet marker PLIN2 (red) and nuclei (DAPI, blue). M1-like macrophages display large LDs, while M2-like macrophages show smaller LDs. Scale bar=10 μm. FIG. 9B illustrates mitochondrial function assessed by Seahorse XF analysis. M2-like macrophages exhibit significantly higher basal and maximal Oxygen Consumption Rate (OCR) compared to M1-like, untreated, or C1q-treated cells, indicating restored metabolic fitness. The inset shows a higher OCR/ECAR ratio in M2-like cells, confirming a shift toward oxidative metabolism. FIG. 9C demonstrates that M2-like macrophages show increased PKA activity compared to M1-like cells. FIG. 9D shows the efferocytosis capacity as measured by flow cytometry. M2-like macrophages show robust efferocytosis, which is abrogated by treatment with a specific HSL inhibitor (HSLi). Each dot represents an individual donor. *P<0.05, **P<0.01, ****P<0.0001.

FIGS. 10A-10F demonstrate that dispersed lipid droplets (LDs) and lipolysis of LD components affect the efferocytosis of macrophages. FIG. 10A presents a schematic representation of the differentiation process of M1, M1-like, M2, and M2-like macrophages from monocytes of healthy controls (HD) or SLE patients. FIG. 10B provides a visualization of LD characteristics stained with anti-perilipin 2 (PLIN2) antibody in M1/M2 macrophages (left) and M1-like/M2-like macrophages (right). Images obtained from Aryscan of z-stacks with a 0.13 μm interval between slices (LSM 900 confocal images, 63× oil). FIG. 10C illustrates the total LD volume per cell quantified using Imaris software. FIG. 10D shows transmission electron microscopy (TEM) images displaying LDs (red arrows) in M1 and M2 macrophages, captured by the Electron Microscopy Analytical Imaging Facility at Albert Einstein College of Medicine. Analysis of LD area (μm\hat{ }2) and density conducted using ImageJ (left panel). Brightness (LD density) is represented by values from 255 (white) to 0 (black) (right panel). FIG. 10E demonstrates percent efferocytosis as measured by an efferocytosis assay involving M1 and M2 macrophages in the presence of specific lipase inhibitors. Staurosporin-treated Jurkat cells (apoptotic cells) co-cultured with macrophages for 2 hours at a 2:1 ratio. Comparisons between M1 and M2, M1 with Hsl inhibitor (+HSLi) versus without, and M2 with indicated lipase inhibitors were highlighted in the analysis. FIG. 10F illustrates percent efferocytosis as measured by efferocytosis assays performed from isolated CD14+CD16−monocytes from HD or SLE patients. HMGB1 or HMGB1 plus C1q were treated to generate M1-like or M2-like macrophages. Results are presented as medians, with individual donor data points shown with at least duplicates. Statistical analysis was conducted using One-Way ANOVA, with significance levels denoted as follows: *p<0.05; **p<0.01; ***p<0.001; ****p<0.0001.

FIGS. 11A-11H demonstrate that PKA-mediated HSL activation drives efferocytosis in macrophages. FIG. 11A shows that efferocytosis efficiency is highest in M2 macrophages. FIG. 11B illustrates that M1 macrophages accumulate larger and more numerous LDs per cell than M2 macrophages. FIG. 11C presents elevated lipase activity in M2 macrophages. FIG. 11D demonstrates HSL inhibition that impairs efferocytosis in M2 macrophages. FIG. 11E shows HSL inhibition that reduces efferocytosis in murine BMDMs. FIG. 11F illustrates that HSL inhibition in M2 macrophages increases LD volume, mimicking the M1 profile. FIG. 11G presents PKA activity that is higher in M2 macrophages than M1 macrophages. FIG. 11H presents immunoblotting confirmation that HSL phosphorylation (at Serine 660; pHSL S660: PKA activation site) is elevated in M2 macrophages; HSL inhibition reduces it. Each dot shows an independent donor except in B. *P<0.05, **P<0.01, ***P<0.001, ****P<0.0001; ns, not significant.

FIGS. 12A-12C illustrates that HSL promotes M2 macrophage polarization. FIG. 12A shows that siRNA-mediated knockdown of HSL (LIPE) in human macrophages reduces efferocytosis and Annexin A1 secretion and increases TNFα production. FIG. 12B demonstrates that HSL overexpression in M0 macrophages increases efferocytosis and Annexin A1 secretion and reduces inflammatory cytokines (TNFα, IL-1β), accompanied by increased expression of M2-associated genes (PPARγ, CD36, CD200R1). FIG. 12C demonstrates that HSL overexpression increases expression of fatty acid oxidation (FAO) genes (CPT1A, ACADVL) and enhances mitochondrial respiration measured by Seahorse XF₉₆ Mito Stress Test (Maximal OCR, spare respiratory capacity). *P<0.05, **P<0.01, ***P<0.001.

FIGS. 13A-13D show that C1q and HMGB1 restore efferocytosis in an HSL-dependent manner. FIG. 13A demonstrates that HMGB1 alone accumulates LDs, while HMGB1+C1q co-treatment reduces LD size. FIG. 13B presents Seahorse analysis that C1q- and HMGB1-exposed M2-like cells have smaller LDs and restored OCR and OCR/ECAR ratio, similar to IL-4-induced M2 cells. FIG. 13C illustrates that PKA activity is significantly high in M2-like macrophages. FIG. 13D shows that HSL inhibition reduced efferocytosis from M2-like macrophages. *P<0.05, **P<0.01, ****P<0.0001.

FIG. 14 illustrates an integrated miRNA-mRNA network analysis. The analysis found reduced PDE3A expression in HMGB1+C1q macrophages and increased miRNAs targeting PDE3A. Nodes represent miRNAs (red: miR-3614-5p; miR-449c-5p; miR-6824-3p; miR-548av-5p) and PDE3A (blue: CATSPER2; PDE3A; NUGGC) with edges for inhibitory interactions.

FIGS. 15A-15C show that HMGB1 and C1q co-injection promote M2-like macrophages in sterile peritonitis. FIG. 15A presents an ELISA showing decreased leukotriene B4 in peritoneal exudates from C1q+HMGB1-treated mice and resolving D (FIG. 15B). FIG. 15C shows RT-PCR confirmation of M1/M2 gene signatures depending on treatment.

FIGS. 16A-16B illustrate plasma membrane fluidity and surface expression of scavenger receptors (SRs). FIG. 16A shows membrane fluidity of M0, M1, and M2 macrophages, with or without HSL inhibitor (HSLi, 5 μM, 24 h) (left) and of M1-like, M2-like, and C1q alone(24 h, right). FIG. 16B demonstrates flow cytometry of CD206 and CD36 on macrophages. The HSL inhibitor was applied for 3 hours (5 μM). One-way ANOVA. *P<0.05, *P<0.01.

FIGS. 17A-17B demonstrate enrichment of nervonic acid (FA 24:1) in M2 macrophages compared to M1 cells. FIG. 17A illustrates absolute quantification of FA 24:1 and ceramide (d18:1/24:1) in M1 and M2 macrophages, expressed as nmol per 5×10⁵ cells. (n=6). FIG. 17B shows acid ceramidase (ASAH1) expression in M1 and M2 macrophages. Left, ELISA quantification of secreted ASAH1 in culture supernatants (n=5). Right, relative ASAH1 mRNA expression (n =3). *P<0.05, *P<0.01.

### DETAILED DESCRIPTION

The embodiments of the disclosure feature compositions and methods of use, and the disclosure is based, at least in part, on the discovery that modulation of enzymes that hydrolyze triglycerides (TGs) and/or their activities can impact macrophage function, and their efferocytosis capacity (i.e., phagocytic clearance of apoptotic cells). Also, the discovery that macrophage efferocytosis and pro-resolving function are regulated by intracellular lipase-mediated lipid droplet remodeling, particularly through pathways involving hormone-sensitive lipase and related metabolic signaling. The disclosure is further based on the discovery that enhancing these pathways, including through HMGB1/C1q-regulated programming, PDE3A-cAMP-PKA signaling, and nervonic acid-associated lipid remodeling, can restore macrophage metabolic fitness and improve clearance of apoptotic cells . Additionally, it was an object of the disclosure to determine how lipid metabolism influences macrophage polarization and efferocytosis. Distinct lipid droplet (LD) characteristics between LPS/IFN-γ-polarized M1 macrophages and IL-4-polarized M2 macrophages were found, with the latter exhibiting more numerous and smaller LDs. Inhibition of lipase activity, such as hormone-sensitive lipase (HSL), in M2 macrophages was found to alter LD characteristics and transfers the macrophages toward an M1-like phenotype and notably impairing efferocytosis. Conversely, HSL overexpression restores these functions and promotes an M2-like macrophage phenotype.

In particular, inhibition of exemplary enzymes, adipose triglyceride lipase (ATGL) and hormone-sensitive lipase (HSL), using a pan-lipase inhibitor (e.g., Orlistat), ATGL inhibitor, and HSL inhibitor, reduced the efferocytosis capacity of M2 macrophages derived from human peripheral monocytes. Conversely, overexpression of ATGL and HSL in macrophages significantly increased their efferocytosis activity. Similar results were observed in murine bone marrow-derived macrophages. Modulation of ATGL and HSL activities influences macrophage function, including their ability to maintain tissue homeostasis through the clearance of apoptotic cells. The impairment of efferocytosis by inhibition of enzymes that play a role in the breakdown of triglycerides (such as but not limited to ATGL and HSL) contributes to the development and progression of diseases that require efficient clearance of apoptotic cells for the resolution of inflammation and the reduction or prevention of autoimmune responses.

In some embodiments, defective efferocytosis is associated with inflammatory signals that promote lipid droplet accumulation and impair macrophage metabolic fitness. In some embodiments, high mobility group box 1 (HMGB1) promotes an inflammatory macrophage state associated with increased lipid droplet accumulation, reduced mitochondrial fitness, and impaired efferocytosis. In some embodiments, complement component C1q opposes HMGB1-associated dysfunction and promotes a pro-resolving macrophage state. In some embodiments, HMGB1, C1q , or a downstream pathway regulated thereby is a therapeutic target for treatment of systemic lupus erythematosus, lupus nephritis, or a related inflammatory or autoimmune condition. In some embodiments, extracellular HMGB1 is associated with impaired efferocytosis by a macrophage through interference with recognition or clearance of an apoptotic cell. In some embodiments, C1q counteracts one or more HMGB1-associated effects and promotes a pro-resolving macrophage state characterized by increased efferocytosis, increased oxidative metabolism, increased protein kinase A activity, increased hormone-sensitive lipase activity, decreased lipid droplet burden, altered lipid droplet composition, or a combination thereof. In some embodiments, treatment includes increasing C1q activity, decreasing HMGB1 activity, disrupting HMGB1 signaling, increasing responsiveness to C1q , or combinations thereof. In some embodiments, such modulation restores macrophage homeostasis in an inflammatory microenvironment associated with SLE. In some embodiments, macrophages exposed to HMGB1 and C1q exhibit reduced lipid droplet size, increased oxidative metabolism, increased protein kinase A (PKA) activity, increased lipase-dependent lipid remodeling, and increased efferocytosis relative to macrophages exposed to HMGB1 alone. In some embodiments, the disclosed pathways are relevant to autoimmune disease, including systemic lupus erythematosus (SLE), in which impaired macrophage clearance of apoptotic cells contributes to chronic inflammation and loss of immune homeostasis.

In some embodiments, complement component C1q and high mobility group box 1 protein regulate macrophage activation state, metabolic programming, and efferocytosis capacity. In some embodiments, C1q promotes a reparative macrophage phenotype, whereas high mobility group box 1 protein promotes inflammatory macrophage activation. In some embodiments, combined signaling involving C1q and high mobility group box 1 protein modulates expression of efferocytosis-associated receptors, lipid remodeling pathways, mitochondrial metabolism, and pro-resolving activity. In some embodiments, macrophages exposed to C1q under conditions of inflammatory challenge exhibit increased MerTK expression, increased lipid droplet lipolysis, increased mitochondrial respiration, and increased efferocytosis.

Lipid droplet remodeling can act as a mechanistic control point in macrophage immunometabolism. In some embodiments, hormone-sensitive lipase mediates hydrolysis of neutral lipids stored in lipid droplets, thereby mobilizing lipid substrates that support membrane remodeling, mitochondrial metabolism, and efferocytic function. In some embodiments, hormone-sensitive lipase activity is regulated by cyclic AMP-dependent protein kinase signaling, including phosphorylation at activating residues and recruitment of hormone-sensitive lipase to lipid droplets. In some embodiments, activation of the protein kinase A-hormone-sensitive lipase pathway increases lipid droplet lipolysis, enhances oxidative metabolism, promotes membrane remodeling, and improves efferocytosis in macrophages.

In some embodiments, HMGB1 and C1q engage a cyclic adenosine 3′,5′-monophosphate-protein kinase A-hormone-sensitive lipase pathway in a macrophage, thereby increasing hormone-sensitive lipase activation, promoting lipid droplet lipolysis, and converting the macrophage to an efferocytosis-competent state. In some embodiments, lipid droplet hydrolysis releases one or more fatty acids or related lipid species that are used to support mitochondrial oxidative metabolism, membrane remodeling, efferocytosis, or a combination thereof. In some embodiments, macrophages exposed to HMGB1 in combination with C1q exhibit increased cyclic adenosine 3′,5′-monophosphate-protein associated signaling, increased protein kinase A activity, increased phosphorylation of hormone-sensitive lipase at Ser660, reduced lipid droplet burden, increased mitochondrial respiration, and increased efferocytosis relative to macrophages exposed to HMGB1 alone. In some embodiments, defective activation of the cyclic adenosine 3′,5′-monophosphate-protein kinase A-hormone-sensitive lipase pathway contributes to impaired efferocytosis in a macrophage from a subject having systemic lupus erythematosus, and restoration of such pathway is used to improve pro-resolving macrophage activity.

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the composition, unless otherwise defined.

As used herein, the term “a” or “an” means one or more, and, when used in conjunction with the term “including,” means one or more than one. As used herein, the term “another” means at least a second or more. As used herein, the term “increase,” “increases,” or “increasing” refers to any detectable elevation relative to a suitable reference, including an elevation that is statistically significant or biologically meaningful. As used herein, the term “range” or “ranges” means that all numeric ranges disclosed herein include the recited endpoints and all intermediate values, including half-integer values where applicable, and further include all subranges subsumed within the disclosed range. As used herein, the term “reduce,” “reduces,” or “reducing” refers to any detectable decrease relative to a suitable reference, including a decrease that is statistically significant or biologically meaningful. As used herein, the term “weight %” refers to weight percent of a component relative to the total weight of the relevant composition, and the sum of all weight % values of individual components in a given composition does not exceed 100%.

### Lipase Modulators

Macrophage lipid metabolism refers to various processes by which macrophages treat lipids, including but not limited to their uptake, storage, degradation, use in various cellular functions. These processes play a role in macrophage activation, polarization, immune responses, and disease progression. Targeting macrophage polarization to modulate an immune response is a therapeutic strategy for treating, for example, autoimmune diseases, cancer, chronic inflammation, and obesity. As used herein, the term “macrophage” refers to a myeloid cell capable of phagocytic activity, including a monocyte-derived macrophage, a tissue macrophage, or a macrophage derived from a subject having an inflammatory, autoimmune, neurodegenerative, or other disease disclosed herein. As used herein, the term “macrophage function” refers to one or more measurable macrophage activities, including efferocytosis, polarization-associated marker expression, cytokine production, lipid droplet remodeling, oxidative metabolism, mitochondrial respiration, membrane remodeling, or serial engulfment capacity. As used herein, the term “dysregulation of lipid metabolism” refers to an abnormality in lipid uptake, storage, lipolysis, trafficking, utilization, or remodeling in a cell, including a macrophage.

In some embodiments of the disclosure, the failure to efficiently clear apoptotic cells (i.e., efferocytosis) in, for example, systemic lupus erythematous (SLE) contributes to chronic inflammation. By improving lipase activity, such as of those lipases involved in lipid metabolism and energy regulation within cells, including intracellular lipases, efferocytosis is enhanced and inflammation resolution is promoted. In some embodiments of the disclosure, lipase modulators, such as but not limited to lipase activators, increase lipase activity thereby enhances efferocytosis and reduces inflammation. Non-limiting examples of lipases of lipase activators include: adipose triglyceride lipase (ATGL), hormone-sensitive lipase (HSL), monoglyceride lipase (MGL), lysosomal acid lipase (LAL), ceramidase (CDase; e.g., N-acylsphingosine amidohydrolase 1 (ASAH1)), and carboxyl ester lipase (CEL). As used herein, the term “monoglyceride lipase” or “MGL” or “MAGL” refers to a lipase that hydrolyzes monoacylglycerol substrates. As used herein, the term “lysosomal acid lipase” or “LAL” refers to a lysosome-associated lipase involved in intracellular lipid processing. As used herein, the term “carboxyl ester lipase” or “CEL” refers to a lipase capable of hydrolyzing esterified lipid substrates. Such lipase activators are useful in improving efferocytosis in the treatment of diseases associated with impaired efferocytosis.

As used herein, the term “efferocytosis” refers to the phagocytic clearance of apoptotic cells by a macrophage or other phagocytic cell. As used herein, the term “impaired macrophage efferocytosis” refers to a reduced capacity of a macrophage to engulf, internalize, process, or clear apoptotic cells relative to a reference macrophage, reference condition, or healthy state. As used herein, the term “intracellular lipase” refers to a lipase that functions within a cell to hydrolyze one or more lipid substrates associated with lipid metabolism, energy regulation, lipid droplet turnover, membrane remodeling, or a combination thereof. As used herein, the term “lipase modulator” refers to an agent that increases or decreases the activity, expression, localization, or function of one or more lipases involved in intracellular lipid metabolism, lipid droplet remodeling, or both. As used herein, the term “lipase activator” refers to a lipase modulator that increases the activity, expression, localization, or function of a lipase associated with lipid droplet lipolysis, macrophage lipid remodeling, efferocytosis, or a combination thereof.

In some embodiments, inhibiting lipases (for example, ATGL, HSL, MGL, LAL, CDase, CEL) and/or using lipase inhibitors, can disrupt lipid metabolism, thereby enhancing anti-tumor immunity and reducing tumor-producing effects. In certain diseases associated with cancers, such as those affected by dysregulation of lipid metabolism, tumor-associated macrophages (TAMs) create an environment that supports tumor growth and suppresses an anti-tumor immune response.

As used herein, the term “lipase inhibitor” refers to a lipase modulator that decreases the activity, expression, localization, or function of a lipase involved in lipid metabolism, lipid droplet turnover, or both. Inhibition of lipases, such as ATGL and HSL, using Orlistat (a pan-lipase inhibitor), ATGL inhibitor, and HSL inhibitor, reduces the efferocytosis capacity of M2 macrophages derived from human peripheral monocytes. Conversely, overexpression of ATGL and HSL in macrophages significantly increased their efferocytosis activity. Similar results were observed in murine bone marrow-derived macrophages.

These findings suggest that lipase modulators such as those of ATGL and HSL activity can influence macrophage function, particularly their ability to maintain tissue homeostasis through the clearance of apoptotic cells. The impairment of efferocytosis by ATGL and HSL inhibition contributes to the development and progression of diseases like SLE, where the efficient clearance of apoptotic cells is important for the resolution of inflammation and the prevention of autoimmune responses.

In some embodiments, lipase modulators are useful as therapeutic agents for treating dysregulation of lipid metabolism, including but not limited to SLE and related metabolic disorders. For example, ATGL and HSL modulators enhance macrophage function. Accordingly targeted activation of lipases (e.g., ATGL, HSL, etc.) in macrophages, improves phagocytic capacity and the ability to engage in efferocytosis, which is important for maintaining tissue homeostasis and preventing or reducing the development of diseases such as SLE.

In some embodiments, ATGL and HSL activators or compositions including such lipase activators, enhance macrophage function, improve lipid metabolism, and improve or increase efferocytosis. Some objects of the disclosure provide ATGL and HSL activators that treat SLE and related metabolic disorders, such as but not limited to atherosclerosis and non-alcoholic fatty liver disease. Additional objects of the disclosure are directed to the activation of PKA or use of PKA activators for increasing (i.e., enhancing, improving) efferocytosis, promoting LD lipolysis, increasing (i.e., enhancing, improving) lipase activity (e.g., HSL via phosphorylation at Ser660), or combinations thereof.

### Pharmaceutical Compositions and Therapeutic Administration

In some embodiments, a composition or pharmaceutical composition, used interchangeably here, including a lipase modulator is provided for use in the manufacture of a medicament for the treatment of a disease or condition associated with dysregulation of lipid metabolism, impaired efferocytosis, impaired macrophage function, or a combination thereof. In some embodiments, the medicament is manufactured for administration to a subject having an autoimmune disease, an inflammatory disease, a neurodegenerative disease, diabetes, cancer, chronic obstructive pulmonary disease, atherosclerosis, multiple sclerosis, lupus nephritis, systemic lupus erythematosus, or a combination thereof. In some embodiments, the lipase modulator includes a lipase activator that increases efferocytosis, increases macrophage lipid metabolism, promotes a pro-resolving macrophage phenotype, or a combination thereof. In some embodiments, the lipase modulator includes a lipase inhibitor useful in a disease setting in which inhibition of lipid metabolism in a macrophage is beneficial, including a tumor-associated macrophage setting. In some embodiments, manufacture of the medicament includes formulating the lipase modulator with one or more pharmaceutically acceptable excipients suitable for the intended route of administration.

As used herein, the term “composition” refers to a formulation including at least one active agent disclosed herein, optionally together with one or more pharmaceutically acceptable excipients, for administration to a subject or for contacting a cell. As used herein, the term “subject” refers to a mammal in need of treatment, including a human, dog, cat, horse, cow, goat, and the like.

In some embodiments, a composition or pharmaceutical composition, used interchangeably here, of the disclosure includes a lipase modulator and one or more pharmaceutically acceptable excipients. In some embodiments, the pharmaceutically acceptable excipient includes a carrier, diluent, binder, filler, disintegrant, lubricant, preservative, stabilizer, buffering agent, tonicity agent, solubilizing agent, suspending agent, surfactant, emulsifying agent, or a combination thereof. In some embodiments, the composition is formulated as a solution, suspension, syrup, dispersion, emulsion, liposomal composition, nanoparticle formulation, tablet, capsule, chewable, powder, granule, injectable formulation, depot formulation, lyophilized preparation for reconstitution, or the like. In some embodiments, the composition is formulated for oral, parenteral, intravenous, intraperitoneal, subcutaneous, intramuscular, intradermal, inhalational, intranasal, topical, or local administration. In some embodiments, the pharmaceutical composition is configured to preserve activity of the lipase modulator, improve bioavailability, improve stability, reduce toxicity, facilitate delivery to monocytes or macrophages, or a combination thereof.

As used herein, the term “pharmaceutically acceptable excipient” refers to a non-active ingredient suitable for use in a pharmaceutical formulation, including, for example, a binder, carrier, diluent, disintegrant, filler, lubricant, preservative, stabilizer, or combination thereof. As used herein, the phrase “consist essentially of” or “consisting essentially of” means that the relevant composition, formulation, or ingredients described here include the listed components together with normal impurities present in commercial materials and optional additives present at levels that do not materially affect the operation of the disclosed embodiments.

### Methods of Treatment and Therapeutic Uses

In some embodiments, a method of treating dysregulation of lipid metabolism, impaired efferocytosis, impaired macrophage function, or a disease associated therewith in a subject, includes: administering to the subject a therapeutically effective amount of an agent or a therapeutic agent selected from the group consisting of a lipase modulator, nervonic acid or a pharmaceutically acceptable salt thereof, C1q , a phosphodiesterase 3 inhibitor, a beta-adrenergic receptor agonist, and combinations thereof, thereby treating dysregulation of lipid metabolism, impaired efferocytosis, impaired macrophage function, or the disease associated therewith in a subject. In some embodiments, a composition including a lipase modulator is provided for the treatment of a subject in need thereof or use in the manufacture of a medicament for the treatment of a disease or condition associated with dysregulation of lipid metabolism, impaired efferocytosis, impaired macrophage function, or a combination thereof. In some embodiments of the methods disclosed here, the medicament is manufactured for administration to a subject having an autoimmune disease, an inflammatory disease, a neurodegenerative disease, diabetes, cancer, chronic obstructive pulmonary disease, atherosclerosis, multiple sclerosis, lupus nephritis, systemic lupus erythematosus, or a combination thereof. In some embodiments, the lipase modulator includes a lipase activator that increases efferocytosis, increases macrophage lipid metabolism, promotes a pro-resolving macrophage phenotype, or a combination thereof. In some embodiments, the lipase modulator includes a lipase inhibitor useful in a disease setting in which inhibition of lipid metabolism in a macrophage is beneficial, including a tumor-associated macrophage setting. In some embodiments, manufacture of the medicament includes formulating the lipase modulator with one or more pharmaceutically acceptable excipients suitable for the intended route of administration.

As used herein, the term “therapeutically effective amount” refers to an amount of an agent or composition sufficient to produce a desired biological effect in a subject, including increasing macrophage efferocytosis, improving macrophage lipid metabolism, promoting a pro-resolving macrophage phenotype, or treating a disease associated with dysregulation of lipid metabolism, efferocytosis, or both. As used herein, the term “treat,” “treating,” or “treatment” refers to preventing, reducing, inhibiting, delaying onset of, slowing progression of, ameliorating, or reversing one or more signs, symptoms, biomarkers, cellular defects, tissue defects, or clinical manifestations of a disease or disorder. The term “consists essentially of” or “consisting essentially of,” as used herein, limits the scope of the ingredients and steps to the specified materials or steps and those that do not materially affect the basic and novel characteristic(s) of the present disclosure, i.e., pharmaceutical compositions. methods for treating dysregulation of lipid metabolism, impaired macrophage efferocytosis, impaired macrophage function, or a disease associated therewith in a subject; methods of identifying a candidate agent that promotes pro-resolving macrophage activity.

In some embodiments, a therapeutically effective amount of an agent disclosed herein is an amount effective to increase macrophage efferocytosis, improve macrophage lipid metabolism, promote a pro-resolving macrophage phenotype, improve mitochondrial respiration, or treat a disease associated with dysregulation of lipid metabolism, impaired macrophage efferocytosis, or impaired macrophage function. In some embodiments in which the agent includes nervonic acid, the therapeutically effective amount includes about 1 mg/kg or greater (e.g., 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75); 100 mg/kg or less (e.g., 98, 96, 94, 92, 90, 88, 86, 84, 82, 80, 78, 76, 74, 72, 70, 68, 66, 64, 62, 60, 58, 56, 54, 52, 50, 48, 46, 44, 42, 40, 38, 36, 34, 32, 30, 28, 26, 24, 22, 20, 18, 16, 14, 12, 10, 8, 6, 4, 2, 0.8); or 1 mg/kg-100 mg/kg (e.g., 5 mg/kg-95 mg/kg; 10 mg/kg-90 mg/kg; 15 mg/kg-85 mg/kg; 20 mg/kg-80 mg/kg; 25 mg/kg-75 mg/kg; 30 mg/kg-70 mg/kg; 35 mg/kg-65 mg/kg; 40 mg/kg-60 mg/kg; 45 mg/kg-55 mg/kg), optionally by intraperitoneal administration. In some embodiments, a single administration of nervonic acid at about 10 mg/kg is sufficient to restore or increase efferocytosis in vivo. In some embodiments, repeated administration of nervonic acid includes about 10 mg/kg intraperitoneally on alternating days for about 2 weeks to about 6 weeks, optionally beginning at about 20 weeks of age. In some embodiments, nervonic acid is formulated as a free fatty acid, as a pharmaceutically acceptable salt, as a complex with a carrier protein, or as part of a delivery system suitable for increasing availability to a macrophage or monocyte. In some embodiments, where the disclosed agent is evaluated in vitro or ex vivo, macrophages are contacted with nervonic acid at about 1 μM to about 50 μM, including about 10 μM, optionally for about 24 hours. In some embodiments, an experimental concentration useful for evaluating modulation of macrophage efferocytosis or lipid metabolism includes Orlistat at about 10 μM to about 100 μM, including about 50 μM or about 100 μM, Atglistatin at about 20 μM, HSL-IN-1 at about 5 μM to about 15 μM, MJN110 at about 5 μM, T-863 at about 50 μM, Lalistat 1 at about 5 μM, or dibutyryl-cAMP at about 50 μM. In some embodiments, such in vitro or ex vivo concentrations are useful for screening, mechanistic evaluation, or functional assessment of macrophage efferocytosis, lipid droplet remodeling, membrane fluidity, receptor organization, inflammatory signaling, or combinations thereof, and may inform selection of an agent for therapeutic administration to a subject. The methods described throughout the disclosure employ various routes of administration, including but not limited to intravenous, subcutaneous, oral, intramuscular, intradermal, inhalational, intranasal, topical, local, or parenteral administration, including intraperitoneal administration, and compositions formulated for such routes of administration.

In some embodiments, the amount of an agent administered to a subject, the frequency of administration (e.g., once, twice, three, or more times per day; every other day; every two days; as needed), the duration of treatment (e.g., one day, two days, three days, or more days; one week, two weeks, three weeks, four weeks, five weeks, six weeks, or more; one month, two months, three months, four months, five months, six months, or more; one year, two years, three years, four years; until the subject no longer has dysregulation of lipid metabolism, impaired macrophage efferocytosis, impaired macrophage function, or the disease or condition associated therewith in the subject), and the dosage regimen are selected by a medical practitioner based on factors including the subject's age, sex, weight, body surface area, general health, nutritional status, renal function, hepatic function, disease type, disease stage, disease severity, inflammatory status, immune status, comorbidities, concomitant medications, route of administration, formulation, and observed clinical response. In some embodiments, the medical practitioner may initiate treatment at a first amount and adjust the amount, dosing interval, or duration based on tolerability, pharmacodynamic response, biomarker response, or therapeutic outcome. In some embodiments, the therapeutically effective amount may vary among subjects and may be determined according to routine clinical practice using no more than ordinary skill in the art. In some embodiments, the dosage regimen is selected to increase macrophage efferocytosis, improve macrophage lipid metabolism, promote a pro-resolving macrophage phenotype, reduce inflammation, or treat a disease or condition disclosed herein while reducing unwanted effects. In some embodiments, treatment is administered as a single dose, multiple doses, a repeated intermittent regimen, or a continuous regimen, and may be adjusted during the course of treatment based on clinical evaluation of the subject.

### HMGB1/C1q-Regulated Macrophage Programming

In some embodiments, activation of the PKA-HSL pathway is achieved by modulating upstream inflammatory and pro-resolving signaling pathways. In some embodiments, HMGB1 promotes an inflammatory macrophage state associated with lipid droplet accumulation and impaired efferocytosis, whereas C1q , including C1q in combination with HMGB1, promotes a macrophage phenotype having increased PKA activity, increased HSL activation, increased lipid droplet lipolysis, increased mitochondrial respiration, and increased efferocytosis. In some embodiments, the HMGB1/C1q-regulated pathway is mediated, at least in part, by receptor-associated signaling including receptor for advanced glycation end products (RAGE), leukocyte-associated immunoglobulin-like receptor-1 (LAIR-1), or both.

As used herein, the term “HMGB1” refers to high mobility group box 1. As used herein, the term “C1q ” refers to complement component C1q . As used herein, the term “inflammatory macrophage state” refers to a macrophage state associated with reduced efferocytosis, increased inflammatory mediator production, increased lipid droplet accumulation, reduced metabolic fitness, or a combination thereof. As used herein, the term “pro-resolving macrophage” or “pro-resolving macrophage phenotype” refers to a macrophage state associated with enhanced efferocytosis, increased lipid remodeling, increased oxidative metabolism, increased mitochondrial fitness, increased membrane fluidity, increased expression or activity of one or more resolution-associated markers, or a combination thereof. As used herein, the term “mitochondrial respiration” refers to oxidative mitochondrial activity, including one or more of basal respiration, maximal respiration, ATP production, spare respiratory capacity, oxygen consumption, or related measures of mitochondrial function.

In some embodiments, HMGB1 signaling through receptor for advanced glycation end products and C1q signaling through leukocyte-associated immunoglobulin-like receptor-1 cooperate to regulate macrophage lipid remodeling and efferocytosis. In some embodiments, such receptor-associated signaling increases cyclic adenosine 3′,5′-monophosphate signaling, increases protein kinase A activity, increases hormone-sensitive lipase activation, increases adipose triglyceride lipase activity, or a combination thereof. In some embodiments, adipose triglyceride lipase is activated directly or indirectly through protein kinase A, AMP-activated protein kinase, or both. In some embodiments, HMGB1 in combination with C1q increases AMP-activated protein kinase phosphorylation in a macrophage. In some embodiments, lipolysis supporting a pro-resolving macrophage phenotype includes coordinated activity of hormone-sensitive lipase and adipose triglyceride lipase. In some embodiments, modulation of phosphodiesterase 3A, including by a phosphodiesterase 3 inhibitor or by a microRNA that decreases phosphodiesterase 3A expression, is used to increase cyclic adenosine 3′,5′-monophosphate and support the receptor-associated signaling pathway.

As used herein, the term “cAMP-protein kinase A-hormone-sensitive lipase pathway,” “cyclic adenosine monophosphate-protein kinase A-hormone-sensitive lipase pathway,” or “cAMP-PKA-HSL pathway” refers to a signaling pathway in which increased intracellular cyclic adenosine monophosphate promotes protein kinase A activity and thereby increases activation of hormone-sensitive lipase. As used herein, the term “protein kinase A” or “PKA” refers to a kinase activated by cyclic adenosine monophosphate and associated with phosphorylation and activation of hormone-sensitive lipase in disclosed embodiments.

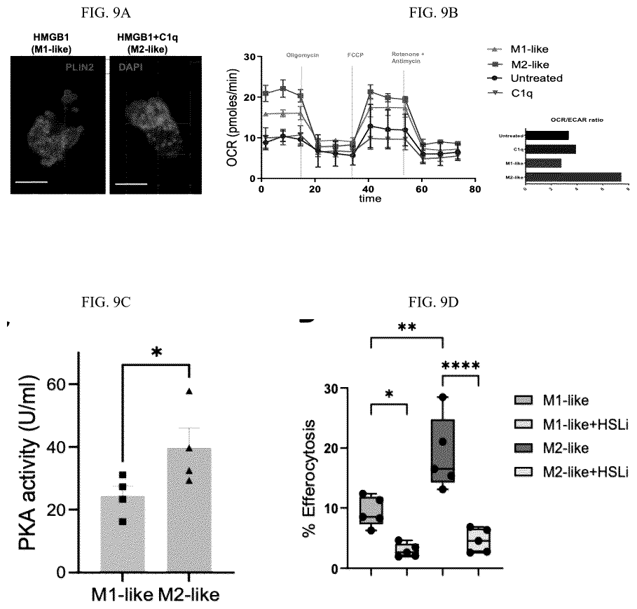

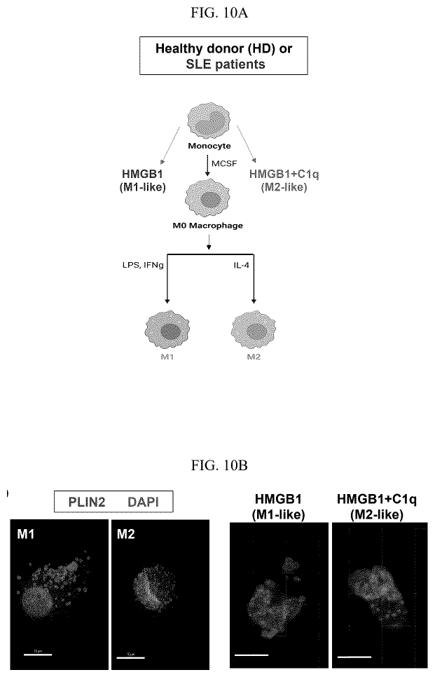

In some embodiments, upstream signaling pathways control HSL-mediated lipolysis. C1q binds HMGB1, activating PKA, and HSL inhibition blocs the efferocytosis. In some embodiments, the physiological relevance of HMGB1 and C1q in enhancing M2 macrophage polarization and efferocytosis, as well as the method for activating the entire pathway are provided. FIGS. 9A-9D demonstrate that C1q reverses HMGB1-associated macrophage dysfunction through a PKA-HSL-dependent lipolytic pathway. In some embodiments, human monocyte-derived macrophages are polarized to an inflammatory macrophage state by exposure to HMGB1 and to a pro-resolving macrophage state by exposure to HMGB1 in combination with C1q . In some embodiments, FIG. 9A shows that HMGB1-treated macrophages exhibit enlarged lipid droplets, whereas macrophages exposed to HMGB1 and C1q exhibit smaller lipid droplets, thereby indicating that C1q promotes lipid droplet remodeling toward a pro-resolving phenotype. In some embodiments, FIG. 9B shows that macrophages exposed to HMGB1 and C1q exhibit increased basal and maximal oxygen consumption rate relative to HMGB1-treated macrophages, thereby indicating improved mitochondrial respiratory capacity and metabolic fitness. In some embodiments, FIG. 9C shows that macrophages exposed to HMGB1 and C1q exhibit increased protein kinase A activity relative to HMGB1-treated macrophages. In some embodiments, FIG. 9D shows that macrophages exposed to HMGB1 and C1q exhibit increased efferocytosis, and that such restored efferocytosis is abrogated by inhibition of hormone-sensitive lipase, thereby indicating that the C1q-mediated functional rescue is dependent on HSL activity. In some embodiments, FIGS. 9A-9D collectively indicate that C1q opposes HMGB1-induced lipid droplet accumulation, restores oxidative metabolism, increases PKA signaling, and promotes HSL-dependent efferocytosis in macrophages. In some embodiments, these findings support use of C1q, or an agent that activates a corresponding PKA-HSL lipolytic pathway, to restore pro-resolving macrophage function in a disease or condition associated with impaired efferocytosis, inflammatory macrophage polarization, dysregulated lipid metabolism, or a combination thereof.

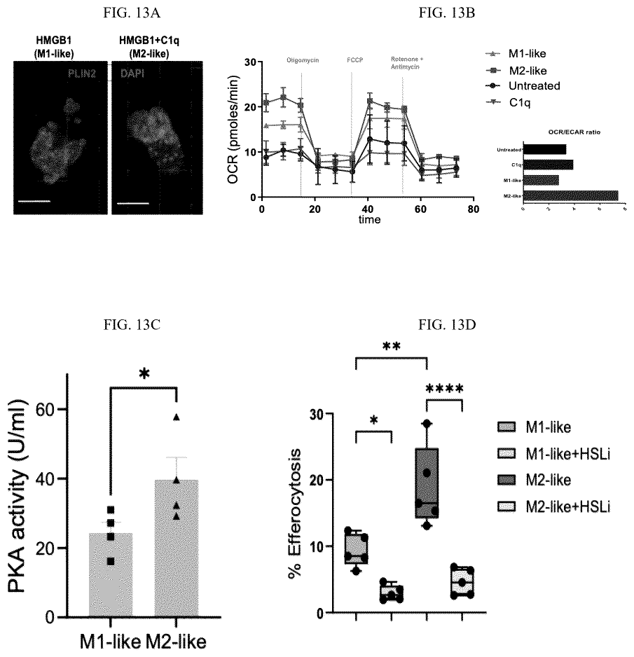

In some embodiments, FIGS. 13A-13D show that exposure to HMGB1 increases lipid droplet accumulation and impairs metabolic fitness and efferocytosis, whereas co-exposure to C1q reduces lipid droplet size, increases oxygen consumption rate and oxygen consumption rate to extracellular acidification rate ratio, increases protein kinase A activity, and restores efferocytosis in a hormone-sensitive lipase-dependent manner. In some embodiments, FIGS. 13A-13D indicate that C1q reverses HMGB1-associated macrophage dysfunction through a protein kinase A-hormone-sensitive lipase-regulated lipolytic pathway.

In some embodiments, a macrophage exposed to HMGB1 in combination with C1q exhibits a phenotype resembling an IL-4-induced M2 macrophage. In some embodiments, such phenotype includes reduced lipid droplet accumulation, reduced lipid droplet size, increased oxygen consumption rate, an increased oxygen consumption rate to extracellular acidification rate ratio, increased protein kinase A activity, and increased efferocytosis relative to a macrophage exposed to HMGB1 alone. In some embodiments, inhibition of hormone-sensitive lipase decreases or abolishes the efferocytic capacity of the HMGB1- and C1q-exposed macrophage, thereby indicating that hormone-sensitive lipase activity contributes to establishment or maintenance of a pro-resolving M2-like macrophage state.

In some embodiments, reduced C1q activity, C1q deficiency, increased HMGB1 abundance, increased HMGB1 activity, or loss of coordinated C1q- HMGB1 signaling contributes to impaired pro-resolving or M2-like macrophage polarization in systemic lupus erythematosus. In some embodiments, HMGB1 in combination with C1q regulates macrophage lipid metabolism, including lipid droplet breakdown, and directs differentiation, maintenance, or restoration of a pro-resolving or M2-like macrophage phenotype. In some embodiments, reduced M2-like macrophage function associated with reduced C1q or excess HMGB1 contributes to defective efferocytosis, persistence of apoptotic material, and enhanced inflammation in a subject having systemic lupus erythematosus. In some embodiments, treatment includes increasing C1q activity, decreasing HMGB1 activity, restoring coordinated C1q-HMGB1 signaling, or a combination thereof to improve lipid droplet remodeling, macrophage homeostasis, and efferocytosis. In some embodiments, such pathways identify a macrophage function that is maladaptive in systemic lupus erythematosus and is amenable to therapeutic correction.

In some embodiments, a therapeutic strategy for systemic lupus erythematosus includes combining an anti-inflammatory agent with an agent that restores lipid remodeling, mitochondrial metabolism, membrane remodeling, efferocytosis receptor organization, or a combination thereof in a macrophage. In some embodiments, such combination treatment improves clearance of apoptotic cells and promotes resolution of inflammation in a subject having systemic lupus erythematosus, lupus nephritis, or a related autoimmune condition.

### PDE3A Inhibition, cAMP-PKA-HSL Signaling, and microRNA Regulation

In some embodiments, intracellular cyclic adenosine monophosphate (cAMP) signaling is increased by decreasing phosphodiesterase 3A (PDE3A) activity or expression. In some embodiments, decreasing PDE3A activity increases cAMP, thereby increasing PKA activity and HSL activation. In some embodiments, a PDE3 inhibitor includes milrinone, cilostazol, or a combination thereof. In some embodiments, PDE3A expression is reduced by one or more microRNAs, including miR-449c-5p, miR-6824-3p, miR-548av-5p, or a combination thereof.

As used herein, the term “PDE3A” refers to phosphodiesterase 3A. As used herein, the phrase “decreases phosphodiesterase 3A activity or expression” refers to reducing enzymatic activity, transcript abundance, protein abundance, or functional signaling attributable to phosphodiesterase 3A. As used herein, the term “phosphodiesterase 3 inhibitor” or “PDE3 inhibitor” refers to an agent that inhibits phosphodiesterase 3 activity and includes, in some embodiments, milrinone, cilostazol, or a combination thereof. As used herein, the term “microRNA that decreases phosphodiesterase 3A expression” refers to a microRNA that directly or indirectly reduces phosphodiesterase 3A transcript level, translation, protein abundance, or functional expression.

In some embodiments, comparative transcript profiling, microRNA profiling, or integrated microRNA-mRNA network analysis is used to distinguish an inflammatory macrophage state from a pro-resolving macrophage state. In some embodiments, a macrophage exposed to HMGB1 in combination with C1q exhibits reduced phosphodiesterase 3A transcript abundance relative to a macrophage exposed to HMGB1 alone. In some embodiments, the macrophage exposed to HMGB1 in combination with C1q further exhibits increased abundance of one or more microRNAs predicted to decrease phosphodiesterase 3A expression, including miR-449c-5p, miR-6824-3p, miR-548av-5p, or a combination thereof. In some embodiments, reduced phosphodiesterase 3A expression decreases hydrolysis of cyclic adenosine 3′,5′-monophosphate, thereby increasing protein kinase A activity, increasing hormone-sensitive lipase activation, promoting lipid droplet lipolysis, and enhancing efferocytosis. In some embodiments, the disclosed microRNA-phosphodiesterase 3A-protein kinase A pathway is used to identify, induce, maintain, or restore a pro-resolving or M2-like macrophage phenotype.

In some embodiments, a method of increasing macrophage efferocytosis or promoting a pro-resolving macrophage phenotype includes administering to a cell, tissue, or subject one or more microRNAs that decrease phosphodiesterase 3A expression, or an agent that increases abundance or activity of the one or more microRNAs, thereby decreasing phosphodiesterase 3A expression, increasing cyclic adenosine 3′,5′-monophosphate signaling, increasing protein kinase A activity, increasing hormone-sensitive lipase activation, promoting lipid droplet remodeling, and enhancing efferocytosis. In some embodiments, the one or more microRNAs include miR-449c-5p, miR-6824-3p, miR-548av-5p, or a combination thereof. In some embodiments, the method is used to treat systemic lupus erythematosus, lupus nephritis, chronic inflammation, autoimmunity, or a related inflammatory disorder. In some embodiments, the one or more microRNAs are administered alone or in combination with a phosphodiesterase 3 inhibitor, C1q , nervonic acid, a beta-adrenergic receptor agonist, or a combination thereof.

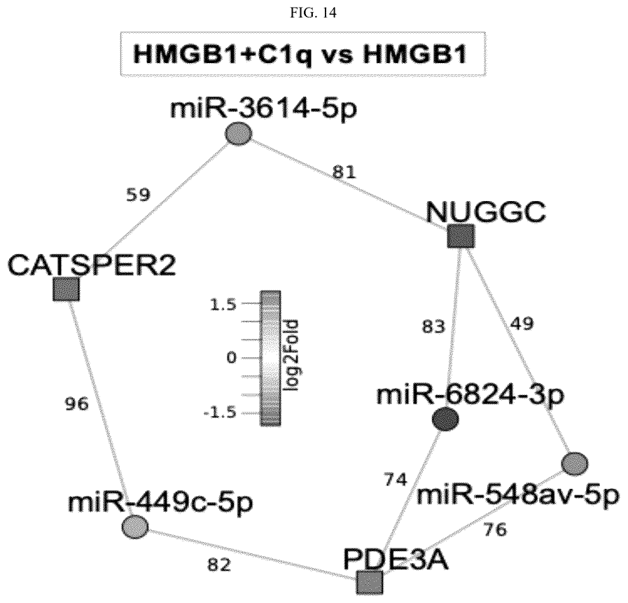

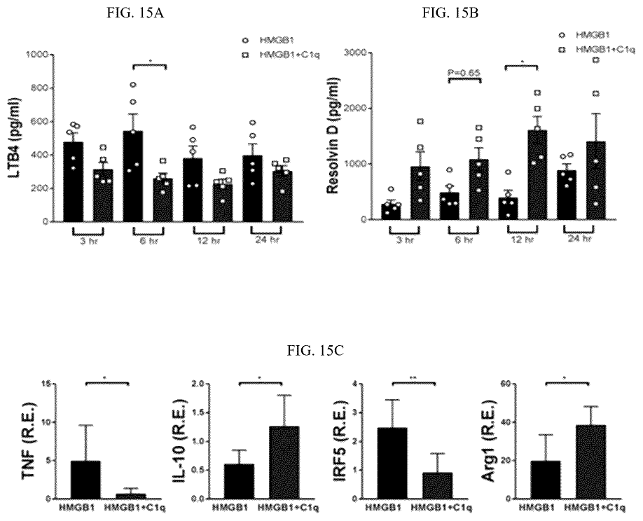

In some embodiments, FIG. 14 shows an integrated microRNA-mRNA network associated with HMGB1- and C1q-regulated macrophage programming. In some embodiments, decreased phosphodiesterase 3A expression in macrophages exposed to HMGB1 and C1q is associated with increased abundance of one or more microRNAs predicted to target phosphodiesterase 3A, thereby linking microRNA-mediated reduction of phosphodiesterase 3A to increased cyclic adenosine 3′,5′-monophosphate-protein kinase A-hormone-sensitive lipase signaling and enhanced efferocytosis.

### Adrenergic Activation and Neuroimmune Stimulation

In some embodiments, lipase activation is achieved through adrenergic signaling. In some embodiments, the adrenergic signal includes norepinephrine, a beta-adrenergic receptor agonist, a beta2-adrenergic receptor agonist, or a combination thereof. In some embodiments, the agonist includes isoproterenol. In some embodiments, neuroimmune stimulation is used to promote adrenergic activation of cAMP-PKA-HSL signaling in macrophages. In some embodiments, the neuroimmune stimulation includes vagus nerve stimulation. In some embodiments, such activation increases efferocytosis, promotes a pro-resolving macrophage phenotype, increases lipid droplet remodeling, increases membrane remodeling, increases mitochondrial oxidative metabolism, increases serial engulfment capacity, or combinations thereof.

As used herein, the term “adrenergic activation” refers to activation of adrenergic signaling sufficient to increase intracellular cyclic adenosine monophosphate signaling, protein kinase A activity, hormone-sensitive lipase activity, or a combination thereof. As used herein, the term “beta-adrenergic receptor agonist” refers to an agent that activates one or more beta-adrenergic receptors. As used herein, the term “beta2-adrenergic receptor agonist” refers to an agent that activates a beta2-adrenergic receptor. As used herein, the term “vagus nerve stimulation” refers to stimulation of the vagus nerve by electrical, mechanical, physiological, pharmacologic, or other neuroimmune stimulation sufficient to promote adrenergic activation in macrophages in some embodiments.

In some embodiments, neuroimmune stimulation is used to activate a cAMP-PKA-HSL pathway in a macrophage, monocyte, or subject in need thereof. In some embodiments, the neuroimmune stimulation includes vagus nerve stimulation. In some embodiments, vagus nerve stimulation promotes release of one or more neurotransmitters that activate adrenergic signaling in a macrophage, thereby increasing cyclic adenosine monophosphate signaling, protein kinase A activity, phosphorylation of hormone-sensitive lipase, lipid droplet lipolysis, or a combination thereof. In some embodiments, the neurotransmitter includes norepinephrine. In some embodiments, the adrenergic signaling includes signaling through one or more beta-adrenergic receptors, including a beta2-adrenergic receptor. In some embodiments, a beta-adrenergic receptor agonist, a beta2-adrenergic receptor agonist, norepinephrine, isoproterenol, or a combination thereof is used to increase HSL activity and promote a pro-resolving macrophage phenotype. In some embodiments, such activation increases efferocytosis, increases serial engulfment capacity, reduces lipid droplet accumulation or size, increases mitochondrial respiration, increases membrane fluidity, promotes receptor organization at an apoptotic cell contact site, or a combination thereof. In some embodiments, vagus nerve stimulation or adrenergic activation is used to treat a disease or condition associated with impaired macrophage efferocytosis, inflammatory macrophage polarization, dysregulated lipid metabolism, or a combination thereof. In some embodiments, the disease or condition includes systemic lupus erythematosus, lupus nephritis, chronic inflammation, autoimmunity, or a related inflammatory disorder. In some embodiments, an HSL agonist is identified by screening one or more neurotransmitters, adrenergic agonists, or neuroimmune stimulation conditions for the ability to increase HSL phosphorylation, HSL localization to lipid droplets, mitochondrial oxidative metabolism, efferocytosis, or a combination thereof.

In some embodiments, neuroimmune adrenergic signaling is used to enhance macrophage efferocytosis in a subject having systemic lupus erythematosus or lupus nephritis. In some embodiments, the adrenergic signaling includes norepinephrine, a beta-adrenergic receptor agonist, a beta2-adrenergic receptor agonist, or a combination thereof. In some embodiments, the adrenergic signaling activates cyclic adenosine monophosphate signaling and protein kinase A, thereby increasing phosphorylation and activation of hormone-sensitive lipase and promoting lipid droplet remodeling in a macrophage. In some embodiments, activation of the beta2-adrenergic receptor increases macrophage efferocytosis, increases serial engulfment capacity, reduces lipid droplet accumulation or size, increases mitochondrial respiration, promotes membrane remodeling, or a combination thereof. In some embodiments, the macrophage is a human monocyte-derived macrophage, a mouse macrophage, a monocyte, a tissue macrophage, or a macrophage derived from a subject having systemic lupus erythematosus. In some embodiments, the effect of norepinephrine or a beta2-adrenergic receptor agonist is assessed in the presence or absence of a beta2-adrenergic receptor antagonist, a protein kinase A inhibitor, a hormone-sensitive lipase inhibitor, or a combination thereof, thereby determining whether enhancement of efferocytosis is mediated through a beta2-adrenergic receptor-protein kinase A-hormone-sensitive lipase pathway. In some embodiments, such adrenergic activation restores a pro-resolving macrophage phenotype in a lupus-associated inflammatory setting.

Further embodiments are directed to lipase modulators that are lipase inhibitors, such as but not limited to Atglistatin (an ATGL inhibitor), HSL-IN-1 (an HSL inhibitor), and MJN110 (a MAGL inhibitor), where such lipase inhibitors or compositions including such lipase inhibitors, reduce (i.e., impair, inhibit, suppress) efferocytosis, increase LD size, reduce LD density, produce a pro-inflammatory M1 macrophage profile, reduce the number of macrophage surface M2 markers, enhance anti-tumor immunity, reduce tumor-producing effects, or combinations thereof. In some objects, lipase inhibitors, such as but not limited to HSL inhibitors, suppress tumors, restores anti-tumor immunity, sensitizes cancer cells, improves the effectiveness of chemotherapy, immunotherapy, radiation therapy, or combinations thereof.

In some embodiments, a method of modulating a tumor-associated macrophage includes contacting the tumor-associated macrophage, or administering to a subject having cancer a composition including a lipase inhibitor, thereby altering macrophage lipid metabolism in a tumor microenvironment. In some embodiments, the lipase inhibitor includes an adipose triglyceride lipase inhibitor, a hormone-sensitive lipase inhibitor, a monoglyceride lipase inhibitor, a lysosomal acid lipase inhibitor, or a combination thereof. In some embodiments, the method reduces a macrophage activity that supports tumor persistence, tumor growth, immune suppression, tissue remodeling, or a combination thereof. In some embodiments, the composition is administered alone or in combination with an anti-cancer therapy, an immune-modulating therapy, or a combination thereof. In some embodiments, the tumor-associated macrophage is monitored by assessing one or more of lipid droplet burden, inflammatory marker expression, scavenger receptor expression, phagocytic activity, or combinations thereof.

In some embodiments, the disclosure provides therapeutic applications of targeted activation or inhibition of lipases in macrophages.

### Lipid Droplet (LD) Remodeling and Lipid Modulators

Characteristics of lipid droplets (LDs) influence macrophage function. In some embodiments, methods of stimulating cells, such as monocytes, to activate intracellular signaling pathways that lead to the expression of specific genes and proteins attributed to M1 macrophages, increases the total LD volume in M1 macrophages, produce inflammatory cytokines and reactive oxygen species, suppresses abnormal tissue growth (tumor: cancerous, non-cancerous, or precancerous), increases antimicrobial defenses, or combinations thereof. Stimulation of cells towards M1 macrophages occurs with, for example, pro-inflammatory T helper 1 (Th1) cytokines (e.g., interferon-gamma (IFN-γ), tumor necrosis factor alpha (TNFα), interleukin-12 (IL-12)), or molecules or microbial products that induce Th1 cytokine production (e.g., lipopolysaccharide (LPS), pathogen-associated molecular pattern (PAMP), damage-associated molecular pattern (DAMP)).

In other embodiments, methods of stimulating cells, such as monocytes, to express specific genes and proteins attributed to M2 macrophages, increase the quantity of LDs in M2 macrophages, produce anti-inflammatory cytokines, increase expression of CD206, MERTK, and PPARγ, increases efferocytosis, increases lipase activity, or combinations thereof. Stimulation of cells towards M2 macrophages occurs with, for example, anti-inflammatory T helper 2 (Th2) cytokines (e.g., interleukin-4 (IL-4), IL-5, IL-10), or molecules that induce Th2 cytokine production (e.g., microRNAs (miRNAs), long non-coding RNAs (lncRNAs), metabolic factors (e.g., hypoxia, lactate production), activation of the Notch signaling pathway (e.g., using ligands (e.g., DDL4), activation of the NF-κB p50 subunit).

Macrophage lipid droplets (LDs) function as energy reservoirs but also as regulators of cytokine production and efferocytosis. In some embodiments, LD lipolysis modulators are useful in treating diseases associated with dysregulated macrophage function. Nonlimiting examples of such diseases include autoimmune diseases and cancers or malignancies.

As used herein, the term “lipid droplet” refers to an intracellular organelle including neutral lipids and associated proteins, including an organelle involved in energy storage, lipid metabolism, membrane precursor supply, or signaling. As used herein, the term “lipid droplet remodeling” refers to a change in lipid droplet number, size, density, composition, turnover, localization, or associated protein content.

In some embodiments, intracellular lipid droplets have a beneficial function in a macrophage when organized, stored, and remodeled in a manner that supports cellular homeostasis, efferocytosis, and controlled lipid mobilization. In some embodiments, a therapeutically beneficial lipid droplet state is distinguished from a maladaptive lipid droplet state by one or more of increased compatibility with lipase-dependent remodeling, increased oxidative metabolism, increased mitochondrial fitness, increased membrane remodeling, increased efferocytosis, or increased expression of one or more pro-resolving markers. In some embodiments, a disclosed method provides a mechanism-based therapeutic approach by manipulating lipid droplet size, number, composition, dispersal, turnover, lipolysis, or a combination thereof in order to restore macrophage homeostasis. In some embodiments, the disclosed methods modulate intracellular lipid droplets to promote a beneficial macrophage function rather than merely reducing total intracellular lipid accumulation.

In some embodiments, macrophage lipid droplet-associated phenotypes are identified or modulated based on lipid composition. In some embodiments, a pro-resolving macrophage phenotype includes increased phosphatidylinositol species, increased lysophosphatidylinositol species, increased nervonic acid, or combinations thereof. In some embodiments, the phosphatidylinositol species include PI(18:0_18:0), PI(16:0/20:4), or both. In some embodiments, an inflammatory macrophage phenotype includes increased ceramide, increased cholesterol, increased triacylglycerol, increased phosphatidylethanolamine, or combinations thereof. In some embodiments, inhibition of HSL shifts a macrophage lipid profile from a pro-resolving lipid signature toward an inflammatory lipid signature. In some embodiments, such lipid signatures are used to identify macrophage state, monitor treatment response, stratify a subject, or screen an agent for pro-resolving activity.

As used herein, the term “lipid droplet composition” refers to the identity, amount, relative abundance, distribution, or combination of one or more lipid species present in, stored in, associated with, or characteristic of one or more lipid droplets in a cell, including a macrophage. In some embodiments, lipid droplet composition includes the presence, level, or relative abundance of one or more neutral lipids, fatty acids, phospholipids, lysophospholipids, sterols, ceramides, triacylglycerols, phosphatidylinositol species, lysophosphatidylinositol species, or combinations thereof. As used herein, the term “phosphatidylinositol species” refers to a phosphatidylinositol molecular species associated with macrophage state, including PI(18:0_18:0) in some embodiments.

In some embodiments, a pro-resolving macrophage population is identified in a biological sample based on a lipid signature associated with enhanced efferocytosis and lipid droplet remodeling. In some embodiments, the biological sample includes blood, peripheral blood mononuclear cells, isolated monocytes, macrophages, tissue, lavage fluid, biopsy material, or a combination thereof. In some embodiments, identifying the pro-resolving macrophage population includes detecting an increased level of nervonic acid, a phosphatidylinositol species, a lysophosphatidylinositol species, or a combination thereof relative to a reference level. In some embodiments, the phosphatidylinositol species includes PI(18:0_18:0), PI(16:0/20:4), or both. In some embodiments, the reference level is obtained from a healthy control, an inflammatory macrophage population, an untreated sample, a baseline sample from the same subject, or a combination thereof. In some embodiments, the detected lipid signature is used to identify macrophage state, stratify a subject, monitor treatment response, evaluate disease activity, or select a subject for treatment with a lipase modulator, nervonic acid, a PDE3 inhibitor, an adrenergic agonist, C1q , or a combination thereof. As used herein, the term “biological sample” refers to any sample including cells, tissue, fluid, or a component thereof that is suitable for detection of a biomarker disclosed herein. As used herein, the term “reference level” refers to a comparator level obtained from a control sample, a healthy sample, a baseline sample, an untreated sample, a non-pro-resolving sample, or another suitable reference.

In some embodiments, a subject is selected for treatment with a lipase modulator, nervonic acid, C1q , a phosphodiesterase 3 inhibitor, a beta-adrenergic receptor agonist, or a combination thereof based on a macrophage-associated lipid signature indicative of impaired efferocytosis or a pro-resolving macrophage state. In some embodiments, the lipid signature includes one or more of nervonic acid abundance, ceramide abundance, phosphatidylinositol 18:0_18:0 abundance, lysophosphatidylinositol 18:0 abundance, lipid droplet number, lipid droplet size, total lipid droplet burden, or combinations thereof. In some embodiments, the lipid signature is measured in a biological sample, a macrophage preparation, or a tissue sample from the subject. In some embodiments, the lipid signature is used to stratify the subject, identify a target macrophage population, monitor treatment response, or select combination therapy.

In some embodiments, lipid droplet composition in a macrophage is a determinant of efferocytic activity. In some embodiments, a macrophage having a lipid droplet composition associated with impaired lipid mobilization, impaired membrane remodeling, or impaired pro-resolving activity exhibits reduced efferocytosis relative to a macrophage having a lipid droplet composition associated with a pro-resolving state. In some embodiments, efferocytosis is increased by altering lipid droplet composition, lipid droplet remodeling, lipid droplet dispersal, lipid droplet lipolysis, or a combination thereof. In some embodiments, a disclosed method includes contacting a macrophage with a lipid supplement, a fatty acid, a phospholipid, a lipase modulator, or a combination thereof in an amount effective to alter lipid droplet composition and thereby improve efferocytosis.

In some embodiments, lipid supplementation is used to alter intracellular lipid pools in a macrophage, including an M2 macrophage, an M2-like macrophage, or both. In some embodiments, the supplemented lipid includes a fatty acid, a phospholipid, a lysophospholipid, a neutral lipid precursor, or a combination thereof. In some embodiments, the supplemented lipid is stored in one or more lipid droplets and subsequently mobilized to support membrane remodeling, mitochondrial metabolism, lipid catabolism, efferocytosis, or a combination thereof. In some embodiments, defects in lipid storage, lipid droplet organization, or lipid catabolism are associated with a disease or condition including obesity, cardiovascular disease, type 2 diabetes, systemic lupus erythematosus, or a combination thereof. In some embodiments, the effect of lipid supplementation is assessed by measuring one or more of lipid droplet abundance, lipid droplet composition, fatty acid abundance, membrane fluidity, mitochondrial respiration, macrophage polarization, or efferocytosis. In some embodiments, lipid supplementation preferentially expands, restores, or remodels lipid pools associated with an M2 or M2-like macrophage phenotype.

### Nervonic Acid, Ceramide Metabolism, and Reparative Lipid Programming

In some embodiments, nervonic acid, also referred to herein as NA or FA 24:1, is a very long-chain monounsaturated fatty acid useful for modulating macrophage function, lipid metabolism, efferocytosis, or combinations thereof. In some embodiments, nervonic acid is enriched in anti-inflammatory or pro-resolving macrophages relative to inflammatory macrophages. In some embodiments, nervonic acid is a lipid droplet metabolism-associated modulator that enhances macrophage efferocytosis, promotes a pro-resolving phenotype, increases mitochondrial respiration, increases oxidative metabolism, increases PPARγ-associated activity or expression, improves membrane fluidity, facilitates receptor organization involved in apoptotic cell uptake, or combinations thereof. In some embodiments, nervonic acid is administered exogenously to a macrophage, monocyte, or subject in need thereof to restore defective efferocytosis. In some embodiments, nervonic acid functions in a pathway associated with adipose triglyceride lipase (ATGL)-and/or hormone-sensitive lipase (HSL)-regulated macrophage lipid remodeling, while not necessarily directly activating or inhibiting a lipase. In some embodiments, nervonic acid is generated or increased in association with ceramide metabolism, including through acid ceramidase activity, such as ASAH1 activity.

As used herein, the term “nervonic acid” refers to the very long-chain monounsaturated fatty acid FA 24:1, including preparations, conjugates, and formulations thereof suitable for use in the disclosed compositions and methods.

In some embodiments, nervonic acid is associated with an acid ceramidase pathway. In some embodiments, acid ceramidase includes N-acylsphingosine amidohydrolase 1 (ASAH1). In some embodiments, a pro-resolving macrophage phenotype is associated with increased ASAH1 expression, increased ASAH1 secretion, decreased ceramide species, including ceramide (d18:1/24:1), increased nervonic acid abundance, or combinations thereof. In some embodiments, nervonic acid is generated or increased in association with ceramide turnover mediated by ASAH1. As used herein, the term “ASAH1” refers to N-acylsphingosine amidohydrolase 1, also referred to in some embodiments as acid ceramidase. As used herein, the term “ceramidase” or “CDase” refers to an enzyme that hydrolyzes ceramide and includes acid ceramidase in some embodiments.

In some embodiments, a method of treating a disease or condition associated with impaired macrophage efferocytosis, dysregulated ceramide metabolism, altered lipid droplet remodeling, or a combination thereof includes administering to a subject in need thereof an agent that increases N-acylsphingosine amidohydrolase 1 activity, expression, secretion, or a combination thereof, thereby increasing nervonic acid-associated lipid remodeling, promoting a pro-resolving macrophage phenotype, and increasing macrophage efferocytosis. In some embodiments, the disease or condition includes systemic lupus erythematosus, lupus nephritis, chronic inflammation, autoimmunity, a neuroinflammatory disease, a neurodegenerative disease, or a combination thereof. In some embodiments, the agent that increases N-acylsphingosine amidohydrolase 1 activity is administered alone or in combination with nervonic acid, C1q , a phosphodiesterase 3 inhibitor, a beta-adrenergic receptor agonist, or a combination thereof. In some embodiments, treatment efficacy is assessed by one or more of efferocytosis, lipid droplet burden, acid ceramidase abundance, nervonic acid abundance, PPARγ expression, mitochondrial respiration, membrane fluidity, renal inflammation, proteinuria, blood urea nitrogen, creatinine, anti-dsDNA, or combinations thereof.

In some embodiments, acid ceramidase 1 hydrolyzes ceramide to generate sphingosine and a free fatty acid. In some embodiments, acid ceramidase 1 activity is associated with generation of nervonic acid, a long-chain monounsaturated fatty acid, such as C24:1. In some embodiments, nervonic acid is enriched in reparative or pro-resolving macrophages and is associated with increased mitochondrial function, improved membrane properties, and enhanced efferocytosis. In some embodiments, acid ceramidase 1 expression is increased in macrophages exhibiting a reparative phenotype.

In some embodiments, exogenous nervonic acid is administered to macrophages or a subject in need thereof to restore defective macrophage function. In some embodiments, nervonic acid treatment increases mitochondrial respiration, increases oxidative metabolism, improves membrane fluidity, promotes receptor organization at the phagocytic synapse, and rescues impaired efferocytosis under inflammatory conditions. In some embodiments, nervonic acid rescues defective efferocytosis in macrophages obtained from a lupus-prone subject or lupus-prone animal model. In some embodiments, the disclosed methods further include assessing acid ceramidase 1 dependence of the reparative lipid program and determining whether restoration of macrophage function is associated with mitochondrial remodeling, membrane remodeling, or both.

In some embodiments, the disclosed methods establish a reparative lipid circuit linking complement-associated signaling, damage-associated molecular pattern signaling, lipid droplet lipolysis, acid ceramidase 1 activity, nervonic acid availability, mitochondrial metabolism, and macrophage efferocytosis. In some embodiments, modulation of one or more components of this circuit is used to restore immune homeostasis, reduce inflammatory tissue injury, and treat systemic lupus erythematosus, lupus nephritis, or a related autoimmune condition

In some embodiments, macrophages associated with systemic lupus erythematosus exhibit heterogeneous defects that affect the extent to which nervonic acid supplementation restores efferocytosis. Such defects can include impaired lipid uptake, altered intracellular lipid trafficking, defective phagocytic synapse assembly, reduced membrane phosphoinositide competence, impaired mitochondrial fitness, altered membrane fluidity, or combinations thereof. In some embodiments, where nervonic acid supplementation does not fully restore efferocytosis, the disclosed methods further include determining whether nervonic acid primarily improves mitochondrial function, membrane properties, or both. Mitochondrial function can be evaluated by mitochondrial oxygen consumption rate, oxidative metabolism, reactive oxygen species production, or combinations thereof. Membrane properties can be evaluated by membrane fluidity, membrane lipid composition, receptor clustering, phosphatidylinositol and phosphatidylinositol-3-phosphate-associated signaling, or combinations thereof.

In some embodiments, nervonic acid is administered in combination with one or more additional agents that enhance macrophage pro-resolving function. Such additional agents can include a peroxisome proliferator-activated receptor gamma agonist, an activator of protein kinase A, an activator of hormone-sensitive lipase, or a combination thereof. In some embodiments, such combination treatment is used to restore phosphoinositide or phosphatidylinositol-3-phosphate competence at the plasma membrane, improve phagocytic synapse assembly, improve lipid remodeling, increase oxidative metabolism, or enhance efferocytosis.

In some embodiments, exogenous nervonic acid is administered to macrophages at about 1 μM to about 50 μM. In some embodiments, exogenous nervonic acid is administered at about 10 μM. In some embodiments, macrophages are contacted with nervonic acid for about 24 hours. In some embodiments, nervonic acid is provided as a protein conjugate, including a bovine serum albumin-conjugated preparation or a human serum albumin-conjugated preparation. In some embodiments, exogenous nervonic acid increases macrophage efferocytosis, increases PPARγ expression, increases basal respiration, increases maximal respiration, increases ATP production, increases spare respiratory capacity, or combinations thereof. In some embodiments, higher concentrations may be avoided where lipid toxicity is observed.

In some embodiments, a pharmaceutical composition includes nervonic acid, or a pharmaceutically acceptable salt thereof, and one or more pharmaceutically acceptable excipients. In some embodiments, nervonic acid is formulated for administration as a free fatty acid, as a pharmaceutically acceptable salt, as a complex with a carrier protein, or as part of a delivery system suitable for increasing availability to a macrophage or monocyte. In some embodiments, nervonic acid is conjugated to serum albumin, including bovine serum albumin or human serum albumin. In some embodiments, the nervonic acid composition is formulated as a solution, dispersion, emulsion, suspension, liposomal preparation, micellar preparation, nanoparticle formulation, or injectable formulation. In some embodiments, the nervonic acid composition is formulated for intraperitoneal, intravenous, oral, subcutaneous, or local administration. In some embodiments, the nervonic acid composition is administered in an amount effective to increase macrophage efferocytosis, increase mitochondrial respiration, increase oxidative metabolism, increase PPARγ-associated activity, improve membrane fluidity, or a combination thereof.

As used herein, the term “pharmaceutically acceptable salt,” when used with respect to nervonic acid or another disclosed agent, refers to a salt form that is suitable for administration to a subject and retains the intended biological activity of the parent compound.

In some embodiments, where systemic delivery of nervonic acid provides limited renal bioavailability or undesired off-target distribution, the delivery regimen is modified by dose titration, altered dosing frequency, altered route of administration, formulation in a lipid nanoparticle, formulation in a carrier suitable for renal targeting, or combinations thereof. In some embodiments, kidney macrophage endpoints are used to optimize such delivery conditions.

In some embodiments, one or more additional anti-inflammatory lipid species are identified and used alone or in combination with nervonic acid in the disclosed methods. Suitable lipid species can include phosphatidylinositol 18:0_18:0, lysophosphatidylinositol 18:0, or other pro-resolving lipids identified by lipidomic analysis. In some embodiments, such lipid species are evaluated for their ability to improve macrophage efferocytosis, lipid droplet remodeling, mitochondrial function, membrane organization, inflammatory resolution, or combinations thereof.

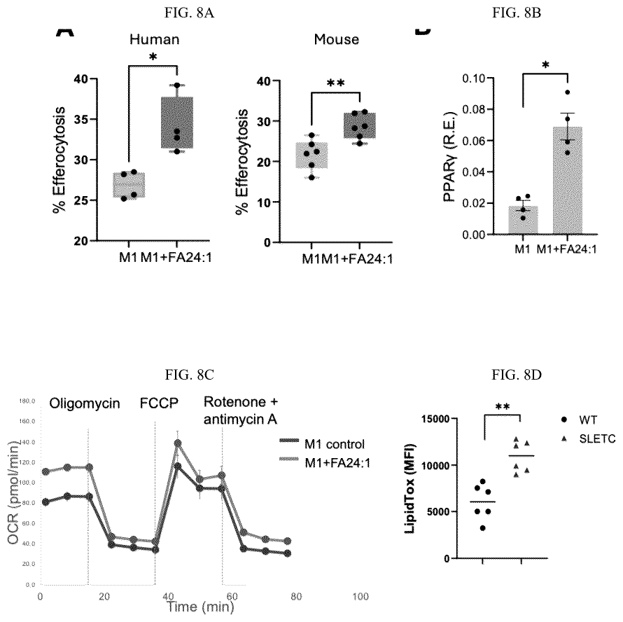

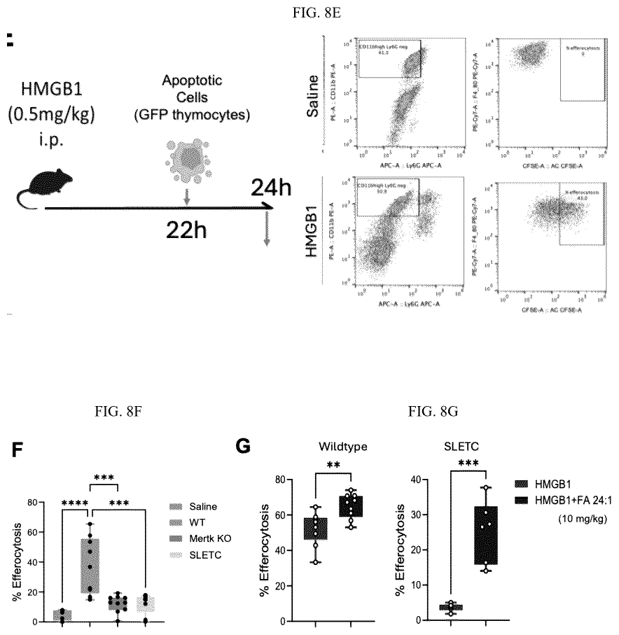

In some embodiments, FIGS. 8A-8G demonstrate that replenishing nervonic acid, also referred to herein as FA 24:1, restores pro-resolving macrophage function both in vitro and in vivo. In some embodiments, FIG. 8A shows that exogenous FA 24:1 treatment of inflammatory M1 macrophages rescues efferocytic capacity in human and mouse in vitro models, thereby indicating that nervonic acid can reverse impaired apoptotic cell clearance in inflammatory macrophages. In some embodiments, FIG. 8B demonstrates that such functional rescue is associated with an increase in PPARγ expression, thereby indicating that nervonic acid promotes a transcriptional program associated with a pro-resolving macrophage phenotype. In some embodiments, FIG. 8C presents Seahorse analysis showing that FA 24:1 treatment enhances mitochondrial respiratory capacity, including oxygen consumption rate, in M1 macrophages, thereby indicating that nervonic acid improves macrophage metabolic fitness associated with pro-resolving activity. In some embodiments, FIG. 8D demonstrates that macrophages from SLETC mice exhibit increased lipid accumulation relative to wild-type controls, thereby validating the SLETC model as a disease-relevant model of lipid-associated macrophage dysfunction and impaired efferocytosis. In some embodiments, FIG. 8E provides an in vivo framework for assessing macrophage efferocytosis, where the left panel shows a schematic of a sterile peritonitis model and the right panel shows representative flow plots identifying phagocytosed apoptotic cells, thereby establishing an assay system for measuring efferocytic function in vivo. In some embodiments, FIG. 8F shows that intraperitoneal HMGB1 impairs efferocytosis in wild-type mice, with Mertk knockout mice serving as a negative control, thereby validating the in vivo model as a system for detecting inflammatory suppression of efferocytosis. In some embodiments, FIG. 8G demonstrates that systemic administration of FA 24:1 restores efferocytosis in both HMGB1-treated wild-type mice and in SLETC mice, thereby showing that nervonic acid can rescue defective macrophage function in vivo in both induced and disease-relevant settings. In some embodiments, FIGS. 8A-8G collectively indicate that nervonic acid is useful for reversing inflammatory macrophage dysfunction, restoring pro-resolving macrophage activity, improving mitochondrial fitness, and treating diseases or conditions associated with impaired efferocytosis, dysregulated lipid metabolism, or both. In some embodiments, nervonic acid is administered in vivo to restore impaired macrophage efferocytosis in an inflammatory setting. In some embodiments, the inflammatory setting includes HMGB1-induced sterile peritonitis. In some embodiments, nervonic acid is administered intraperitoneally at about 10 mg/kg. In some embodiments, a single administration restores or increases efferocytosis in vivo. In some embodiments, the subject is lupus-prone. In some embodiments, the subject or animal model includes an SLETC mouse or an NZB/W F1 mouse. In some embodiments, repeated nervonic acid administration includes about 10 mg/kg intraperitoneally every other day for about 2 to about 6 weeks, optionally beginning at about 20 weeks of age. In some embodiments, treatment efficacy is assessed by one or more of proteinuria, blood urea nitrogen, creatinine, anti-dsDNA, renal histopathology, periodic acid-Schiff staining, immune complex deposition, kidney macrophage lipid droplet burden, ASAH1, PLIN2, PPARγ, or combinations thereof.

### Neuroinflammatory and Neurodegenerative Applications

In some embodiments, the disclosed pathways are applicable to neuroinflammatory or neurodegenerative conditions associated with lipid droplet accumulation in myeloid cells. In some embodiments, amyloid-beta increases lipid droplet accumulation in human monocytes or macrophages. In some embodiments, C1q decreases or reverses amyloid-beta-induced lipid droplet accumulation. In some embodiments, such embodiments are useful in neuroinflammatory disease, neurodegenerative disease, or both. In some embodiments, the degree of HSL dependence in such embodiments is further determined experimentally.

As used herein, the term “amyloid-beta” refers to an amyloid-beta peptide or aggregate associated with lipid droplet accumulation in a myeloid cell in some embodiments. As used herein, the term “myeloid cell” refers to a cell of the myeloid lineage, including a monocyte or macrophage.

In some embodiments, the disclosed pathways are useful in a neuroinflammatory or neurodegenerative disease associated with lipid droplet accumulation in a myeloid cell. In some embodiments, the myeloid cell includes a monocyte, macrophage, brain-associated macrophage, infiltrating myeloid cell, microglia-like cell, or a combination thereof. In some embodiments, amyloid-beta increases lipid droplet accumulation in a human monocyte or macrophage and thereby promotes an inflammatory or dysfunctional myeloid-cell state associated with impaired clearance activity. In some embodiments, C1q decreases or reverses amyloid-beta-induced lipid droplet accumulation, thereby promoting lipid droplet remodeling toward a more pro-resolving cellular phenotype. In some embodiments, the disclosed compositions and methods are useful for treating, preventing, reducing, delaying progression of, or monitoring a neuroinflammatory disease, a neurodegenerative disease, or both. In some embodiments, the neurodegenerative disease includes Alzheimer's disease. In some embodiments, a subject having a neuroinflammatory or neurodegenerative condition is selected for treatment based on evidence of lipid droplet accumulation, impaired myeloid-cell clearance activity, amyloid-beta-associated macrophage or monocyte dysfunction, or a combination thereof. In some embodiments, treatment includes administration of C1q, a lipase modulator, a PDE3 inhibitor, an adrenergic agonist, nervonic acid, or a combination thereof. In some embodiments, the effect of amyloid-beta, C1q, or both on lipid droplet remodeling is further evaluated for dependence on hormone-sensitive lipase activity, hormone-sensitive lipase phosphorylation, or both.

In some embodiments, a method of treating a neuroinflammatory or neurodegenerative disease associated with lipid droplet accumulation in a myeloid cell includes administering to a subject in need thereof C1q, a lipase modulator, nervonic acid, a phosphodiesterase 3 inhibitor, an adrenergic agonist, neuroimmune stimulation, or a combination thereof, thereby decreasing lipid droplet accumulation, promoting lipid droplet remodeling, increasing clearance activity, and promoting a pro-resolving myeloid-cell phenotype. In some embodiments, the neurodegenerative disease includes Alzheimer's disease. In some embodiments, the subject is selected for treatment based on evidence of lipid droplet accumulation, impaired myeloid-cell clearance activity, amyloid-beta-associated monocyte or macrophage dysfunction, or a combination thereof. In some embodiments, the effect of treatment is evaluated for dependence on hormone-sensitive lipase activity, hormone-sensitive lipase phosphorylation, or both.

Role of ATGL and HSL in Macrophage Function

Adipose triglyceride lipase (ATGL) and hormone-sensitive lipase (HSL) are exemplary enzymes involved in regulating lipid metabolism within macrophages. These enzymes play an important role in the hydrolysis of triglycerides (TGs) and the maintenance of lipid homeostasis. The activity of ATGL and HSL significantly affects the phenotype and function of macrophages, including their M1 (pro-inflammatory) and M2 (anti-inflammatory) polarization, inflammatory responses, phagocytic capacity, and overall contribution to disease pathology. Dysregulated lipid metabolism in macrophages, mediated by altered lipase activity, for example, ATGL and HSL activity, is associated in the development and progression of various diseases, such as but not limited to, atherosclerosis, non-alcoholic fatty liver disease, cancer, and Systemic Lupus Erythematosus (SLE) to name a few.

As used herein, the term “adipose triglyceride lipase” or “ATGL” refers to the lipase encoded by the PNPLA2 gene and involved in triglyceride hydrolysis. As used herein, the term “hormone-sensitive lipase” or “HSL” refers to the lipase encoded by the LIPE gene and includes active, phosphorylated, localized, translocated, endogenous, recombinant, or overexpressed forms thereof.

In some embodiments, HSL is modulated by genetic manipulation of LIPE, the gene encoding HSL. In some embodiments, reduced LIPE expression, including siRNA-mediated knockdown, decreases efferocytosis, decreases Annexin A1, decreases PPARγ, decreases CD36, decreases CD163, decreases CD200R1, decreases CD206, increases TNFα, increases IL1β, increases HIF1α, increases lipid droplet volume, or combinations thereof. In some embodiments, increased LIPE expression, including plasmid-mediated overexpression, increases efferocytosis, increases Annexin A1, increases PPARγ, increases CD36, increases CD163, increases CD206, increases fatty acid oxidation-associated gene expression, or combinations thereof. As used herein, the term “LIPE” refers to the gene encoding hormone-sensitive lipase.

In some embodiments, genetic modulation of LIPE is used to evaluate the role of hormone-sensitive lipase with reduced confounding from off-target effects associated with pharmacologic lipase inhibition. In some embodiments, reduction of LIPE expression in a macrophage, including by siRNA-mediated knockdown, decreases efferocytosis, decreases Annexin A1 secretion, increases TNFα production, and decreases expression of one or more markers associated with a pro-resolving or M2-like macrophage phenotype, including PPARγ, CD36, CD200R1, CD163, CD206, or a combination thereof. In some embodiments, increased LIPE expression in a macrophage, including plasmid-mediated overexpression, increases efferocytosis, increases Annexin A1 secretion, reduces TNFα production, reduces IL-1β production, and increases expression of one or more M2-associated markers including PPARγ, CD36, CD200R1, CD163, CD206, or a combination thereof. In some embodiments, such findings indicate that hormone-sensitive lipase is a regulator of macrophage polarization, inflammatory tone, and pro-resolving macrophage activity.

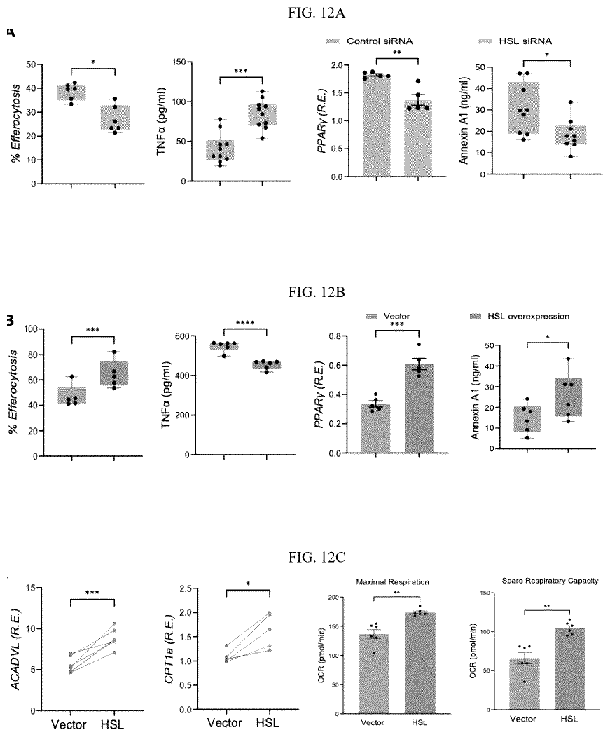

In some embodiments, FIGS. 12A-12C show that genetic reduction of LIPE decreases efferocytosis and Annexin A1 secretion and increases inflammatory cytokine production, whereas increased LIPE expression increases efferocytosis, increases Annexin A1 secretion, reduces TNFα and IL-1β, increases expression of M2-associated markers including PPARγ, CD36, and CD200R1, increases expression of fatty acid oxidation-associated genes including CPT1A and ACADVL, and increases mitochondrial respiration. In some embodiments, FIGS. 12A-12C indicate that hormone-sensitive lipase promotes pro-resolving macrophage polarization and oxidative metabolism.

In some embodiments, increased hormone-sensitive lipase activity promotes a fatty acid oxidation program in a macrophage. In some embodiments, increased LIPE expression increases expression of one or more fatty acid oxidation-associated genes including CPT1A, ACADVL, or both. In some embodiments, increased hormone-sensitive lipase activity increases basal oxygen consumption rate, maximal oxygen consumption rate, spare respiratory capacity, ATP-linked respiration, or a combination thereof. In some embodiments, hormone-sensitive lipase-mediated lipid droplet lipolysis supports mitochondrial oxidative metabolism and thereby promotes efferocytosis and a pro-resolving macrophage phenotype.

In some embodiments, HSL activation includes phosphorylation at one or more activating residues including serine (Ser) at positions 563, 659, 660 (Ser563, Ser659, Ser660), or a combination thereof. In some embodiments, HSL activation further includes translocation or colocalization of HSL with lipid droplets. In some embodiments, HSL-mediated lipid droplet lipolysis provides fatty acids for mitochondrial oxidative metabolism. In some embodiments, increased HSL activity increases expression of fatty acid oxidation-associated genes including CPT1A, ACADVL, or both, and increases maximal oxygen consumption rate, spare respiratory capacity, ATP production, or combinations thereof.

As used herein, the term “phosphorylation of hormone-sensitive lipase at Ser660” refers to phosphorylation at serine residue 660 of hormone-sensitive lipase or the corresponding activating residue of a species homolog. As used herein, the term “translocation of hormone-sensitive lipase to lipid droplets” refers to increased localization, recruitment, colocalization, or association of hormone-sensitive lipase with one or more lipid droplets.

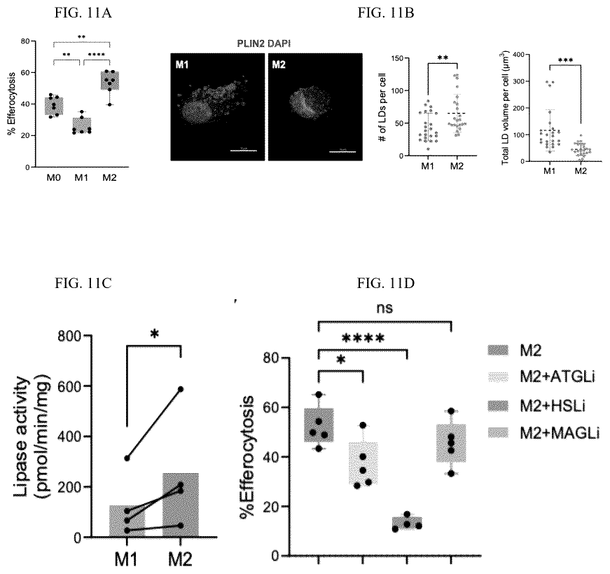

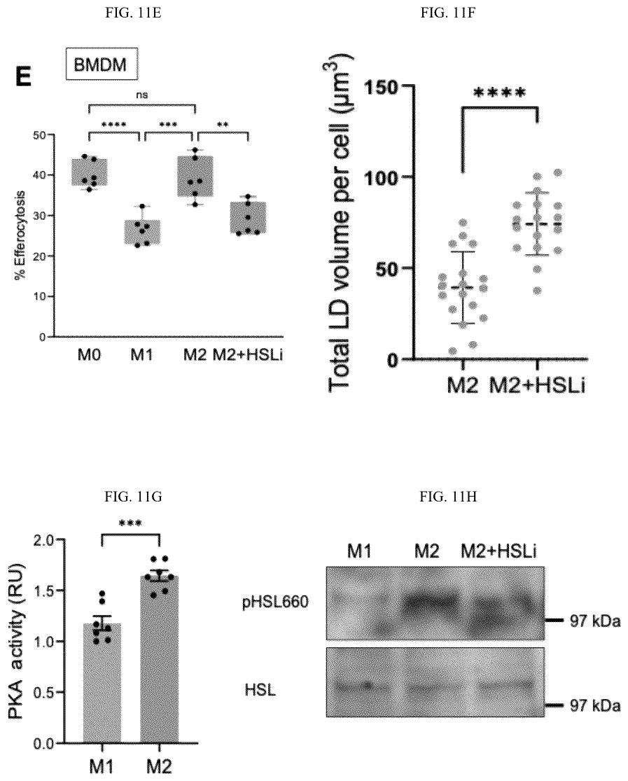

In some embodiments, FIGS. 11A-11H collectively show that efferocytosis is increased in M2 macrophages relative to M1 macrophages, that M1 macrophages accumulate larger and more numerous lipid droplets per cell, that lipase activity and protein kinase A activity are increased in M2 macrophages, and that inhibition of hormone-sensitive lipase decreases phosphorylation of hormone-sensitive lipase at Ser660, impairs efferocytosis in human and murine macrophages, and increases lipid droplet volume toward an M1-like state. In some embodiments, FIGS. 11A-11H therefore indicate that protein kinase A-mediated activation of hormone-sensitive lipase promotes lipid droplet remodeling and supports a pro-resolving macrophage phenotype.

In some embodiments, mechanistic specificity of hormone-sensitive lipase function is evaluated by genetic reduction or deletion of LIPE in a myeloid cell, a macrophage lineage cell, or a myeloid lineage animal model. In some embodiments, the genetic reduction includes siRNA, shRNA, antisense oligonucleotide, CRISPR-based repression, conditional deletion, or a combination thereof. In some embodiments, the conditional deletion is a myeloid-selective LIPE deletion. In some embodiments, reduced LIPE expression or activity decreases efferocytosis, decreases Annexin A1 production or secretion, decreases one or more M2-associated markers, increases inflammatory cytokine production, increases lipid droplet accumulation, or a combination thereof. In some embodiments, adipose triglyceride lipase also contributes to macrophage efferocytosis and lipid droplet remodeling. In some embodiments, reduced adipose triglyceride lipase activity decreases efferocytosis, and increased adipose triglyceride lipase expression or activity increases efferocytosis. In some embodiments, hormone-sensitive lipase and adipose triglyceride lipase function in a coordinated lipolytic pathway that supports mitochondrial oxidative metabolism and a pro-resolving macrophage phenotype.

### Membrane Remodeling and Efferocytosis Synapse Organization

In some embodiments, HSL-mediated lipid remodeling increases membrane fluidity and membrane plasticity. In some embodiments, such remodeling facilitates phagocytic cup formation, receptor clustering, receptor polarization, phosphoinositide-dependent signaling, phosphatidylinositol-3,4,5-trisphosphate recruitment, actin remodeling, or combinations thereof at an apoptotic cell contact site. In some embodiments, receptors participating in such organization include MERTK, T-cell immunoglobulin and mucin domain-containing protein 4 (TIM-4), CD36, CD206, RAGE, LAIR-1, or combinations thereof. In some embodiments, bridging molecules include growth arrest-specific gene 6 (Gas6), Protein S, or both. In some embodiments, macrophage function is assessed by one or more of efferocytosis, serial engulfment capacity, membrane fluidity, receptor organization, mitochondrial respiration, cytokine production, or combinations thereof.

As used herein, the term “membrane fluidity” refers to a physical state of a cellular membrane associated with membrane remodeling, lipid organization, receptor mobility, or phagocytic function. As used herein, the term “serial engulfment capacity” refers to the ability of a macrophage to engulf more than one apoptotic cell over time. As used herein, the term “receptor clustering” refers to increased spatial concentration, aggregation, enrichment, grouping, or local accumulation of one or more receptors in a defined region of a macrophage membrane, including at or near an efferocytosis synapse or an apoptotic cell contact site. In some embodiments, receptor clustering is determined by imaging, fluorescence intensity, puncta number, proximity analysis, colocalization analysis, synapse-associated enrichment, or another assay that detects localized receptor organization.

As used herein, the term “receptor polarization” refers to asymmetric redistribution, directional enrichment, or preferential localization of one or more receptors toward a particular region of a macrophage, including toward an apoptotic cell contact site, phagocytic cup, or efferocytosis synapse. In some embodiments, receptor polarization reflects organized receptor movement or retention associated with apoptotic cell recognition, binding, engulfment, or signaling.

As used herein, the term “apoptotic cell contact site” refers to a region of a macrophage membrane that contacts, binds, surrounds, engulfs, internalizes, or otherwise interfaces with an apoptotic cell during efferocytosis. In some embodiments, the apoptotic cell contact site includes an efferocytosis synapse, a phagocytic cup, a membrane-apposition region, or another localized interface at which receptor organization, phosphoinositide signaling, actin remodeling, or engulfment-associated signaling occurs.

In some embodiments, hormone-sensitive lipase-driven lipid droplet lipolysis supports remodeling of a macrophage plasma membrane during efferocytosis. In some embodiments, such remodeling increases membrane fluidity, membrane plasticity, receptor mobility, receptor clustering, receptor polarization, synapse formation, phagocytic cup formation, phosphoinositide-dependent signaling, actin remodeling, or a combination thereof at an apoptotic cell contact site. In some embodiments, the receptor organization includes organization of one or more efferocytosis-associated receptors selected from MERTK, TIM-4, CD36, CD206, RAGE, LAIR-1, or a combination thereof. In some embodiments, a macrophage exposed to HMGB1 in combination with C1q exhibits increased hormone-sensitive lipase-dependent membrane remodeling relative to a macrophage exposed to HMGB1 alone, thereby promoting a pro-resolving or M2-like macrophage phenotype. In some embodiments, impaired hormone-sensitive lipase activity in a macrophage from a subject having systemic lupus erythematosus is associated with reduced membrane fluidity, impaired receptor organization at an apoptotic cell contact site, impaired efferocytosis, or a combination thereof.

In some embodiments, membrane fluidity of a macrophage is measured using a fluorescence-based membrane fluidity assay. In some embodiments, an M2 macrophage or an M2-like macrophage exhibits greater membrane fluidity than an M1 macrophage or an M1-like macrophage, respectively. In some embodiments, inhibition of hormone-sensitive lipase decreases membrane fluidity in an M2 macrophage, an M2-like macrophage, or both, thereby indicating that hormone-sensitive lipase-dependent lipid remodeling contributes to membrane plasticity associated with a pro-resolving macrophage phenotype. In some embodiments, one or more scavenger receptors associated with a pro-resolving or M2 macrophage phenotype include CD36, CD206, or both. In some embodiments, increased hormone-sensitive lipase expression or activity increases surface expression of CD36, CD206, or both, and inhibition of hormone-sensitive lipase decreases such surface expression. In some embodiments, these findings indicate that hormone-sensitive lipase enzymatic activity contributes to establishment or maintenance of a surface phenotype associated with an M2 or M2-like macrophage.

In some embodiments, FIGS. 16A-16B show that M2 and M2-like macrophages exhibit greater plasma membrane fluidity and higher surface expression of CD206 and CD36 than corresponding M1 and M1-like macrophages, and that inhibition of hormone-sensitive lipase decreases membrane fluidity and reduces surface expression of one or more scavenger receptors. In some embodiments, FIGS. 16A-16B indicate that hormone-sensitive lipase-dependent lipid remodeling supports membrane properties and surface receptor organization associated with efferocytosis.

In some embodiments, lipid remodeling associated with hormone-sensitive lipase activity is assessed in a plasma membrane fraction of a macrophage. In some embodiments, the plasma membrane fraction is analyzed by lipidomics, including untargeted lipidomics, to identify one or more lipid species associated with membrane fluidity, receptor mobility, phagocytic cup formation, or efferocytosis. In some embodiments, organization of one or more efferocytosis-associated receptors at an apoptotic cell contact site is measured by determining receptor clustering, receptor polarization, receptor colocalization, synapse formation, or a combination thereof over time. In some embodiments, such receptors include MERTK, TIM-4, CD36, CD206, RAGE, LAIR-1, or a combination thereof.

In some embodiments, lipid species associated with a pro-resolving macrophage phenotype include one or more phosphatidylinositol species, one or more lysophosphatidylinositol species, nervonic acid, or a combination thereof. In some embodiments, a phosphatidylinositol species includes PI(18:0_18:0). In some embodiments, such lipid species are associated with increased membrane organization, increased phosphoinositide signaling, increased receptor mobility, increased mitochondrial fitness, increased efferocytosis, or a combination thereof. In some embodiments, lipidomic analysis is used to identify a plasma membrane lipid signature or a lipid droplet-associated lipid signature associated with a pro-resolving macrophage phenotype. In some embodiments, a macrophage exposed to HMGB1 in combination with C1q exhibits a lipid signature associated with a pro-resolving or M2-like state relative to a macrophage exposed to HMGB1 alone.

### Screening Assays for Pro-Resolving Macrophage Activity

In some embodiments, an agent or a candidate agent is screened for the ability to promote a pro-resolving macrophage activity by contacting a macrophage with the agent and measuring one or more functional or mechanistic readouts associated with enhanced efferocytosis. In some embodiments, the term candidate agent refers to an agent evaluated for the ability to alter one or more macrophage functions associated with a pro-resolving phenotype. In some embodiments, the candidate agent includes a small molecule, lipid, fatty acid, phospholipid, lysophospholipid, peptide, polypeptide, protein, antibody, nucleic acid, gene-modulating construct, agonist, antagonist, inhibitor, activator, cytokine, chemokine, complement component, damage-associated molecular pattern, neurotransmitter, hormone, metabolite, extract, particle, nanoparticle, cell-derived product, or a combination thereof. In some embodiments, the candidate agent is screened for the ability to increase efferocytosis, increase lipid droplet lipolysis, alter lipid droplet composition, increase mitochondrial respiration, increase membrane fluidity, promote receptor organization at an efferocytosis synapse, reduce inflammatory signaling, promote a pro-resolving macrophage phenotype, or a combination thereof. As used herein, the term “agent” refers to any compound, molecule, composition, formulation, mixture, biological material, stimulus, condition, or treatment capable of being contacted with a cell, tissue, sample, or subject and evaluated for a biological effect. In some embodiments, an agent includes a small molecule, lipid, fatty acid, phospholipid, lysophospholipid, peptide, polypeptide, protein, antibody, nucleic acid, gene-modulating construct, agonist, antagonist, inhibitor, activator, cytokine, chemokine, complement component, damage-associated molecular pattern, neurotransmitter, hormone, metabolite, extract, particle, nanoparticle, cell-derived product, or a combination thereof.

As used herein, the term “pro-resolving macrophage activity” refers to one or more measurable macrophage activities associated with a pro-resolving macrophage state or phenotype. In some embodiments, pro-resolving macrophage activity includes increased efferocytosis, increased serial engulfment capacity, increased lipid droplet remodeling, increased lipid droplet lipolysis, increased mitochondrial respiration, increased oxidative metabolism, increased membrane fluidity, increased receptor organization at an efferocytosis synapse, increased expression or activity of one or more pro-resolving markers, reduced inflammatory mediator production, or a combination thereof. In some embodiments, increased pro-resolving macrophage activity is determined relative to a suitable reference, including a control macrophage, an untreated macrophage, a vehicle-treated macrophage, an inflammatory macrophage, or a baseline measurement from the same macrophage population.

As used herein, the term “control macrophage” refers to a macrophage used as a comparator in an assay. In some embodiments, the control macrophage is of the same source, lineage, preparation, donor, polarization condition, genotype, disease state, or differentiation protocol as a test macrophage, except that the control macrophage is not contacted with a candidate agent or is contacted with a vehicle, carrier, negative control, comparator treatment, or reference treatment. In some embodiments, the control macrophage provides a reference for determining whether contact with a candidate agent alters efferocytosis, membrane properties, receptor organization, lipid metabolism, inflammatory signaling, or another disclosed macrophage function.

As used herein, the term “candidate agent” refers to an agent evaluated for the ability to alter one or more macrophage functions associated with a pro-resolving phenotype or activity. In some embodiments, the candidate agent includes a small molecule, lipid, fatty acid, phospholipid, lysophospholipid, peptide, polypeptide, protein, antibody, nucleic acid, gene-modulating construct, agonist, antagonist, inhibitor, activator, cytokine, chemokine, complement component, damage-associated molecular pattern, neurotransmitter, hormone, metabolite, extract, particle, nanoparticle, cell-derived product, formulation, or a combination thereof. In some embodiments, a candidate agent is selected based on its ability to increase efferocytosis, increase hormone-sensitive lipase activation, increase membrane fluidity, promote receptor organization at an efferocytosis synapse, promote a pro-resolving macrophage activity, or a combination thereof.

In some embodiments, the readout includes hormone-sensitive lipase phosphorylation, including phosphorylation at Ser660, translocation of hormone-sensitive lipase to lipid droplets, efferocytosis, membrane fluidity, receptor clustering at an apoptotic cell contact site, serial engulfment capacity, mitochondrial respiration, expression of PPARγ, expression of CD36, expression of CD163, expression of CD206, reduction in inflammatory cytokine production, or a combination thereof. In some embodiments, the agent is selected when one or more of such readouts is increased relative to a control macrophage not contacted with the agent. In some embodiments, the macrophage is derived from a healthy subject, a subject having systemic lupus erythematosus, a subject having lupus nephritis, or an in vitro polarized macrophage population. In some embodiments, screening identifies an agent that promotes a pro-resolving lipid signature, increases macrophage metabolic fitness, reduces lipid droplet accumulation, or enhances apoptotic cell clearance.

In some embodiments, screening further includes assessing whether the agent increases membrane fluidity, alters plasma membrane lipid composition, promotes receptor clustering kinetics at an efferocytosis synapse, or a combination thereof. In some embodiments, the macrophage screened is derived from a subject having systemic lupus erythematosus, and an agent is selected when the agent restores one or more defective functions relative to an untreated SLE-derived macrophage.

In some embodiments, the disclosure provides a method of screening ex vivo for a candidate agent that promotes pro-resolving macrophage activity, including: incubating a macrophage with a candidate agent in the presence of high mobility group box 1; contacting the macrophage with an apoptotic cell; measuring efferocytosis of the macrophage relative to a control macrophage incubated in the presence of high mobility group box 1 and a vehicle; measuring, relative to the control macrophage, at least one parameter selected from the group consisting of: phosphorylation of hormone-sensitive lipase at Ser660, membrane fluidity, and receptor organization at an apoptotic cell contact site, where the receptor organization includes receptor clustering or receptor polarization of one or more receptors selected from the group consisting of MERTK, TIM-4, CD36, CD206, receptor for advanced glycation end products, leukocyte-associated immunoglobulin-like receptor-1, and combinations thereof; and identifying the candidate agent as promoting pro-resolving macrophage activity when the macrophage exhibits increased efferocytosis and an increase in the at least one parameter relative to the control macrophage.

Disease Models, Subject-Derived Cells, and In Vivo Evaluation

In some embodiments, the disclosed methods are performed using monocytes or macrophages from subjects having SLE. In some embodiments, the cells include CD14+CD16− monocytes, CD14+CD16+ monocytes, or both. In some embodiments, such subject-derived cells exhibit reduced HSL activation, reduced ASAH1 activity, reduced nervonic acid abundance, reduced membrane fluidity, reduced mitochondrial respiration, increased lipid droplet accumulation, impaired efferocytosis, or combinations thereof relative to corresponding cells from healthy subjects. In some embodiments, such defects are assessed before and after treatment with a lipase activator, a PDE3 inhibitor, an adrenergic agonist, nervonic acid, C1q, or combinations thereof.

In some embodiments, macrophages or monocytes obtained from different human donors exhibit different magnitudes of response to HMGB1, C1q, or a combination thereof with respect to lipid droplet remodeling, mitochondrial respiration, cytokine production, protein kinase A activity, hormone-sensitive lipase activation, or efferocytosis. In some embodiments, such donor-to-donor variation is addressed by evaluating cells from multiple donors, increasing the number of biological replicates, repeating one or more assays across independent donor sets, or a combination thereof. In some embodiments, mitochondrial respiration is measured by extracellular flux analysis, high-resolution respirometry, or both. In some embodiments, high-resolution respirometry includes Oroboros respirometry. In some embodiments, mitochondrial function is assessed by one or more of basal respiration, maximal respiration, spare respiratory capacity, ATP-linked respiration, oxygen consumption rate, extracellular acidification rate, oxygen consumption to extracellular acidification ratio, mitochondrial membrane potential, mitochondrial morphology, mitochondrial reactive oxygen species, or a combination thereof.

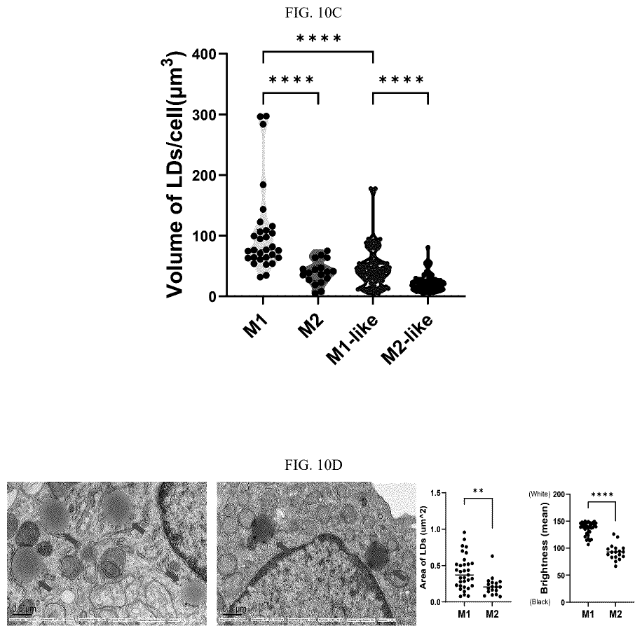

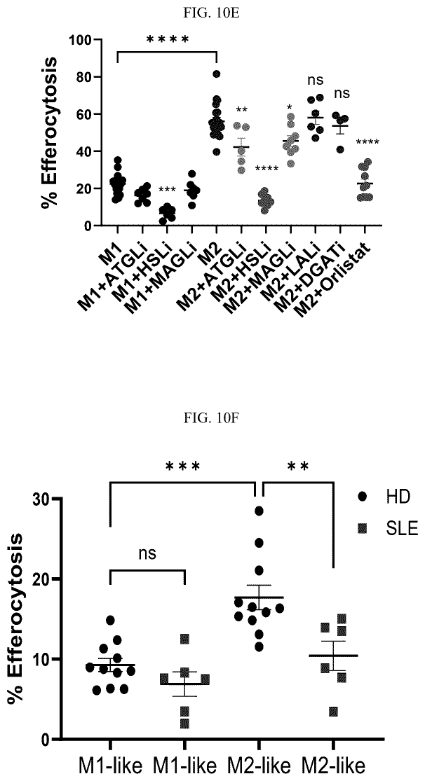

In some embodiments, FIGS. 10A-10F demonstrate that macrophage subsets differ in lipid droplet architecture, lipolytic dependence, and efferocytic function, and that such differences are relevant to inflammatory and autoimmune settings including systemic lupus erythematosus (SLE). In some embodiments, the differentiation scheme shown in FIG. 10A and the lipid droplet imaging and quantification shown in FIGS. 10B-10D indicate that M1, M1-like, M2, and M2-like macrophages differ in lipid droplet size, volume, density, dispersal, ultrastructure, or a combination thereof. In some embodiments, M2 or M2-like macrophages are associated with a lipid droplet state that is more dispersed, more readily remodeled, or otherwise more compatible with efferocytic activity than that of M1 or M1-like macrophages. In some embodiments, the efferocytosis data shown in FIG. 10E indicate that lipase activity contributes to macrophage clearance of apoptotic cells and that inhibition of hormone-sensitive lipase or another lipase reduces efferocytic activity. In some embodiments, the efferocytosis data shown in FIG. 10F indicate that monocyte-derived macrophages from healthy donors and subjects having SLE respond differently to HMGB1 or HMGB1 plus C1q treatment, thereby demonstrating that inflammatory context and disease state influence macrophage lipid remodeling and efferocytosis. In some embodiments, FIGS. 10A-10F collectively support that modulation of lipid droplet composition, lipid droplet dispersal, or lipid droplet lipolysis is used to promote a pro-resolving or M2-like macrophage phenotype, enhance efferocytosis, and improve macrophage function in a subject having SLE or another inflammatory condition.

In some embodiments, lipid droplets in M1, M1-like, M2, and M2-like macrophages differ with respect to size, abundance, composition, subcellular distribution, density, dispersal, or a combination thereof. In some embodiments, lipid droplets associated with an M2 or M2-like macrophage state are more readily remodeled or hydrolyzed than lipid droplets associated with an M1 or M1-like macrophage state. In some embodiments, breakdown of lipid droplet components releases one or more fatty acids or related lipid species that enhance efferocytosis in response to apoptotic cells. In some embodiments, manipulation of lipid droplet content, lipid droplet composition, lipid droplet dispersal, or lipolysis alters macrophage functionality, including macrophage polarization, efferocytosis, mitochondrial metabolism, inflammatory mediator production, membrane remodeling, or a combination thereof.

In some embodiments, a macrophage, monocyte-derived macrophage, or monocyte obtained from a subject having systemic lupus erythematosus responds differently to apoptotic cells than a corresponding cell obtained from a healthy control. In some embodiments, the different response includes reduced efferocytosis, altered lipid droplet accumulation, altered lipid droplet composition, reduced lipase activity, reduced hormone-sensitive lipase activation, impaired metabolic remodeling, or a combination thereof. In some embodiments, a disclosed composition or method is used to alter lipid droplet content or lipolytic activity in a macrophage from a subject having systemic lupus erythematosus, thereby improving macrophage functionality and promoting a more pro-resolving phenotype.

In some embodiments, impaired macrophage efferocytosis in systemic lupus erythematosus is associated with defective adrenergic responsiveness, defective protein kinase A activation, defective hormone-sensitive lipase activation, impaired lipid droplet remodeling, or a combination thereof. In some embodiments, a macrophage obtained from a subject having systemic lupus erythematosus or lupus nephritis is contacted ex vivo with norepinephrine, a beta-adrenergic receptor agonist, a beta2-adrenergic receptor agonist, or a combination thereof for a time sufficient to increase one or more pro-resolving macrophage activities. In some embodiments, the pro-resolving macrophage activities include increased efferocytosis, increased serial engulfment capacity, increased phosphorylation of hormone-sensitive lipase at Ser660, increased localization of hormone-sensitive lipase to a lipid droplet, reduced lipid droplet burden, increased mitochondrial respiration, increased PPARγ expression, or a combination thereof. In some embodiments, the macrophage before or after adrenergic stimulation is evaluated to determine whether adrenergic stimulation restores a pro-resolving efferocytosis program in a lupus-associated macrophage population.

In some embodiments, a macrophage obtained from a subject having systemic lupus erythematosus is contacted ex vivo or in vivo with a pharmacologic activator of hormone-sensitive lipase or with an agent that increases hormone-sensitive lipase activation through a protein kinase A-dependent pathway. In some embodiments, the treatment increases lipid droplet lipolysis, improves plasma membrane remodeling, increases membrane fluidity, promotes receptor organization at an apoptotic cell contact site, increases mitochondrial oxidative metabolism, increases efferocytosis, or a combination thereof. In some embodiments, the treatment restores one or more defective pro-resolving macrophage functions in a macrophage population obtained from a subject having systemic lupus erythematosus or lupus nephritis.

In some embodiments, the pharmacologic activator of hormone-sensitive lipase includes an adrenergic agonist, a beta-adrenergic receptor agonist, a beta2-adrenergic receptor agonist, an agent that increases cyclic adenosine 3′,5′-monophosphate signaling, an agent that decreases phosphodiesterase 3A activity or expression, or a combination thereof. In some embodiments, the activator is evaluated for the ability to restore membrane fluidity, plasma membrane remodeling, receptor clustering at an apoptotic cell contact site, receptor polarization, mitochondrial oxidative metabolism, serial engulfment capacity, or a combination thereof in a macrophage obtained from a subject having systemic lupus erythematosus. In some embodiments, restoration of one or more such functions indicates reversal of an intrinsic or acquired hormone-sensitive lipase-associated macrophage dysfunction.

In some embodiments, in vivo pathway activity is evaluated in an HMGB1-induced sterile peritonitis model, a C1q-deficient model, a lupus-prone model, or combinations thereof. In some embodiments, in vivo efferocytosis is quantified in peritoneal myeloid cells after administration of apoptotic thymocytes. In some embodiments, peritoneal or tissue macrophages are evaluated for pHSL, PKA-pCREB signaling, PLIN2, ASAH1, PPARγ, CD36, CD206, MERTK, or combinations thereof. In some embodiments, kidney tissue from a lupus-prone subject or animal is further evaluated by spatial tissue analysis or spatial proteomics to map macrophage populations expressing one or more of ASAH1, PLIN2, PPARγ, CD68, CD14, CD206, MERTK, and CD36.

In some embodiments, in vivo analysis of HMGB1-and C1q-regulated macrophage responses is performed at one or more time points selected from about 6 hours, about 12 hours, about 24 hours, or later after administration of an inflammatory stimulus, C1q, or both. In some embodiments, when a model exhibits altered recruitment, altered retention, or altered recovery of peritoneal macrophages or myeloid cells, pathway activity and macrophage subset composition are evaluated at an earlier time point, a later time point, or both. In some embodiments, peritoneal myeloid cells are characterized by one or more of abundance, subset distribution, protein kinase A pathway activity, phosphorylated hormone-sensitive lipase, phosphorylated cAMP response element-binding protein, phosphorylated AMP-activated protein kinase, lipid droplet burden, cytokine expression, Arg1 expression, IL-10 expression, or efferocytosis. In some embodiments, such temporal analysis is used to distinguish a defect in macrophage recruitment from a defect in macrophage polarization, lipid remodeling, metabolic activation, or efferocytic function.

In some embodiments, intraperitoneal administration of HMGB1 produces an inflammatory milieu characterized by increased leukotriene B4, increased TNF expression, increased IRF5 expression, or a combination thereof in a peritoneal exudate, a peritoneal myeloid cell population, or both. In some embodiments, co-administration of C1q with HMGB1 shifts the inflammatory milieu toward a pro-resolving state characterized by decreased leukotriene B4, increased resolvin D, increased IL-10 expression, increased Arg1 expression, or a combination thereof. In some embodiments, such in vivo changes indicate that C1q counteracts HMGB1-associated inflammatory programming and promotes a pro-resolving or M2-like macrophage phenotype. In some embodiments, the sterile peritonitis model is used to evaluate an agent that increases cAMP signaling, decreases phosphodiesterase 3A activity or expression, increases protein kinase A activity, increases hormone-sensitive lipase activity, promotes lipid droplet remodeling, enhances efferocytosis, or a combination thereof.

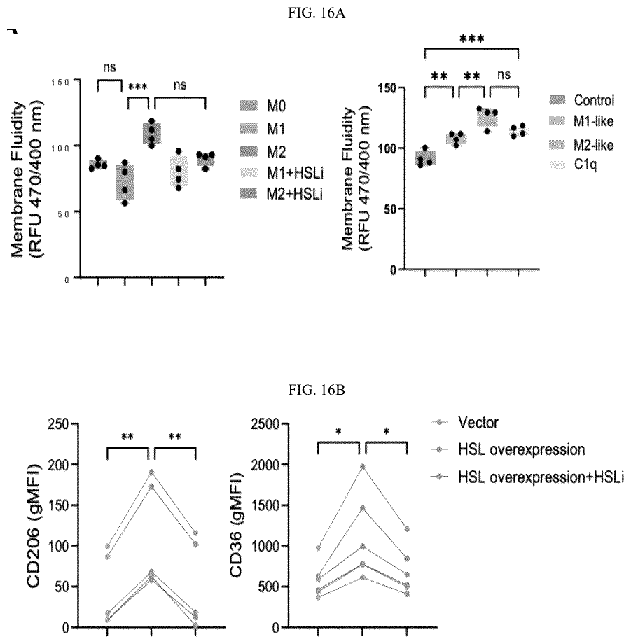

In some embodiments, FIGS. 15A-15C show that co-administration of HMGB1 and C1q in a sterile peritonitis model decreases leukotriene B4, increases resolvin D, and shifts macrophage-associated gene expression toward a pro-resolving or M2-like profile. In some embodiments, FIGS. 15A-15C indicate that C1q counteracts HMGB1-associated inflammatory programming in vivo and promotes a pro-resolving macrophage state.

In some embodiments, the macrophage evaluated in vivo or ex vivo includes a peritoneal macrophage, including a resident peritoneal macrophage, an elicited peritoneal macrophage, or a peritoneal macrophage recovered after induction of sterile peritonitis. In some embodiments, peritoneal macrophages are assessed for lipid droplet burden, hormone-sensitive lipase activation, protein kinase A pathway activity, mitochondrial function, membrane remodeling, receptor organization, efferocytosis, or a combination thereof.

In some embodiments, neuroimmune stimulation is used in vivo to enhance norepinephrine-dependent activation of a beta-adrenergic receptor-protein kinase A-hormone-sensitive lipase pathway in a lupus-prone subject or animal model. In some embodiments, the neuroimmune stimulation includes vagus nerve stimulation. In some embodiments, vagus nerve stimulation, alone or in combination with administration of norepinephrine or a beta-adrenergic receptor agonist, increases macrophage efferocytosis, increases hormone-sensitive lipase activation, promotes lipid droplet remodeling, increases mitochondrial oxidative metabolism, or a combination thereof in vivo. In some embodiments, the subject or animal model includes a subject having systemic lupus erythematosus, a subject having lupus nephritis, an SLETC mouse, an NZB/W F1 mouse, or a combination thereof. In some embodiments, treatment response is assessed by measuring macrophage efferocytosis, hormone-sensitive lipase phosphorylation, protein kinase A pathway activity, lipid droplet burden, renal inflammation, proteinuria, renal histopathology, or a combination thereof. In some embodiments, kidney tissue, spleen tissue, lymphoid tissue, inflamed tissue, or a combination thereof is evaluated by spatial tissue analysis to identify macrophage populations associated with adrenergic pathway activation, hormone-sensitive lipase activation, lipid droplet remodeling, or restored efferocytosis. In some embodiments, spatial analysis includes detection of one or more of hormone-sensitive lipase, phosphorylated hormone-sensitive lipase, PLIN2, PPARγ, CD68, CD14, CD206, MERTK, CD36, or combinations thereof.

In some embodiments, a method of treating a disease or condition associated with impaired macrophage efferocytosis, inflammatory macrophage polarization, dysregulated lipid metabolism, or a combination thereof includes administering vagus nerve stimulation to a subject in need thereof, optionally in combination with administration of norepinephrine, a beta-adrenergic receptor agonist, a beta2-adrenergic receptor agonist, or a combination thereof, thereby increasing cyclic adenosine 3′,5′-monophosphate signaling, protein kinase A activity, hormone-sensitive lipase activation, lipid droplet remodeling, mitochondrial oxidative metabolism, membrane remodeling, efferocytosis, or combinations thereof. In some embodiments, the disease or condition includes systemic lupus erythematosus, lupus nephritis, chronic inflammation, autoimmunity, a neuroinflammatory disease, or a neurodegenerative disease. In some embodiments, treatment response is assessed by measuring one or more of macrophage efferocytosis, hormone-sensitive lipase phosphorylation, protein kinase A pathway activity, lipid droplet burden, renal inflammation, proteinuria, renal histopathology, or combinations thereof.

In some embodiments, kidney tissue is evaluated to identify a macrophage population associated with lipid droplet remodeling, efferocytosis, or both. In some embodiments, the kidney tissue is obtained from a subject having lupus nephritis or from a lupus-prone animal model. In some embodiments, the kidney tissue is evaluated by spatial tissue analysis, immunohistochemistry, immunofluorescence, spatial proteomics, spatial transcriptomics, imaging mass spectrometry, or a combination thereof. In some embodiments, the evaluation includes spatially detecting expression of one or more of ASAH1, PLIN2, PPARγ, MERTK, CD36, CD68, CD14, or CD206. In some embodiments, co-localization or regional enrichment of such markers identifies a macrophage population associated with a pro-resolving phenotype, an inflammatory phenotype, altered lipid droplet burden, altered efferocytosis, altered ceramide metabolism, altered nervonic acid abundance, or a combination thereof. In some embodiments, the spatially detected marker pattern is used to assess disease state, monitor treatment response, identify a target tissue macrophage population, or evaluate the effect of treatment with a lipase modulator, nervonic acid, C1q, a PDE3 inhibitor, or a combination thereof. As used herein, the term “kidney tissue” refers to renal tissue, including tissue obtained from a subject having lupus nephritis in some embodiments. As used herein, the term “spatially detecting” refers to detecting a marker in a manner that preserves positional information within tissue.

In some embodiments, kidney tissue from a subject having lupus nephritis, or from a lupus-prone animal model, is evaluated before, during, or after treatment to identify a macrophage population associated with lipid droplet remodeling, altered efferocytosis, altered ceramide metabolism, or a combination thereof. In some embodiments, the kidney tissue is evaluated by spatial tissue analysis, immunohistochemistry, immunofluorescence, spatial proteomics, spatial transcriptomics, imaging mass spectrometry, or a combination thereof. In some embodiments, the evaluation includes spatially detecting one or more of N-acylsphingosine amidohydrolase 1, PLIN2, PPARγ, MERTK, CD36, CD68, CD14, CD206, hormone-sensitive lipase, phosphorylated hormone-sensitive lipase, or combinations thereof. In some embodiments, the spatially detected marker pattern is used to assess disease state, identify a target tissue macrophage population, monitor treatment response, or evaluate the effect of treatment with a lipase modulator, nervonic acid, C1q, a phosphodiesterase 3 inhibitor, a beta-adrenergic receptor agonist, neuroimmune stimulation, or a combination thereof.

### EXAMPLES

The following examples illustrate specific aspects of the instant description. The examples should not be construed as limiting, as the example merely provides specific understanding and practice of the embodiments and its various aspects.

### Example 1: Human M1 and M2 Macrophage Polarization and Lipid Droplet Characteristics

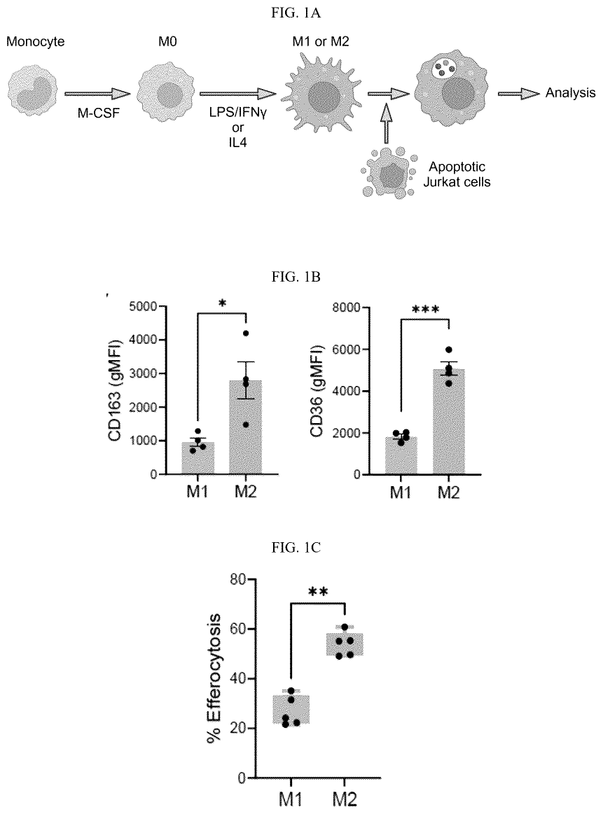

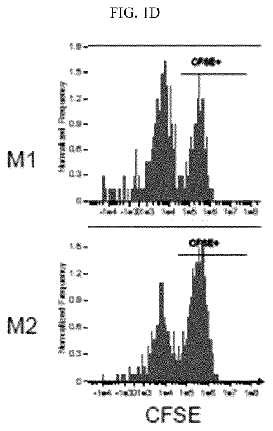

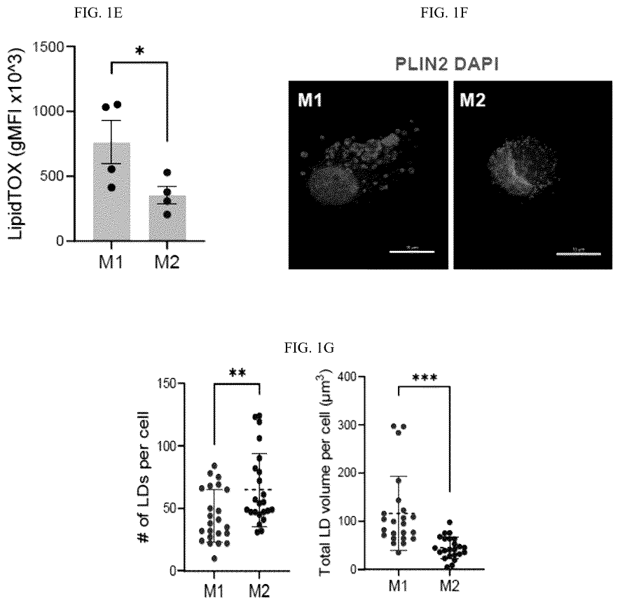

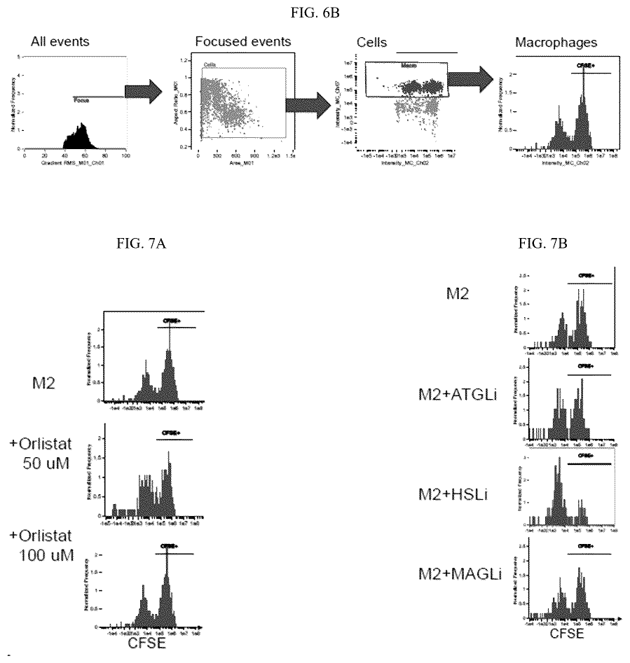

In order to determine whether lipid droplet (LD) characteristics significantly influence macrophage function, the efferocytosis capabilities of M1 and M2 macrophages differentiated from human blood monocytes were analyzed. Human blood monocytes from healthy donors were differentiated into monocyte-derived macrophages and polarized with IFN-γ and LPS to generate M1 macrophages or with IL-4 to generate M2 macrophages, as shown in FIG. 1A. CD14+monocytes were polarized into M1 macrophages using IFN-γ/LPS stimulation and into M2 macrophages using IL-4 treatment (FIG. 1A). Gene expression analysis confirmed polarization, with M1 macrophages exhibiting increased IL-1β and HIF-1α, while M2 macrophages showed elevated CD206, MERTK and PPARγ expression (FIG. 6A). The efferocytosis assay gating strategy is shown in FIG. 6B. In some embodiments, CytoTell™ Blue-positive macrophages were first identified, and CFSE positivity was then determined using single-stained controls to quantify engulfment of apoptotic cells. Enhanced surface expression of CD36 and CD163 on M2 macrophages further verified their anti-inflammatory phenotype (FIG. 1B). M2 macrophages demonstrated superior efferocytosis compared to M1 macrophages (FIGS. 1C-1D). To further test that LD features contributed to macrophage function, LD characteristics in polarized macrophages were analyzed. LipidTOX staining indicated a higher neutral lipid content in M1 than M2 macrophages (FIG. 1E). Furthermore, staining with PLIN2, which localizes to the periphery of LDs, and confocal microscopy showed higher total LD volume in M1 macrophages and smaller, more numerous LD in M2 macrophages, suggesting distinct metabolic states between the two phenotypes (FIG. 1F-1G). Lipid droplet number per cell and total lipid droplet volume per cell were analyzed, and M2 macrophages exhibited a greater number of lipid droplets but a lower total lipid droplet volume than M1 macrophages. PLIN2-based confocal imaging further supported that lipid droplet organization differed between M1 and M2 macrophages. These findings suggested that LD composition and organization contributed to functional differences in macrophage polarization, particularly in efferocytosis efficiency.

### Example 2: Hormone-Sensitive Lipase (HSL) Activity Is Critical For Efferocytosis in M2 Macrophages

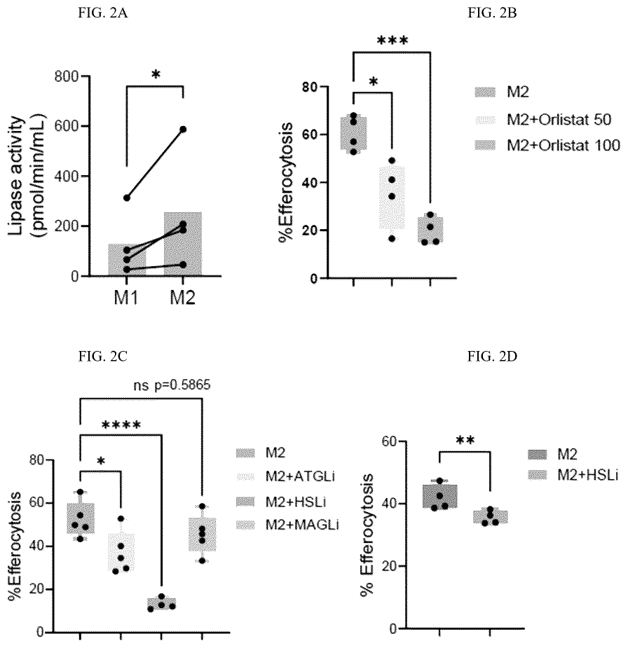

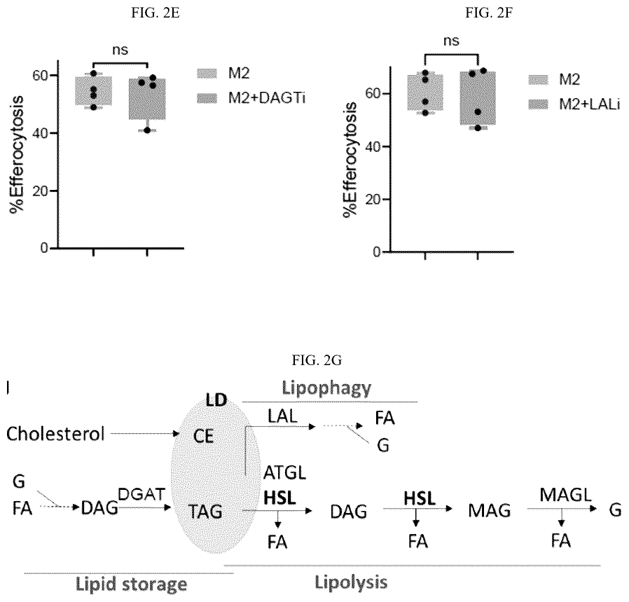

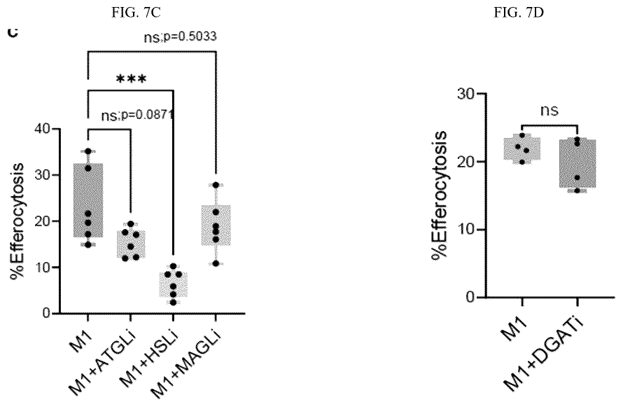

M2 macrophages exhibited higher lipase activity than M1 macrophages (FIG. 2A), indicating increased lipolysis in M2 cells. To assess the role of lipid degradation in efferocytosis, macrophages were treated with Orlistat, a pan-lipase inhibitor. To further investigate the contribution of lipolysis to efferocytosis in M2 macrophages, the cells were also treated with Atglistatin, HSL-IN-1, MJN110, or Lalistat1 and then evaluated in efferocytosis assays. A dose-dependent suppression of efferocytosis was observed (FIG. 2B, FIG. 7A). Subsequently, the impact of specific lipase inhibitors on efferocytosis was assessed by employing Atglistatin (an ATGL inhibitor), HSL-IN-1 (an HSL inhibitor), and MJN110 (a MAGL inhibitor). Inhibition of specific lipases, including ATGL and HSL, reduced efferocytosis of M2 macrophages and HSL inhibition also reduced efferocytosis of M1 macrophages (FIG. 2C, FIG. 7C). HSL inhibition in mouse bone marrow-derived M2 macrophages similarly reduced their efferocytosis (FIG. 2D). However, MAGL inhibition exhibited a negligible effect on the efferocytosis of both M2 and M1 macrophages (FIG. 2C, FIG. 7C). Inhibition of DGAT1 with T863 did not alter efferocytosis in either M1 or M2 macrophages (FIG. 2E, FIG. 7D). Despite reports indicating the critical role of lysosomal lipase in M2 activation, lysosomal lipase inhibition with Lalistat1 did not demonstrate any noticeable effect (FIG. 2F). Inhibition of ATGL or HSL reduced efferocytosis in M2 macrophages, and pan-lipase inhibition with Orlistat also suppressed efferocytosis. In contrast, inhibition of lysosomal lipase or DGAT1 did not significantly alter efferocytosis in M2 macrophages. These findings indicated that lipase-dependent lipid droplet remodeling contributed to the enhanced efferocytic activity of M2 macrophages, and that blockade of lipolysis impairsed that activity. These results suggested a critical role of HSL in regulating efferocytosis (FIG. 2G).

### Example 3: HSL Activation via PKA-dependent Phosphorylation and Macrophage Function

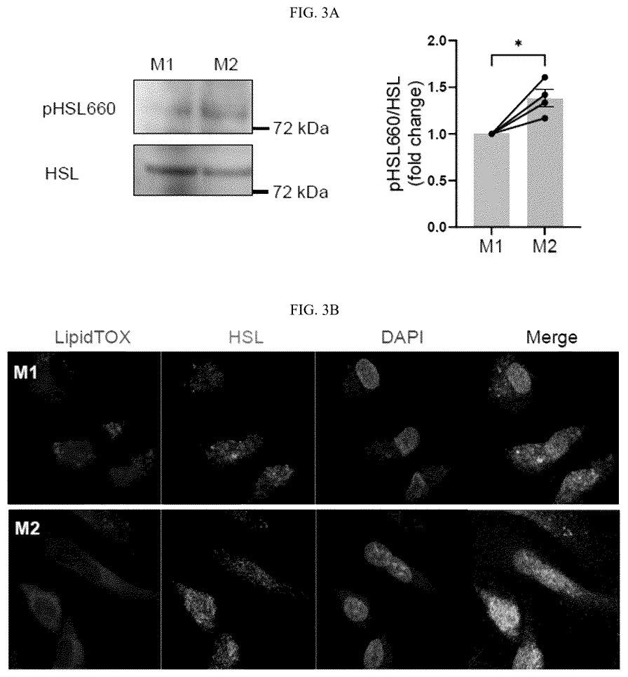

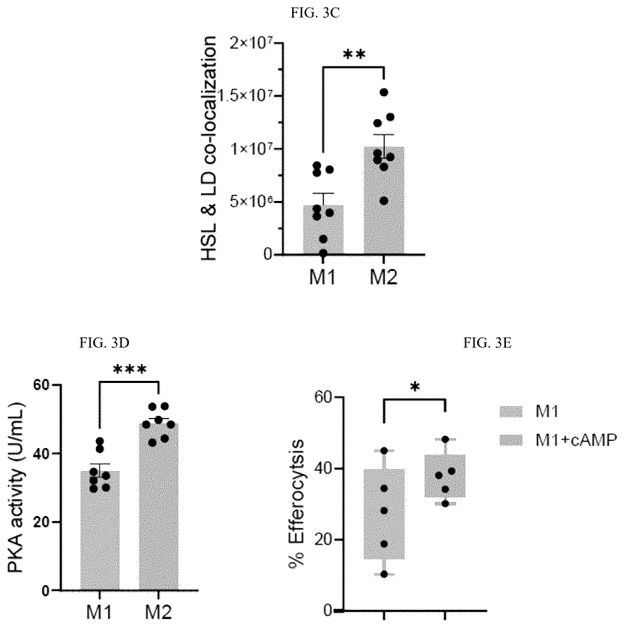

In M2 macrophages, HSL phosphorylation was elevated at Ser660, a modification that enhanced HSL activity (FIG. 3A). Confocal imaging showed HSL colocalization with LD in M2 macrophages (FIGS. 3B-3C), suggesting a role in LD metabolism. The cAMP-PKA signaling pathway, which is known to regulate M2 polarization, phosphorylated HSL at Ser660, thereby enhancing its activity. Accordingly, PKA activity was measured and found to be statistically significantly higher in M2 macrophages compared to M1 macrophages (FIG. 3D). To further test the role of PKA, M1 macrophages were treated with dibutyryl-cAMP, a PKA activator, which enhanced efferocytosis (FIG. 3E). These findings confirmed the role of PKA in promoting LD lipolysis through HSL activation, linking this pathway to enhanced efferocytic function in M2 macrophages.

### Example 4: HSL Inhibition Alters LD Features and Phenotypes in M2 Macrophages

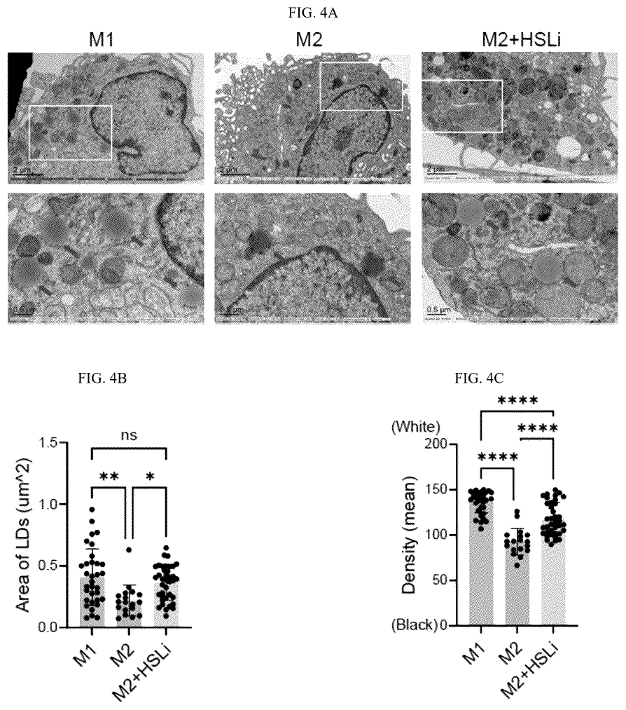

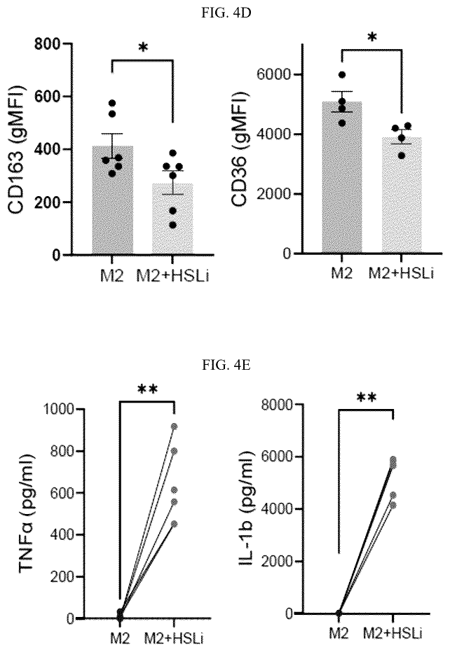

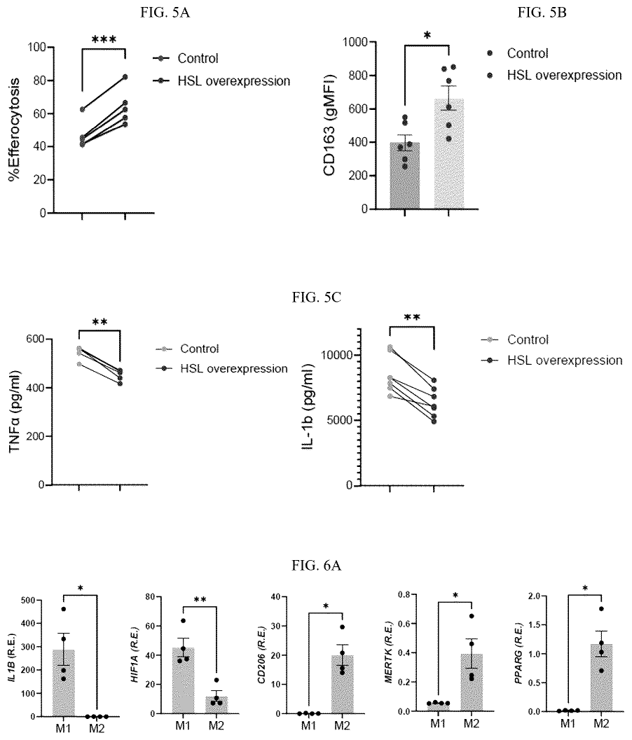

Transmission electron microscopy further showed morphological differences in lipid droplets between M1 and M2 macrophages, with M2 macrophages exhibiting smaller and denser lipid droplets than M1 macrophages. Electron microscopy further demonstrated that M2 macrophages exhibited smaller LD than M1 macrophages, and HSL inhibition increased LD size, shifting their morphology toward an M1-like profile (FIGS. 4A-4B). Analysis of LD density further showed that HSL inhibition altered LD condensation, making them less dense (whiter), resembling M1 macrophages (FIG. 4C). HSL inhibition also reduced M2 markers (CD163 and CD36) on the macrophage surface (FIG. 4D) and increased TNFα and IL-1β production (FIG. 4E), indicating a shift towards a more inflammatory state. Furthermore, HSL overexpression significantly enhanced efferocytosis and altered CD163 expression favoring the M2 phenotype (FIGS. 5A-5B). HSL overexpression also suppressed TNFα and IL-1β production (FIG. 5C). These results suggested that lipase activity, particularly HSL, is critical in regulating LD features and M2 macrophage polarization. These results further showed that efferocytosis was greater in M2 macrophages, whereas M1 macrophages accumulated larger and more numerous lipid droplets per cell. Lipase activity and protein kinase A activity were increased in M2 macrophages relative to M1 macrophages. Pharmacologic inhibition of hormone-sensitive lipase reduced phosphorylation of hormone-sensitive lipase at Ser660, impaired efferocytosis in both human macrophages and murine bone marrow-derived macrophages, and increased lipid droplet volume toward an M1-like state, as shown in FIGS. 11A-11H.

Additional experiments showed that siRNA-mediated reduction of LIPE decreased efferocytosis and Annexin A1 secretion and increased TNFα production in human macrophages. In complementary studies, increased LIPE expression increased efferocytosis and Annexin A1 secretion, reduced TNFα and IL-1β, increased expression of PPARγ, CD36, and CD200R1, and increased expression of CPT1A and ACADVL together with mitochondrial respiration, as shown in FIGS. 12A-12C.

### Example 5: HMGB1 and C1q Regulate Lipid Droplet Characteristics and Efferocytosis in Macrophages From Healthy Donors and Subjects Having Systemic Lupus Erythematosus

To evaluate the role of lipid droplets (LDs) in macrophage polarization in an inflammatory microenvironment relevant to systemic lupus erythematosus (SLE), macrophages were generated under conditions that produced M1-like and M2-like phenotypes. As shown schematically in FIG. 10A, monocyte-derived macrophages from healthy donors or subjects having SLE were differentiated and then exposed to HMGB1 to generate M1-like macrophages or to HMGB1 plus C1q to generate M2-like macrophages. For selected experiments, macrophages were cultured in 24-well plates in X-VIVO 15 serum-free medium at about 4×10⁵ to 5×10⁵ cells per well. LD characteristics were assessed by staining with anti-perilipin 2 (PLIN2) antibody and confocal imaging. As shown in FIG. 10B, M1-like macrophages exhibited larger LDs than M2-like macrophages. Quantification of total LD volume per cell further demonstrated differences in intracellular lipid organization between macrophage states, consistent with distinct metabolic programs, as shown in FIG. 10C. Transmission electron microscopy further demonstrated differences in LD morphology between inflammatory and anti-inflammatory macrophage states, as shown in FIG. 10D. To determine whether these LD characteristics were associated with functional differences, efferocytosis assays were performed. FIG. 10E showed that lipase activity contributed to macrophage efferocytic function and that inhibition of one or more lipases reduced efferocytosis. FIG. 10F further showed that M2-like macrophages had greater efferocytosis activity than M1-like macrophages. When monocyte-derived macrophages from healthy donors and subjects having SLE were compared, basal efferocytosis in monocytes or M1-like macrophages was similar, whereas macrophages driven toward an M2-like state in the presence of C1q exhibited reduced efferocytosis in cells from subjects having SLE relative to healthy donors. These findings indicated that HMGB1 and C1q regulated macrophage lipid metabolism, LD remodeling, and efferocytosis, and further suggested that abnormal LD accumulation or defective LD remodeling in macrophages from subjects having SLE may have contributed to impaired efferocytosis and persistent inflammation. Exposure to HMGB1 increased lipid droplet accumulation and impaired metabolic fitness and efferocytosis, whereas co-treatment with C1q reduced lipid droplet size, increased oxygen consumption rate and the oxygen consumption rate to extracellular acidification rate ratio, increased protein kinase A activity, and restored efferocytosis in a hormone-sensitive lipase-dependent manner, as shown in FIGS. 13A-13D.

### Example 6: Activation of Protein Kinase a and Hormone-sensitive Lipase in Human Cd14+ Monocytes by HMGB1 and C1q

Freshly isolated human CD14+ monocytes were obtained from healthy donors leukopaks, including leukopaks obtained through the New York Blood Bank. Peripheral blood mononuclear cells were isolated by density-gradient centrifugation, and the monocytes were cultured in a serum-free medium. The monocytes were exposed to recombinant disulfide HMGB1, human C1q , or a combination thereof for selected time periods, including about 2 hours, about 4 hours, about 8 hours, about 24 hours, or another suitable time period. To evaluate cyclic adenosine 3′,5′-monophosphate and protein kinase A dependence, the cells were further exposed to dibutyryl-cAMP as a positive control. Phosphodiesterase 3A inhibition was evaluated using milrinone, cilostazol, or both. Hormone-sensitive lipase dependence was evaluated using an HSL inhibitor, including HSL-IN-1.

Protein kinase A activation was quantified using a protein kinase A activity assay and by determining phosphorylated cAMP response element-binding protein relative to total cAMP response element-binding protein. Hormone-sensitive lipase activation was assessed by determining phosphorylated hormone-sensitive lipase at Ser660, phosphorylated hormone-sensitive lipase at Ser563, total hormone-sensitive lipase, or a combination thereof. Lipase activity was quantified using a lipase assay. Culture supernatants were analyzed for one or more cytokines, including TNFα and IL-1β, and for Annexin A1. Integrated miRNA-mRNA network analysis further showed reduced PDE3A expression in macrophages treated with HMGB1 and C1q , together with increased abundance of microRNAs predicted to target PDE3A, including miR-3614-5p, miR-449c-5p, miR-6824-3p, and miR-548av-5p, as shown in FIG. 14. These findings supported a mechanism in which reduced PDE3A expression increased cyclic adenosine 3′,5′-monophosphate-protein kinase A signaling and promoted hormone-sensitive lipase activation.

### Example 7: Evaluation of Lipid Droplet Morphology and Hormone-Sensitive Lipase Localization in Human Monocyte-Derived Macrophages

Human monocyte-derived macrophages (hMDMs) are generated by differentiating CD14+ monocytes in the presence of macrophage colony-stimulating factor. The hMDMs are maintained as M0 macrophages or are polarized to an M1-like state by exposure to HMGB1 or to an M2-like state by exposure to HMGB1 in combination with C1q . Lipid droplet morphology and hormone-sensitive lipase localization are evaluated in the different macrophage states. The cells are cultured on chamber slides, fixed, and stained for perilipin 2, a neutral lipid stain, hormone-sensitive lipase, and a nuclear stain. Images are acquired by confocal microscopy, including z-stack acquisition.

Lipid droplet number, lipid droplet volume per cell, and co-localization of hormone-sensitive lipase with a lipid droplet marker are quantified using image analysis with three-dimensional reconstruction. Live-cell imaging is additionally performed to evaluate transient recruitment of hormone-sensitive lipase to lipid droplets.

### Example 8: Determination of Hormone-Sensitive Lipase Specificity and Downstream Function in HMGB1- and C1q-Treated Human Monocyte-Derived Macrophages

Human monocyte-derived macrophages are generated from CD14+ monocytes obtained from healthy donors, including donor leukopaks obtained through the New York Blood Bank. The CD14+ monocytes are differentiated to M0 macrophages using macrophage colony-stimulating factor. The M0 macrophages are then exposed to HMGB1 to generate M1-like macrophages or to HMGB1 in combination with C1q to generate M2-like macrophages. To determine whether hormone-sensitive lipase is a downstream effector of HMGB1 and C1q signaling and to confirm hormone-sensitive lipase specificity, the macrophages are subjected to LIPE-directed siRNA knockdown, transfection with an HSL-encoding expression construct, or a corresponding control condition. Knockdown or overexpression efficiency is confirmed by quantitative reverse-transcription polymerase chain reaction, immunoblotting, or both.

Control agents that increase adrenergic signaling or cyclic adenosine 3′,5′-monophosphate signaling are included, including isoproterenol, obtained for example from Millipore, dibutyryl-cAMP, obtained for example from Sigma, or both. Isoproterenol binds to a β-adrenergic receptor and stimulates lipolysis by activating hormone-sensitive lipase, including as described in adipocytes, and is therefore used as a control for adrenergic activation of hormone-sensitive lipase. Additional candidate agents described for protein kinase A-dependent activation of hormone-sensitive lipase are evaluated in parallel. Hormone-sensitive lipase activation, protein kinase A activation, cytokine production, and Annexin A1 production or secretion are quantified across the treatment conditions.

Mitochondrial respiration is evaluated using an extracellular flux assay, including a mitochondrial stress test. Mitochondrial membrane potential, mitochondrial morphology, and mitochondrial reactive oxygen species are also quantified. Efferocytosis is measured using fluorescently labeled apoptotic target cells, including apoptotic Jurkat cells, and macrophage internalization is quantified by imaging flow cytometry or microscopy-based analysis. Lipid uptake capacity is further quantified using a fluorescent fatty acid uptake assay.

### Example 9: Evaluation of RAGE- and LAIR-1-Dependent Signaling in HMGB1- and C1q-Mediated Hormone-Sensitive Lipase Activation

The contribution of receptor for advanced glycation end products and leukocyte-associated immunoglobulin-like receptor-1 to HMGB1- and C1q-mediated hormone-sensitive lipase activation is evaluated in human monocyte-derived macrophages. Receptor for advanced glycation end products, leukocyte-associated immunoglobulin-like receptor-1, or both are reduced by siRNA-mediated knockdown prior to exposure of the macrophages to HMGB1, C1q , or both. Protein kinase A activity, phosphorylated hormone-sensitive lipase, lipase activity, lipid droplet remodeling, mitochondrial function, cytokine production, Annexin A1 production or secretion, and efferocytosis are then assessed.

A mimic peptide that reproduces one or more effects of HMGB1 and C1q co-signaling is used as an additional tool to evaluate pathway specificity. Responses induced by the mimic peptide are compared with responses induced by HMGB1 and C1q co-exposure.

### Example 10: In Vivo Evaluation of HMGB1- and C1q-Mediated Hormone-Sensitive Lipase Activation and Efferocytosis in a Sterile Peritonitis Model

Hormone-sensitive lipase activation by HMGB1 and C1q was evaluated in vivo in a sterile peritonitis model. Wild-type mice and C1q-deficient mice were used. The mice received intraperitoneal HMGB1 alone or HMGB1 in combination with C1q . Peritoneal lavage samples were collected after a selected interval, including about 24 hours after administration. Peritoneal myeloid cells were analyzed for phosphorylated hormone-sensitive lipase, protein kinase A pathway activation, gene expression, or a combination thereof.

In vivo efferocytosis was evaluated by administering apoptotic fluorescent target cells into the peritoneal cavity. Apoptotic thymocytes from fluorescent donor mice were used. Peritoneal myeloid cells that have internalized apoptotic target cells awerere then identified by flow cytometry. The results were used to determine whether C1q promoted activation of the protein kinase A-hormone-sensitive lipase axis and enhanced efferocytosis during sterile inflammation. In the sterile peritonitis model, co-administration of HMGB1 and C1q decreased leukotriene B4 levels, increased resolvin D levels, and shifted macrophage-associated gene expression toward a pro-resolving M2-like profile, as shown in FIGS. 15A-15C.

### Example 11: Determination of Hormone-Sensitive Lipase-Dependent Plasma Membrane Remodeling in HMGB1- and C1q-Treated Macrophages

Human monocyte-derived macrophages were generated from CD14+monocytes and were maintained as M0 macrophages or were polarized by exposure to HMGB1 to generate M1-like macrophages or by exposure to HMGB1 in combination with C1q to generate M2-like macrophages. To determine whether hormone-sensitive lipase activity altered plasma membrane fluidity under HMGB1 and HMGB1 plus C1q conditions, membrane fluidity was quantified using an enzyme-linked immunosorbent assay-based membrane fluidity assay. Generalized polarization was also measured using di-4-ANEPPDHQ. The cells were stained with di-4-ANEPPDHQ under live-cell conditions and were examined by live-cell confocal imaging. Generalized polarization values were quantified to compare membrane order and membrane fluidity across treatment conditions.

To determine lipid raft dependence and cholesterol sensitivity, cholesterol levels were modulated using methyl-β-cyclodextrin and matched cholesterol-repletion controls. Raft-associated GM1 domains were quantified by labeling with cholera toxin subunit B. Plasma membrane remodeling was further evaluated by comparing control cells with cells exposed to one or more hormone-sensitive lipase modulators, including isoproterenol, dibutyryl-cAMP, an HSL inhibitor, or combinations thereof.

Untargeted lipidomics was performed to define lipid species in M1-like macrophages, M2-like macrophages, and isoproterenol-treated macrophages, and also in plasma membrane fractions isolated from those cells. Lipids were extracted using an organic solvent extraction method in the presence of internal lipid standards, including SPLASH® LipidoMix internal standards. The extracted lipids were analyzed by ultra-performance liquid chromatography coupled to tandem mass spectrometry. Free fatty acids, diacylglycerols, monoacylglycerols, ceramides, and phospholipid classes were annotated using lipid reference libraries. The resulting dataset was used to identify lipid species associated with hormone-sensitive lipase-dependent plasma membrane remodeling. Macrophages having an M2 or M2-like phenotype exhibited greater plasma membrane fluidity and higher surface expression of CD206 and CD36 than corresponding M1 or M1-like macrophages. Inhibition of hormone-sensitive lipase decreased membrane fluidity and reduced surface expression of one or more scavenger receptors, as shown in FIGS. 16A-16B.

### Example 12: Evaluation of Hormone-Sensitive Lipase-Dependent Scavenger Receptor Organization at the Apoptotic Cell Synapse

Human monocyte-derived macrophages are exposed to HMGB1 or to HMGB1 in combination with C1q and are incubated with apoptotic target cells to form macrophage-apoptotic cell contact sites. To determine whether hormone-sensitive lipase activity promotes synapse-like enrichment and clustering of scavenger receptors at the contact sites, the surface expression and spatial organization of MerTK, CD36, CD206, receptor for advanced glycation end products, and leukocyte-associated immunoglobulin-like receptor-1 are quantified. Receptor distribution is measured by confocal microscopy, and clustering features are quantified, including spot count, polarization rate, and receptor intensity at the apoptotic cell synapse.

To determine whether receptor clustering corresponds to downstream signal transduction, phosphorylation of membrane-proximal receptors is quantified using a phospho-immunoreceptor array that includes leukocyte-associated immunoglobulin-like receptor-1. Blocking antibodies directed to CD36, MerTK, or both are used during efferocytosis to evaluate the contribution of those receptors to synapse formation and receptor signaling. Proximity ligation assays are performed to assess receptor proximity and receptor complex formation at the apoptotic cell synapse. Receptor organization and signaling are compared in control cells and in cells treated with one or more hormone-sensitive lipase activators or inhibitors.

### Example 13: Determination of Hormone-Sensitive Lipase-Dependent Phosphatidylinositol-3,4,5-Trisphosphate Recruitment and Actin Remodeling at the Apoptotic Cell Contact Site

Human monocyte-derived macrophages are transfected with a pleckstrin homology domain-Akt-green fluorescent protein biosensor to quantify phosphatidylinositol-3,4,5-trisphosphate recruitment to apoptotic cell contact sites. CD14+ monocytes are differentiated to M0 macrophages, and the macrophages are transfected with the biosensor using a lipid-based transfection reagent. After transfection, the macrophages are exposed to HMGB1 alone or to HMGB1 in combination with C1q under control conditions or in the presence of one or more hormone-sensitive lipase modulators, including isoproterenol, dibutyryl-cAMP, or an HSL inhibitor. To validate pathway specificity, phosphoinositide 3-kinase inhibitors, including LY294002, wortmannin, or both, are used as additional controls.

Apoptotic target cells are added to the macrophages, and live-cell imaging is performed to track pleckstrin homology domain-Akt-green fluorescent protein recruitment over time. Recruitment kinetics at the macrophage-apoptotic cell interface are quantified using interface masks. Actin cytoskeleton remodeling at the contact site is quantified by phalloidin staining. Activation of Rho family GTPases, including Rho and Rac1, is measured using G-LISA assays. The degree of phosphatidylinositol-3,4,5-trisphosphate recruitment, actin remodeling, and Rho and Rac1 activation is compared across control conditions and hormone-sensitive lipase-modulated conditions to determine whether hormone-sensitive lipase promotes apoptotic cell synapse formation and efferocytosis-related signaling.

### Example 14: Evaluation of Hormone-sensitive Lipase Activation in Macrophages From Subjects With Systemic Lupus Erythematosus

Fresh monocytes and human monocyte-derived macrophages are obtained from healthy donors and from subjects with systemic lupus erythematosus. Blood samples are collected under an institutional review board-approved protocol. Monocytes are isolated using CD16-depleted negative selection of CD14+CD16− monocytes or using a monocyte enrichment method that retains CD14+CD16+ cells. Human monocyte-derived macrophages generated from the isolated monocytes are evaluated in assays including efferocytosis, protein kinase A activation, hormone-sensitive lipase activation, plasma membrane fluidity, and mitochondrial respiration to compare healthy and systemic lupus erythematosus-derived monocytes and macrophages.

After identification of a defect associated with reduced efferocytosis, the cells are treated with isoproterenol and with additional candidate agents selected from compound libraries to identify agents that increase hormone-sensitive lipase activation and efferocytosis. Candidate agents are screened using high-throughput methods, and at least three hormone-sensitive lipase agonists are selected for confirmation by functional readouts including efferocytosis, mitochondrial respiration, plasma membrane remodeling, and receptor organization at apoptotic cell contact sites. Cytokines associated with inflammatory and pro-resolving programs, including TNFα, IL-1β, and Annexin A1, are quantified. Results are analyzed using donor-matched models and, when available, are stratified by clinical metadata.

### Example 15: Identification of Nervonic Acid and Asah1-Associated Lipid Remodeling in Macrophage Polarization.

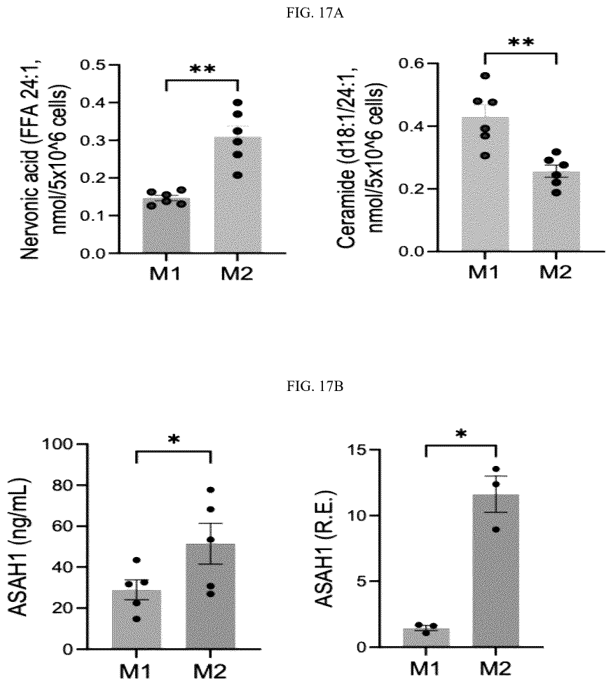

Lipidomics analysis was performed to compare lipid species in inflammatory M1 macrophages and pro-resolving M2 macrophages. As shown in FIG. 17A, nervonic acid (NA; FA 24:1) was increased in M2 macrophages relative to M1 macrophages, whereas ceramide (d18:1/24:1) was reduced in M2 macrophages relative to M1 macrophages. Consistent with the lipidomics findings, acid ceramidase 1 (ASAH1), a ceramide hydrolase that releases sphingosine and free fatty acid, was increased in M2 macrophages at the transcript level and at the protein level, as shown in FIG. 17B. These data indicated that pro-resolving macrophages were associated with increased ASAH1 expression, increased abundance of nervonic acid, and reduced ceramide.

### Example 16: Nervonic Acid Restores Efferocytosis and Oxidative Metabolism in Inflammatory Macrophages

Human and mouse M1 macrophages were treated with nervonic acid at 10 μM for 24 hours, and efferocytosis was then assessed. As shown in FIG. 8A, exogenous nervonic acid significantly enhanced efferocytosis in inflammatory macrophages. At concentrations higher than 10 μM, lipid toxicity was observed. As shown in FIG. 8B, the functional rescue associated with nervonic acid treatment correlated with increased expression of peroxisome proliferator-activated receptor gamma (PPARγ). As shown in FIG. 8C, Seahorse analysis further demonstrated increased basal mitochondrial oxygen consumption rate and increased maximal mitochondrial oxygen consumption rate in nervonic acid-treated M1 macrophages. These data showed that nervonic acid promoted both efferocytic function and oxidative metabolic fitness in inflammatory macrophages.

### Example 17: Nervonic Acid Restores Efferocytosis In Vivo in Lupus-Prone and HMGB1-Treated Mice

Peritoneal macrophages from Sle123 triple congenic lupus-prone mice were evaluated for lipid droplet accumulation and efferocytic function. As shown in FIG. 8D, peritoneal macrophages from SLETC mice exhibited increased lipid droplet content relative to wild-type controls. An HMGB1-induced sterile peritonitis model, as shown in FIGS. 8E-8F, was used to assess in vivo efferocytosis. In this model, HMGB1 suppressed efferocytosis in wild-type mice and in SLETC mice. Administration of a single dose of BSA-conjugated nervonic acid at 10 mg/kg intraperitoneally significantly restored efferocytosis capacity in both strains, as shown in FIG. 8G. These data demonstrated that nervonic acid restored macrophage efferocytic activity in vivo under inflammatory conditions and in a lupus-prone setting.

### Example 18: Evaluation of ASAH1 Dysfunction in Macrophages From Subjects Having Systemic Lupus Erythematosus.

Fresh monocytes and human monocyte-derived macrophages are obtained from healthy donors and from subjects having systemic lupus erythematosus. In some embodiments, the donor population includes female subjects having moderate disease activity and receiving prednisone at 10 mg/day or less. CD14+CD16− monocytes, CD14+CD16+ monocytes, or both are isolated by negative selection and are used immediately or are differentiated into macrophages in macrophage differentiation medium supplemented with recombinant human macrophage colony-stimulating factor for about five days. To evaluate ASAH1-linked lipid remodeling, targeted lipidomics is performed by ultra-performance liquid chromatography-tandem mass spectrometry to quantify ceramide C24:1 species and nervonic acid abundance using internal lipid standards and lipid annotation libraries. ASAH1 expression is quantified by immunoblotting and enzyme-linked immunosorbent assay, and ASAH1 enzymatic activity is measured using an acid ceramidase activity assay. Total lipase activity is quantified by glycerol release from cell lysates, and hormone-sensitive lipase activation is assessed by immunoblotting for phospho-HSL Ser660. In some embodiments, macrophages from subjects having systemic lupus erythematosus exhibit reduced ASAH1 activity, reduced ASAH1 expression, reduced nervonic acid abundance, increased ceramide abundance, reduced hormone-sensitive lipase activation, or a combination thereof relative to macrophages from healthy donors.

### Example 19: Determination of Whether ASAH1 is Required for Nervonic Acid-Associated Efferocytosis

Human monocyte-derived macrophages from healthy donors and selected subjects having systemic lupus erythematosus are transfected with ASAH1 small interfering RNA, an ASAH1 overexpression construct, or an appropriate control. The macrophages are treated with BSA-conjugated nervonic acid at about 10 μM or vehicle for about 24 hours and are then evaluated for efferocytosis by imaging flow cytometry, mitochondrial oxygen consumption rate by Seahorse extracellular flux analysis, and mitochondrial superoxide by MitoSOX using flow cytometry and imaging. Targeted lipidomics is performed to determine whether suppression of ASAH1 reduces nervonic acid abundance and increases ceramide species. To determine whether nervonic acid acts as a metabolic substrate, a membrane remodeling factor, or both, plasma membrane fluidity and scavenger receptor clustering at the efferocytosis synapse are further assessed. In some embodiments, ASAH1 suppression reduces the ability of nervonic acid to enhance efferocytosis, mitochondrial respiration, membrane plasticity, or receptor organization, thereby indicating that ASAH1 contributes to nervonic acid-associated restoration of macrophage function.

### Example 20: Validation of Therapeutic Relevance of Nervonic Acid in Lupus-Prone Mouse Models

Lupus-prone SLETC mice and C57BL/6 control mice are administered HMGB1 intraperitoneally together with BSA-conjugated nervonic acid at about 10 mg/kg intraperitoneally or vehicle, and peritoneal macrophages are analyzed about 24 hours later for efferocytosis. In some embodiments, fluorescently labeled nervonic acid is used to track nervonic acid uptake and localization, and isolated macrophages are further analyzed for lipid droplet features and plasma membrane localization by imaging or mass spectrometry. To assess longer-term therapeutic effects, NZB/W F1 mice are administered nervonic acid or vehicle intraperitoneally every other day for about 2 weeks to about 6 weeks beginning at about 20 weeks of age. Proteinuria, blood urea nitrogen, creatinine, anti-double-stranded DNA antibody titers, renal histopathology, periodic acid-Schiff staining, and immune complex deposition are evaluated. Kidney macrophage lipid droplet accumulation is assessed by LipidTOX staining or perilipin 2 staining together with ASAH1 and PPARγ detection by confocal microscopy. In some embodiments, high-plex spatial proteomic analysis is performed on kidney sections to map ASAH1-positive and perilipin 2-positive macrophages relative to inflammatory niches and tissue injury patterns. In some embodiments, nervonic acid treatment reduces lupus-associated pathology and is associated with increased macrophage efferocytosis, altered lipid droplet remodeling, increased ASAH1-associated pathways, increased PPARγ-associated pathways, or a combination thereof.

### Example 21: Spatial Proteomic and Single-Cell Transcriptomic Analysis of Macrophage Lipid Programs in Human Lupus Nephritis

Human lupus nephritis biopsy specimens and healthy kidney control tissue are analyzed to evaluate macrophage lipid programs and efferocytosis-associated markers in situ. Kidney tissue sections are subjected to high-plex spatial proteomic analysis to detect macrophage markers and lipid-program markers including perilipin 2 (PLIN2), acid ceramidase 1 (ASAH1), peroxisome proliferator-activated receptor gamma (PPARγ), MER proto-oncogene, tyrosine kinase (MerTK), cluster of differentiation 36 (CD36), and complement component C1q . In parallel, one or more available single-cell RNA sequencing datasets from subjects having lupus nephritis are analyzed to identify macrophage subsets enriched for ASAH1, PPARγ, and C1q expression.

The transcriptomic analysis identifies a damage-associated macrophage population characterized by expression of ASAH1, PPARγ, and C1q . Spatial proteomic findings are integrated with transcriptomic clustering to determine whether such macrophages exhibit molecular features associated with lipid droplet remodeling, efferocytosis, inflammatory activation, and pro-resolving activity. Macrophage subsets expressing ASAH1, PPARγ, and C1q are localized within regions of glomerular injury, tubular injury, inflammatory infiltrates, immune complex deposition, and fibrotic regions, and are further evaluated for co-expression of PLIN2, MerTK, and CD36. Comparison with healthy kidney control tissue identifies disease-associated changes in macrophage abundance, localization, and lipid-program marker expression. These analyses are used to evaluate translational relevance of the disclosed lipid remodeling pathways and to identify macrophage populations associated with lupus nephritis severity, tissue damage, and therapeutic responsiveness.

### Example 22: Evaluation of Alternative Mechanisms and Combination Conditions For Restoration of Macrophage Efferocytosis

Macrophages exhibiting impaired efferocytosis are treated with nervonic acid and are evaluated to determine whether restoration of function is associated predominantly with improved mitochondrial fitness, improved membrane fluidity, or both. Mitochondrial fitness is assessed by mitochondrial oxygen consumption rate, oxidative metabolism, and mitochondrial reactive oxygen species measurements. Membrane-associated restoration is assessed by membrane fluidity analysis, lipid trafficking analysis, and evaluation of phagocytic synapse assembly and receptor clustering. Where nervonic acid alone does not fully restore efferocytosis, macrophages are further treated under combination conditions including nervonic acid together with a peroxisome proliferator-activated receptor gamma agonist, a protein kinase A activator, a hormone-sensitive lipase activator, or a combination thereof. Such combination conditions are evaluated for their ability to restore membrane phosphoinositide competence, improve phagocytic synapse assembly, and increase efferocytosis.

### Example 23: Optimization of Nervonic Acid Delivery and Evaluation of Additional Anti-Inflammatory Lipid Species

Animal models exhibiting inflammatory or lupus-associated macrophage dysfunction are treated with nervonic acid under varying dose levels, dosing schedules, formulations, or routes of administration to improve tissue exposure and macrophage response. Where systemic administration provides insufficient renal bioavailability or undesired off-target distribution, nervonic acid is formulated in lipid nanoparticles or other delivery vehicles and kidney macrophage endpoints are assessed to optimize delivery. In parallel, one or more additional anti-inflammatory lipid species identified by lipidomic analysis, including phosphatidylinositol 18:0_18:0 or lysophosphatidylinositol 18:0, are tested alone or in combination with nervonic acid. The tested conditions are evaluated for effects on macrophage efferocytosis, lipid droplet remodeling, mitochondrial function, membrane properties, inflammatory resolution, renal pathology, or combinations thereof.

### Example 24: Materials and Methods

### Materials

Orlistat (10-100 uM), T-863 (50 μM), Lalistat 1 (5 uM), Atglistatin (20 uM) and MJN110 (5 uM) were purchased from Cayman Chemical (Ann Arbor, MI), HSL-IN-1 (5-15 uM) from MedChemExpress (Monmouth Junction, NJ), Dibutyryl-cAMP (50 uM) from Stem Cell Technology (Vancouver, Canada).

### Monocyte Isolation and Cell Culture

Human peripheral blood mononuclear cells (PBMCs) were isolated from whole human blood using density gradient centrifugation with Ficoll-Paque Plus (Cytiva, Marlborough, MA). Leukopaks were obtained from healthy donors recruited through the New York Blood Center (New York, NY). Monocytes were enriched from PBMCs through negative selection, utilizing human monocyte enrichment kits (Stem Cell Technology). The purity of the monocytes (≥90%) was assessed using a flow cytometer (LSRFortessa; BD, Franklin Lakes, NJ). The purified monocytes (1.5×10\hat{ } cells per mL) were cultured with 20-40 ng/mL of M-CSF (PeproTech, Cranbury, NJ) for 4-5 days in a 5% CO2 incubator at 37 °C to differentiate into macrophages (M0). The M0 macrophages were then stimulated in 24-well plates (1×10\hat{ } cells per well) with either IFN-γ (20 ng/mL, PeproTech) and LPS (100 ng/mL, Merck KGaA, Darmstadt, Germany) to generate M1-like macrophages (M1) or with IL-4 (20 ng/mL, PeproTech) to generate M2-like macrophages (M2), with or without inhibitors, for 24 hours.

### Preparation of Bone Marrow-Derived Macrophages (BMDMs)

Bone marrow cells were extracted from the femurs and tibias of C57BL/6 mice purchased from Jackson Laboratory (Bar Harbor, ME). The animals were maintained in a conventional animal housing facility and euthanized according to approved protocols by the Feinstein Institutes IACUC committee (2022-022). The marrow was flushed with RPMI 1640 medium supplemented with 10% fetal bovine serum (FBS), 1% penicillin/streptomycin, and 20 ng/mL murine M-CSF (PeproTech) to promote differentiation into macrophages. Cells were cultured in a humidified incubator at 37 °C with 5% CO2. After three days, non-adherent cells were removed, and fresh medium was added with M-CSF. On day five, M0 cells were differentiated into M1 (murine IFN-γ, 20 ng/mL, PeproTech) and LPS (100 ng/mL, Merck) or M2 (murine IL-4, 20 ng/mL, PeproTech) macrophages. Cells were harvested for experiments after 24 hours.

### qRT-PCR

Total RNA was extracted from cells using Direct-zol RNA Miniprep Kits or Direct-zol-96 RNA Kits (Zymo Research, Irvine, CA). Complementary DNA (cDNA) was synthesized with the iScript cDNA synthesis kit (Bio-Rad, Hercules, CA). Real-time PCR reactions were conducted on a LightCycler 480 II (Roche, Basel, Switzerland) using LightCycler 480 Probes Master (Roche). Primers for IL1B (Hs01555410_m1), HIF1A (Hs00153153_m1), MERTK (Hs1031973_m1), MRC1/CD206 (Hs00267207_m1), PPARG (Hs01115513_m1), PNPLA2/ATGL (Hs00982042_m1), LIPE/HSL (Hs00943410_m1), and HPRT1 (Hs99999909_m1) were obtained from Thermo Fisher Scientific (Waltham, MA). The genes of interest were normalized to the expression of housekeeping genes, and relative expression (R.E.) was calculated using the 2\hat{ }−ΔCt method.

### Flow Cytometry

Cells were suspended in HBSS with 3% BSA and incubated with Fc Receptor Blocking Solution (BioLegend, San Diego, CA) for 5 minutes at 4 °C. The cells were then incubated for 30 minutes at 4 °C with a panel of antibodies: Pacific Blue-anti-human CD14 (BD), phycoerythrin (PE)-Cy7-anti-CD36 (BioLegend), PE-Cy7-anti-CD274 (BD), and Brilliant Violet 711-anti-CD163 (BioLegend). Fixable Viability Dye (FVD) eFluor780 (Thermo Fisher Scientific) was included as well. After incubation, the cells were washed and fixed in 4% paraformaldehyde (PFA, Sigma) and stored in the dark at 4 °C until analysis. Sample acquisition was performed using either the LSR Fortessa or Symphony flow cytometers (BD), and data were analyzed using FlowJo software (BD).

### Efferocytosis Assay

Efferocytosis was quantified using the Efferocytosis Assay Kit (Cayman Chemical), following the manufacturer's guidelines. Initially, M0 macrophages were stained with CytoTell™ Blue and polarized as previously described. Jurkat cells, clone E6-1 (ATCC, Manassas, VA), were labeled with CFSE and treated with Staurosporine overnight to induce apoptosis. These apoptotic Jurkat cells were washed twice and then co-cultured with the stained macrophages at a 1:2 ratio (effector cells: Jurkat cells) for 2 hours in a humidified incubator at 37 °C with 5% CO2. After incubation, cells were washed and fixed in 4% buffered formaldehyde (Sigma) for 20 minutes. The samples were then analyzed using the Amnis ImageStreamX Mk II Imaging Flow Cytometer (Cytek Biosciences, Fremont, CA) and the LSR Fortessa (BD). Data were processed using IDEAS software (Cytek Biosciences) or FlowJo software (BD) to evaluate CFSE positivity in the macrophages.

In a parallel assay using mouse BMDMs, apoptotic thymocytes from C57BL/6 mice, treated overnight with Staurosporine, were co-cultured with CFSE-stained macrophages at 1:5 for 90 minutes. Following this, harvested cells were stained with Pacific Blue™-anti-mouse F4/80 antibody, washed, and fixed the same as in the procedure described above.

### Neutral Lipid Staining

HCS LipidTOX™ Green Neutral Lipid Stain was used to evaluate the intensity of neutral lipids according to the manufacturer's instructions. Briefly, harvested macrophages were fixed in 4% PFA and then stained with HCS LipidTOX™ Green Neutral Lipid Stain. After washing, cells were analyzed using the Amnis ImageStreamX Mk II Imaging Flow Cytometer (Cytek Biosciences). The geometric mean fluorescence intensity among single cells was calculated using IDEAS software (Cytek Biosciences).

### Confocal Microscopy Assay Immunofluorescence Microscopy

M0-macrophages were stimulated in 8-well chambers (2×10\hat{ } cells per well) for 24 hours. The cells were washed, fixed with 4% PFA, and permeabilized using BD Perm/Wash™ Buffer (BD). Immunofluorescence staining was carried out with primary antibodies against ADRP/Perilipin-2 (Thermo Fisher Scientific) or HSL (Cell Signaling Technology, Danvers, MA), followed by Alexa Fluor® 594 or 488-conjugated anti-rabbit IgG antibodies. After washing, LipidTOX™ Deep Red Neutral Lipid Stain and DAPI were used for staining neutral lipids and nuclei, respectively. Imaging was performed using a confocal microscope (60× with oil, LSM900, Zeiss, Oberkochen, Germany), and the images were analyzed using Imaris software (Oxford Instruments). The number and size of LD per cell were quantified based on ADRP/Perilipin-2 staining, and colocalization analysis was conducted using the fluorescence thresholding method.

### Lipase and Protein Kinase a (PKA) Activity Assays

Lipase activity was measured using the Lipase Assay Kit (Abcam, Cambridge, UK) according to the manufacturer's instructions. In this assay, macrophage lysate hydrolyzes a triglyceride substrate in the presence of calcium, forming glycerol. The glycerol was then quantified enzymatically by monitoring the absorbance change of a probe at 570 nm (OD=570 nm). PKA activity was assessed using the PKA Colorimetric Activity Kit (Thermo Fisher Scientific) according to the manufacturer's instructions.

### Western Blot

Total protein extracts from macrophages (1×10\hat{ }6 cells) were prepared using RIPA lysis buffer containing protease and phosphatase inhibitors (Thermo Fisher Scientific). The samples were combined with Laemmli sodium dodecyl sulfate (SDS) buffer, boiled, and subjected to immunoblot analysis utilizing 4-10% gradient SDS-PAGE. The proteins were then transferred to a PVDF membrane (Thermo Fisher Scientific). For detection, a primary antibody against phospho-HSL (Ser660) (Cell Signaling Technology) was used. ECL Plus Substrate (Thermo Fisher Scientific) was employed to visualize secondary antibodies conjugated with HRP. The membrane was scanned using the Sapphire Biomolecular Imager (Azure Biosystems, Dublin, CA).

### Plasmids and Transient Transfection

M0 macrophages in 24-well plates were transfected using Lipofectamine LTX (Thermo Fisher Scientific) according to the manufacturer's protocol. The cells were transfected with either a PNPLA2-encoding plasmid (ATGL, GeneCopoeia, Rockville, MD), a LIPE-encoding plasmid (HSL, GeneCopoeia), or a control plasmid (GeneCopoeia). All plasmids carry mCherry tags. qRT-PCR confirmed the efficiency of ATGL and HSL overexpression.

For the efferocytosis assay, transfected macrophages were co-cultured with apoptotic Jurkat cells without prior staining with CytoTell™. After co-culture, the cells were stained with APC-Cy7-CD14 (BioLegend) prior to fixation and analyzed using the LSRFortessa (BD). Data were evaluated using FlowJo software (BD), assessing CFSE positivity in mCherry and CD14-positive macrophages.

### ELISA

The levels of IL-1β and TNFα in the culture supernatants from human and mouse sources were quantified using the IL-1 beta/IL-1F2 DuoSet ELISA and TNF-alpha DuoSet ELISA kits (R&D Systems, Minneapolis, MN). All assays were performed strictly according to the manufacturer's instructions.

### Transmission Electron Microscopy

M0 macrophages were stimulated in 6-well plates (5×10\hat{ }5 cells per well) as described above and then fixed for 30 minutes with 2.5% glutaraldehyde in 0.1 M sodium cacodylate buffer (Thermo Fisher Scientific). The cells were subsequently post-fixed with 1% osmium tetroxide, followed by 2% uranyl acetate. They were then dehydrated through a graded series of ethanol, lifted from the monolayer using propylene oxide, and embedded as a loose pellet in LX112 resin (LADD Research Industries, Burlington, VT) in Eppendorf tubes. Ultrathin sections were cut on a Leica Ultracut UC7 (Wetzlar, Germany), stained with uranyl acetate followed by lead citrate, and examined using a JEOL 1400Plus transmission electron microscope (Tokyo, Japan) at 120 kV (1500×or 5000×) at the Analytical Imaging Facility at the Albert Einstein College of Medicine. The size and density of LD (5000×) were analyzed using ImageJ (Schneider, Rasband and Eliceiri, 2012).

### Statistical Analysis

Statistical analysis was conducted using GraphPad Prism 10 (GraphPad Inc., La Jolla, California), R, or both. In studies comparing two macrophage groups, a paired Student's t-test was used. In studies including more than two groups, one-way analysis of variance or two-way analysis of variance was used, as appropriate, followed by post hoc pairwise comparisons adjusted for multiple comparisons using the Tukey method. The statistical analyses used for particular datasets were indicated in the corresponding figure legends. Variables were assessed for distributional properties before selection of the statistical test. All tests were two-tailed, and a p-value of less than 0.05 was considered statistically meaningful.

Donor selection is structured to account for disease prevalence and biological variability. Where systemic lupus erythematosus donor samples are used, the donor population is enriched for female subjects. Healthy donor and disease donor cohorts are selected to account for heterogeneity in responses to high mobility group box 1 and complement component C1q . In representative studies, about 6 to about 10 healthy donors are used per experiment, about 24 systemic lupus erythematosus donors are included, and about 20 matched healthy donors are included for comparative studies. Murine studies use about 10 animals per group.

Power calculations are performed using preliminary data and standard power analysis software. In representative paired comparisons, an effect size of about 1.5, an alpha level of 0.05, and a statistical power of 0.8 are used to determine the minimum donor number. Independent biological replicates and technical replicates support the stated conclusions.

Image-based experiments are designed, acquired, and quantified using blinded methods. Lipidomic analyses include internal standards and quality-control samples. In vivo studies incorporate randomization and blinded outcome assessment. Standardized quality-control procedures are used across studies to improve rigor and reproducibility.

### Specific Embodiments

Non-limiting specific embodiments are described below each of which is considered to be within the present disclosure.

Embodiment 1. A composition, including a lipase modulator (e.g., activator, inhibitor).

Embodiment 2. The composition of Embodiment 1, where the lipase modulator modulates an intracellular lipase.

Embodiment 3. The composition of Embodiment 2, where the intracellular lipase is selected from the group consisting of adipose triglyceride lipase (ATGL), hormone-sensitive lipase (HSL), monoglyceride lipase (MGL), lysosomal acid lipase (LAL), N-acylsphingosine amidohydrolase 1 (ASAH1), and carboxyl ester lipase (CEL).

Embodiment 4. The composition of any one of Embodiments 1-3, further including a pharmaceutically acceptable excipient.

Embodiment 5. The composition of any one of Embodiments 1-4, where the lipase modulator is a lipase activator.

Embodiment 6. The composition of any one of Embodiments 1-4, where the lipase modulator is a lipase inhibitor.

Embodiment 7. The composition of Embodiment 6, where the lipase inhibitor is selected from the group consisting of Atglistatin, HSL-IN-1, MJN110, Orlistat, and combinations thereof.

Embodiment 8. The composition of Embodiment 5, where the lipase activator increases HSL activity, increases ATGL activity, or increases both HSL activity and ATGL activity.

Embodiment 9. The composition of any one of Embodiments 1-8, where the composition is for treating dysregulation of lipid metabolism, impaired macrophage efferocytosis, impaired macrophage function, or a combination thereof.

Embodiment 10. A method, including administering a therapeutically effective amount of the composition of any one of Embodiments 1-8 to a subject suffering from dysregulation of lipid metabolism, impaired macrophage efferocytosis, impaired macrophage function, or a combination thereof, thereby treating the subject.

Embodiment 11. The method of Embodiment 10, where the therapeutically effective amount regulates efferocytosis, macrophage function, lipid metabolism, or a combination thereof.

Embodiment 12. The method of any one of Embodiments 10-11, where a disease associated with dysregulation of lipid metabolism, impaired macrophage efferocytosis, impaired macrophage function, or a combination thereof is selected from the group consisting of an autoimmune disease, a neurodegenerative disease, diabetes, cancer, an inflammatory disease, chronic obstructive pulmonary disease (COPD), atherosclerosis, multiple sclerosis (MS), and combinations thereof.

Embodiment 13. The method of Embodiment 12, where the autoimmune disease is systemic lupus erythematosus (SLE) or lupus nephritis.

Embodiment 14. The method of any one of Embodiments 10-13, where administering increases phosphorylation of HSL at Ser660 in macrophages of the subject.

Embodiment 15. The method of any one of Embodiments 10-14, where administering increases translocation of HSL to lipid droplets in macrophages of the subject.

Embodiment 16. Use of a lipase modulator composition of any one of Embodiments 1-9 in the manufacture of a medicament for the treatment of a disease associated with dysregulation of lipid metabolism, impaired macrophage efferocytosis, impaired macrophage function, or a combination thereof.

Embodiment 17. A method of treating dysregulation of lipid metabolism, impaired macrophage efferocytosis, impaired macrophage function, or a combination thereof in a subject, including administering to the subject an agent that increases a cyclic adenosine monophosphate (cAMP)-protein kinase A (PKA)-HSL pathway in macrophages of the subject, thereby increasing macrophage efferocytosis in the subject.

Embodiment 18. The method of Embodiment 17, where the agent decreases phosphodiesterase 3A (PDE3A) activity or expression.

Embodiment 19. The method of Embodiment 18, where the agent includes a phosphodiesterase 3 inhibitor.

Embodiment 20. The method of Embodiment 19, where the phosphodiesterase 3 inhibitor includes milrinone or cilostazol.

Embodiment 21. The method of Embodiment 18, where the agent includes a microRNA that decreases PDE3A expression.

Embodiment 22. The method of Embodiment 21, where the microRNA includes miR-449c-5p, miR-6824-3p, miR-548av-5p, or a combination thereof.

Embodiment 23. The method of Embodiment 17, where the agent includes a beta-adrenergic receptor agonist or vagus nerve stimulation.

Embodiment 24. The method of Embodiment 23, where the beta-adrenergic receptor agonist includes a beta2-adrenergic receptor agonist, isoproterenol, norepinephrine, or a combination thereof.

Embodiment 25. A method of increasing efferocytosis in a macrophage, including contacting the macrophage with C1q in the presence of high mobility group box 1 (HMGB1), thereby increasing PKA activity, HSL activity, and efferocytosis in the macrophage.

Embodiment 26. The method of Embodiment 25, where contacting the macrophage modulates signaling through receptor for advanced glycation end products (RAGE), leukocyte-associated immunoglobulin-like receptor-1 (LAIR-1), or both.

Embodiment 27. A method of treating impaired macrophage efferocytosis in a subject, including administering to the subject C1q or an agent that increases C1q activity, thereby increasing a pro-resolving macrophage phenotype characterized by increased PKA activity, increased HSL activity, and increased efferocytosis in the subject.

Embodiment 28. The method of Embodiment 27, where the subject has SLE or lupus nephritis.

Embodiment 29. A pharmaceutical composition, including nervonic acid or a pharmaceutically acceptable salt thereof and a pharmaceutically acceptable excipient.

Embodiment 30. The pharmaceutical composition of Embodiment 29, where the nervonic acid is conjugated to serum albumin.

Embodiment 31. The pharmaceutical composition of Embodiment 29, where the pharmaceutical composition further includes serum albumin.

Embodiment 32. The pharmaceutical composition of any one of Embodiments 29-31, where the pharmaceutical composition is formulated for intraperitoneal administration.

Embodiment 33. The composition of any one of Embodiments 1-9, further including nervonic acid or a pharmaceutically acceptable salt thereof.

Embodiment 34. The composition of Embodiment 33, where the nervonic acid is conjugated to serum albumin.

Embodiment 35. A method of treating impaired macrophage efferocytosis in a subject, including administering to the subject nervonic acid or a pharmaceutically acceptable salt thereof, thereby increasing macrophage efferocytosis in the subject.

Embodiment 36. The method of Embodiment 35, where administering nervonic acid increases mitochondrial respiration in macrophages of the subject.

Embodiment 37. The method of Embodiment 35, where administering nervonic acid increases peroxisome proliferator-activated receptor gamma (PPARγ) expression in macrophages of the subject.

Embodiment 38. The method of any one of Embodiments 35-37, where administering nervonic acid includes intraperitoneal administration.

Embodiment 39. The method of Embodiment 38, where nervonic acid is administered at about 10 mg/kg.

Embodiment 40. The method of any one of Embodiments 35-39, further including administering to the subject a second agent selected from the group consisting of a PPARγ agonist, a PKA activator, a HSL activator, an ATGL activator, and combinations thereof.

Embodiment 41. A method of treating impaired macrophage efferocytosis in a subject, including administering to the subject an agent that increases activity of ASAH1 in a macrophage, thereby increasing macrophage efferocytosis in the subject.

Embodiment 42. The method of Embodiment 41, where increasing activity of ASAH 1 increases nervonic acid abundance in the macrophage.

Embodiment 43. A method of increasing macrophage efferocytosis, including increasing expression of LIPE in a macrophage, thereby increasing HSL activity and macrophage efferocytosis.

Embodiment 44. The method of Embodiment 43, where increasing expression of LIPE includes introducing into the macrophage a nucleic acid encoding LIPE.

Embodiment 45. A method of increasing macrophage efferocytosis, including increasing activity of ATGL in a macrophage, thereby increasing macrophage efferocytosis.

Embodiment 46. The method of Embodiment 45, where increasing activity of ATGL includes increasing expression of PNPLA2 in the macrophage.

Embodiment 47. An in vitro method of identifying a pro-resolving macrophage population in a biological sample, including detecting an increased level of nervonic acid relative to a reference level, thereby identifying the pro-resolving macrophage population in the biological sample.

Embodiment 48. The in vitro method of Embodiment 47, where detecting includes mass spectrometry-based lipidomics.

Embodiment 49. The in vitro method of Embodiment 47 or 48, where detecting further includes detecting an increased level of a phosphatidylinositol species relative to the reference level.

Embodiment 50. The in vitro method of Embodiment 49, where the phosphatidylinositol species includes PI(18:0_18:0).

Embodiment 51. The in vitro method of any one of Embodiments 47-50, where the biological sample includes peripheral blood mononuclear cells, isolated monocytes, macrophages, tissue, lavage fluid, biopsy material, or a combination thereof.

Embodiment 52. The in vitro method of any one of Embodiments 47-51, further including selecting a subject for treatment with nervonic acid, a phosphodiesterase 3 inhibitor, a beta-adrenergic receptor agonist, C1q , or a combination thereof based on identification of the pro-resolving macrophage population.

Embodiment 53. The in vitro method of any one of Embodiments 47-52, where the reference level is obtained from a healthy control, an inflammatory macrophage population, an untreated sample, a baseline sample from the same subject, or a combination thereof.

Embodiment 54. An in vitro method of identifying a candidate agent for ability to promote pro-resolving macrophage activity, including contacting a macrophage with the candidate agent and selecting the candidate agent when the macrophage exhibits increased efferocytosis relative to a control macrophage not contacted with the candidate agent.

Embodiment 55. The in vitro method of Embodiment 54, where selecting includes determining increased phosphorylation of HSL at Ser660 relative to the control macrophage.

Embodiment 56. The in vitro method of Embodiment 54 or 55, where selecting further includes determining increased membrane fluidity in the macrophage.

Embodiment 57. The in vitro method of any one of Embodiments 54-56, where selecting further includes determining increased receptor clustering, receptor polarization, synapse formation, phagocytic cup formation, actin remodeling, phosphoinositide-dependent signaling, or a combination thereof at an apoptotic cell contact site.

Embodiment 58. The in vitro method of Embodiment 57, where the receptor clustering or receptor polarization includes organization of one or more efferocytosis-associated receptors selected from the group consisting of MERTK, TIM-4, CD36, CD206, RAGE, LAIR-1, and combinations thereof.

Embodiment 59. The in vitro method of any one of Embodiments 54-58, where the macrophage is contacted with the candidate agent in the presence of HMGB1.

Embodiment 60. The in vitro method of any one of Embodiments 54-59, where the candidate agent is selected from the group consisting of a phosphodiesterase 3 inhibitor, a beta-adrenergic receptor agonist, C1q , nervonic acid, and combinations thereof.

Embodiment 61. An in vitro method of evaluating kidney tissue, including spatially detecting expression of ASAH1, PLIN2, PPARγ, MERTK, CD36, or a combination thereof in the kidney tissue, thereby identifying a macrophage population associated with lipid droplet remodeling, efferocytosis, or both.

Embodiment 62. The in vitro method of Embodiment 61, where the kidney tissue is obtained from a subject having lupus nephritis.

Embodiment 63. The in vitro method of Embodiment 61 or 62, where spatially detecting includes immunohistochemistry, immunofluorescence, spatial proteomics, spatial transcriptomics, imaging mass spectrometry, or a combination thereof.

Embodiment 64. The in vitro method of any one of Embodiments 61-63, where spatially detecting further includes detecting CD68, CD14, CD206, or a combination thereof.

Embodiment 65. The in vitro method of any one of Embodiments 61-64, where a spatially detected marker pattern is used to assess disease state, monitor treatment response, identify a target tissue macrophage population, or evaluate an effect of treatment with a lipase modulator, nervonic acid, C1q , a phosphodiesterase 3 inhibitor, a beta-adrenergic receptor agonist, or a combination thereof.

Embodiment 66. An in vitro method of reducing amyloid-beta-induced lipid droplet accumulation in a myeloid cell, including contacting the myeloid cell with C1q , thereby reducing the amyloid-beta-induced lipid droplet accumulation in the myeloid cell.

Embodiment 67. The in vitro method of Embodiment 66, where the myeloid cell is a microglia-like cell.

Embodiment 68. A method of treating a neuroinflammatory disease, a neurodegenerative disease, or both in a subject having amyloid-beta-associated myeloid-cell dysfunction, including administering to the subject C1q , a lipase modulator, a phosphodiesterase 3 inhibitor, an adrenergic agonist, nervonic acid, or any combination thereof.

Embodiment 69. The method of Embodiment 68, where the neurodegenerative disease includes Alzheimer's disease.

Embodiment 70. A method of treating a cancer in a subject, including administering to the subject a therapeutically effective amount of a lipase inhibitor and thereby inhibiting lipid metabolism in a tumor-associated macrophage.

Embodiment 71. The method of Embodiment 70, where the lipase inhibitor is selected from the group consisting of Orlistat, Atglistatin, HSL-IN-1, MJN110, and any combination thereof.

Embodiment 72. The method of Embodiment 70 or 71, further including administering to the subject chemotherapy, immunotherapy, radiation therapy, or any combination thereof.

Embodiment 73. A pharmaceutical composition, including nervonic acid conjugated to serum albumin and a pharmaceutically acceptable excipient.

Embodiment 74. The pharmaceutical composition of Embodiment 73, where the serum albumin is bovine serum albumin.

Embodiment 75. The pharmaceutical composition of Embodiment 73, where the serum albumin is human serum albumin.

Embodiment 76. The pharmaceutical composition of Embodiment 73, where the pharmaceutical composition is formulated for intraperitoneal administration.

Embodiment 77. The pharmaceutical composition of Embodiment 73, where the pharmaceutical composition is formulated as an injectable solution.

Embodiment 78. The pharmaceutical composition of Embodiment 73, where the nervonic acid is conjugated to the serum albumin at a 3:1 molar ratio.

Embodiment 79. The pharmaceutical composition of Embodiment 73, further including serum albumin.

Embodiment 80. A method of treating impaired macrophage efferocytosis in a subject having systemic lupus erythematosus or lupus nephritis, including administering to the subject a therapeutically effective amount of an agent selected from the group consisting of C1q, milrinone, cilostazol, isoproterenol, norepinephrine, and any combination thereof, thereby increasing macrophage efferocytosis in the subject.

Embodiment 81. The method of Embodiment 80, where the subject has lupus nephritis.

Embodiment 82. The method of Embodiment 80, where the agent includes C1q.

Embodiment 83. The method of Embodiment 80, where the agent includes milrinone or cilostazol.

Embodiment 84. The method of Embodiment 80, where the agent includes isoproterenol or norepinephrine.

Embodiment 85. The method of Embodiment 80, where administering the agent increases phosphorylation of hormone-sensitive lipase at Ser660 in macrophages of the subject.

Embodiment 86. The method of Embodiment 80, where administering the agent increases mitochondrial respiration in macrophages of the subject.

Embodiment 87. An in vitro method of identifying a candidate agent that increases macrophage efferocytosis, including: contacting a macrophage with a candidate agent, contacting the macrophage with a labeled apoptotic cell, measuring engulfment of the labeled apoptotic cell by the macrophage relative to a control macrophage not contacted with the candidate agent, and selecting the candidate agent when the macrophage contacted with the candidate agent exhibits increased engulfment relative to the control macrophage.

Embodiment 88. The in vitro method of Embodiment 87, where the macrophage is a human monocyte-derived macrophage differentiated with macrophage colony-stimulating factor.

Embodiment 89. The in vitro method of Embodiment 87, where the labeled apoptotic cell is a CFSE-labeled apoptotic Jurkat cell.

Embodiment 90. The in vitro method of Embodiment 87, where the macrophage is contacted with the candidate agent in the presence of high mobility group box 1.

Embodiment 91. The in vitro method of Embodiment 87, where selecting the candidate agent further includes determining increased phosphorylation of hormone-sensitive lipase at Ser660 in the macrophage relative to the control macrophage.

Embodiment 92. The in vitro method of Embodiment 87, where the candidate agent is selected from the group consisting of C1q, milrinone, cilostazol, isoproterenol, norepinephrine, nervonic acid, and any combination thereof.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A pharmaceutical composition, comprising:
a therapeutic agent selected from the group consisting of a lipase modulator, nervonic acid or a pharmaceutically acceptable salt thereof, C1q , a phosphodiesterase 3 inhibitor, a beta-adrenergic receptor agonist, and any combination thereof; and
a pharmaceutically acceptable excipient.

2. The pharmaceutical composition of claim 1, wherein the lipase modulator modulates an intracellular lipase selected from the group consisting of adipose triglyceride lipase, hormone-sensitive lipase, monoglyceride lipase, lysosomal acid lipase, N-acylsphingosine amidohydrolase 1, and carboxyl ester lipase.

3. The pharmaceutical composition of claim 1, wherein the therapeutic agent comprises nervonic acid or the pharmaceutically acceptable salt thereof.

4. The pharmaceutical composition of claim 3, wherein the nervonic acid is conjugated to serum albumin.

5. The pharmaceutical composition of claim 1, wherein the therapeutic agent comprises a phosphodiesterase 3 inhibitor.

6. The pharmaceutical composition of claim 5, wherein the phosphodiesterase 3 inhibitor comprises milrinone or cilostazol.

7. The pharmaceutical composition of claim 1, wherein the therapeutic agent comprises C1q.

8. A method of treating dysregulation of lipid metabolism, impaired macrophage efferocytosis, impaired macrophage function, or a disease associated therewith in a subject, comprising:
administering to the subject a therapeutically effective amount of a therapeutic agent selected from the group consisting of a lipase modulator, nervonic acid or a pharmaceutically acceptable salt thereof, C1q , a phosphodiesterase 3 inhibitor, a beta-adrenergic receptor agonist, and combinations thereof; and
thereby increasing macrophage efferocytosis in the subject.

9. The method of claim 8, wherein the disease associated therewith is an autoimmune disease.

10. The method of claim 9, wherein the autoimmune disease is systemic lupus erythematosus or lupus nephritis.

11. The method of claim 8, wherein the therapeutic agent comprises nervonic acid or the pharmaceutically acceptable salt thereof.

12. The method of claim 8, wherein the therapeutic agent comprises a phosphodiesterase 3 inhibitor.

13. The method of claim 8, wherein the therapeutic agent comprises a beta-adrenergic receptor agonist.

14. The method of claim 8, wherein administering the therapeutic agent increases phosphorylation of hormone-sensitive lipase at Ser660 in macrophages of the subject.

15. An in vitro method of identifying a candidate agent that promotes pro-resolving macrophage activity, comprising:
contacting a macrophage with a candidate agent;
determining whether the macrophage exhibits increased efferocytosis relative to a control macrophage not contacted with the candidate agent; and
selecting the candidate agent when the macrophage exhibits increased efferocytosis relative to the control macrophage.

16. The in vitro method of claim 15, wherein selecting the candidate agent further comprises determining increased phosphorylation of hormone-sensitive lipase at Ser660 in the macrophage relative to the control macrophage.

17. The in vitro method of claim 15, wherein selecting the candidate agent further comprises determining increased membrane fluidity in the macrophage relative to the control macrophage.

18. The in vitro method of claim 15, wherein selecting the candidate agent further comprises determining increased receptor clustering or increased receptor polarization of one or more receptors selected from the group consisting of MERTK, TIM-4, CD36, CD206, receptor for advanced glycation end products, leukocyte-associated immunoglobulin-like receptor-1, and combinations thereof at an apoptotic cell contact site.

19. The in vitro method of claim 15, wherein the macrophage is contacted with the candidate agent in the presence of high mobility group box 1.

20. The in vitro method of claim 15, wherein the candidate agent is selected from the group consisting of a phosphodiesterase 3 inhibitor, a beta-adrenergic receptor agonist, C1q , nervonic acid, a lipase modulator, and combinations thereof.
